(19) United States
(12) Patent Application Publication
(10) Pub. No.: US 2017/0268071 A1
(43) Pub. Date: Sep. 21, 2017

(54) COMPOSITIONS AND METHODS OF USING TRANSFER RNAS (tRNAs)

(71) Applicant: THOMAS JEFFERSON UNIVERSITY, Philadelphia, PA (US)

(72) Inventor: Isidore RIGOUTSOS, New York, NY (US)

(73) Assignee: Thomas Jefferson University

(21) Appl. No.: 15/521,828

(22) PCT Filed: Oct. 27, 2015
(86) PCT No.: PCT/US2015/057643

§ 371 Date: Apr. 25, 2017

Related U.S. Application Data
(60) Provisional application No. 62/122,711, filed on Oct. 28, 2014.

(51) Int. Cl. C12Q 1/68

(52) U.S. Cl. CPC C12Q 1/6886; C12Q 2600/106; C12Q 2600/178

(57) ABSTRACT

The present invention includes a method for analyzing tRNA fragments. In one aspect, the present invention includes Sa method of identifying a subject in need of therapeutic intervention to treat a disease or disease progression comprises characterizing the identity of tRNA fragments. The invention further includes diagnosing identifying or monitoring a disease or condition, a panel of engineered oligonucleotides, a kit for a high-throughput assay, and a method and system for identifying tRNA fragments.

### CROSS-REFERENCE TO RELATED APPLICATION

The present application is entitled to priority under 35 U.S.C. §119(e) to U.S. Provisional Patent Application No. 62/122,711, filed Oct. 28, 2014, which is hereby incorporated by reference in its entirety herein.

### BACKGROUND OF THE INVENTION

Transfer RNAs (tRNAs) are ancient non-coding RNAs (ncRNAs) with a central role in the process of translation of a messenger RNA (mRNA) into an amino acid sequence. As such, tRNAs are present in archaea, bacteria, and eukaryotes. The conventional understanding had been that genomic loci harboring tRNAs produce a single precursor transcript that is processed to produce the mature tRNA. Recent reports suggest that “tRNA fragments” (tRFs) represent a novel and potentially important group of ncRNAs. However, knowledge about their biogenesis, roles and potential functions remains limited and fragmented. Studies with human cell lines have shown that tRNAs can be cleaved at the anticodon loop to produce “tRNA halves” that are (30-35 nts in length) a process that seems to be facilitated by the enzyme, Angiogenin, following induction of stress.

tRNA fragments (tRFs) have also been found to originate from cleavage of either the mature tRNA or the tRNA precursor molecule. In the latter case, RNase Z cleaves the 3′ part of the tRNA precursor as part of the maturation process and the resulting fragment is considered to be a tRF with reported functions. tRFs from the mature tRNA molecule emerge after cleavage at either the D-loop (giving rise to 5′-tRFs) or the T-loop (giving rise to 3′-tRFs with the CCA addition present) and are about 20 nucleotides long. Further investigation into the enzymes responsible for the fragments have been shown to be Dicer-dependent, angiogenin-dependent (cleaving the tRNA at the T-loop) or RNase-Z-dependent (producing 5′-tRFs).

The available evidence indicates that tRFs are not random degradation products. Indeed, some 3′-tRFs have been reported to be loaded onto Argonaute thereby exhibiting behavior akin to a microRNA (miRNA). Also, their involvement in regulation of gene expression affected physiological processes like cell proliferation and cellular responses to DNA damage. 3′-tRF have also been described to emerge in human MT4 T-cells after HIV infection from the host cell. Further supporting the non-random nature of tRFs is the fact that they have been described in mouse, in the yeast S. pombe, in the fruitfly D. melanogaster, in the protozoans G. lamblia and T. thermophile, in the bacterium S. coelicolor, and in the archaeon H. volcanii. Specifically in H. volcanii, four classes of fragments have been described. However, there is limited information detailing these non-coding RNAs (ncRNAs).

Therefore, a need exists for determining the full complement of tRNA fragments, and their regulatory roles and functions in diseased and healthy cells.

### SUMMARY OF THE INVENTION

As described herein, the present invention relates to methods characterizing fragments of tRNAs.

In one aspect, the invention includes a method of identifying a subject in need of therapeutic intervention to treat a disease or condition, disease recurrence, or disease progression comprising isolating fragments of tRNAs from a sample obtained from the subject and characterizing the tRNA fragments and their relative abundance in the sample to identify a signature, wherein when the signature is indicative of a diagnosis of the disease treatment of the subject is recommended.

In another aspect, the invention includes a method of diagnosing, identifying or monitoring breast cancer in a subject in need thereof, the method comprising isolating tRNA fragments from a cell obtained from the subject, hybridizing the tRNA fragments to a panel of oligonucleotides engineered to detect tRNA fragments, analyzing levels of the tRNA fragments present in the cell; wherein a differential in the measured tRNA fragments' levels to the reference is indicative of a diagnosis or identification of breast cancer in the subject, and providing a treatment regimen to the subject dependent on the differential in measured tRNA fragments' levels to the reference.

In yet another aspect, the invention includes a panel of engineered oligonucleotides comprising a mixture of oligonucleotides that are about 15 to about 40 nucleotides in length and capable of hybridizing tRNA fragments, wherein the tRNAs are less than 80 nucleotides in length.

In still another aspect, the invention includes a kit for high-throughput analysis of tRNAs fragments in a sample comprising the panel of engineered oligonucleotides as described herein, hybridization reagents, and tRNA isolation reagents.

In another aspect, the invention includes a method of identifying a cell's tissue of origin to treat a disease or condition, disease recurrence, or disease progression in a subject in need thereof comprising isolating fragments of tRNAs from a cell obtained from the subject, characterizing the identity of the tRNA fragments and their relative abundance in the cell to identify a signature, wherein the signature is indicative of the cell's tissue of origin, and providing a treatment regimen to the subject dependent on the cell's tissue of origin.

In yet another aspect, the invention includes a method for identifying tRNA fragments comprising defining tRNA loci, sequencing a population of RNA fragments, mapping the sequenced RNA fragments to at least one tRNA genomic loci comprising disregarding mapped RNA fragments that differ in sequence from the tRNA genomic loci by at least an insertion, deletion, or replacement of a nucleotide, adding back mapped RNA fragments that are post-transcriptionally modified that differ in sequence from the tRNA genomic loci only at the post-transcriptional modification, excluding mapped RNA fragments that map to locations in the genome outside of the tRNA genomic loci, and disregarding mapped RNA fragments with tRNA intron sequences, and characterizing the mapped RNA fragments.

In still another aspect, the invention includes a system for identifying tRNA fragments according to the method described herein comprising a processor capable of analyzing the tRNA fragments.

In various embodiments of the above aspects or any other aspect of the invention delineated herein, the sample is isolated from a cell, tissue or body fluid obtained from the subject. Examples of body fluid include amniotic fluid, aqueous humour and vitreous humour, bile, blood serum, breast milk, cerebrospinal fluid, cerumen, chyle, chyme, endolymph and perilymph, exudates, feces, female ejaculate, gastric acid, gastric juice, lymph, mucus, pericardial fluid, peritoneal fluid, pleural fluid, pus, rheum, saliva, sebum, serous fluid, semen, smegma, sputum, synovial fluid, sweat, tears, urine, vaginal secretion, and vomit. In one embodiment, the sample is selected from the group consisting of a peripheral blood cell, a tumor cell, a circulating tumor cell, an exosome, a bone marrow cell, a breast cell, a lung cell, and a pancreatic cell.

In another embodiment, the tRNA fragments are isolated by a method selected from the group consisting of size selection, sequencing, and amplification. In yet another embodiment, the step of isolating the tRNA fragments comprises isolating tRNA fragments with a length in the range of about 15 nucleotides to about 80 nucleotides. In still another embodiment, the step of isolating the tRNA fragments comprises isolating tRNA fragments having a predominant length of 16, 17, 26, or 29 nucleotides is indicative of a breast cancer subtype.

In one embodiment, the signature is obtained through sequence-specific methods that preserve at least one terminus of the tRNA fragments. In another embodiment, the signature is obtained by hybridization to a panel of oligonucleotides. In still another embodiment, the tRNA fragments are enriched prior to the hybridization. In yet another embodiment, the oligonucleotide panel comprises at least two or more polynucleotides that selectively hybridize to the tRNA fragments.

In one embodiment, the signature comprises at least one sequence with identifiers selected from the group consisting of SEQ ID NOs: 1-1802 to analyze brain; SEQ ID NOs: 8538-8852 to analzye breast tissue; SEQ ID NOs: 12462-14475 to analyze blood cells; SEQ ID NOs: 24833-25945 to analyze blood cells; SEQ ID NOs: 36100-37466 to analyze pancreatic cancer; SEQ ID NOs: 42349-43721 to analyze prostate tissue; and SEQ ID NOs: 51286-51793 to analyze platelets.

In one embodiment, the signature comprises at least one sequence with identifiers selected from the group consisting of SEQ ID NOs: 11, 18, 19, 28, 31, 34, 43, 51, 59, 83, 189, 194, 209, 268, 305, 306, 307, 316, 320, 398, 404, 611, 632, 653, 696, 751, 768, 816, 817, 860, 869, 870, 871, 920, 921, 925, 951, 960, 967, 989, 1005, 1030, 1133, 1201, 1202, 1223, 1229, 1230, 1231, 1240, 1248, 1298, 1318, 1406, 1412, 1421, 1425, 1453, 1510, 1577, 1582, 1631, 1637, 1645, 1661, 1695, 1727 and 1794 to distinguish Alzheimer's disease brain from normal brain.

In one embodiment, the signature comprises at least one sequence with identifiers selected from the group consisting of SEQ ID NO:8613 and SEQ ID NO: 8823 to distinguish triple negative breast cancer from HER2+ breast cancer.

In one embodiment, the signature comprises at least one sequence with identifiers selected from the group consisting of SEQ ID NOs: 8542, 8543, 8566, 8579, 8582, 8587, 8589, 8590, 8594, 8671-8673, 8707, 8731, 8774-8778, 8803, 8827-8828, 8831-8832, 8837-8838, and 8852 to distinguish triple negative breast cancer from normal.

In one embodiment, the signature comprises at least one sequence with identifiers selected from the group consisting of SEQ ID NOs: 8596, 8601, 8622, 8657, 8664, and 8811 to distinguish triple positive breast cancer from triple negative breast cancer.

In one embodiment, the signature comprises at least one sequence with identifiers selected from the group consisting of SEQ ID NOs: 8582, 8599-8601, 8622-8623, 8634, 8657, 8663-8665, 8676, 8698, 8703-8706, 8718-8720, 8722, 8724, 8738, 8745, 8758, 8761, 8767-8772, and 8840 to distinguish breast cancer from normal tissue.

In one embodiment, the signature comprises at least one sequence with identifiers selected from the group consisting of SEQ ID NOs: 12462, 12463, 12464, 12465, 12466, 12467, 12468, 12469, 12470, 12471, 12472, 12473, 12474, 12475, 12476, 12477, 12478, 12479, 12480, 12481, 12482, 12483, 12484, 12485, 12486, 12487, 12488, 12489, 12490, 12492, 12493, 12494, 12495, 12496, 12497, 12498, 12499, 12500, 12501, 12502, 12503, 12504, 12505, 12506, 12507, 12508, 12509, 12510, 12511, 12512, 12513, 12514, 12515, 12516, 12517, 12518, 12519, 12520, 12522, 12523, 12524, 12525, 12526, 12527, 12529, 12530, 12531, 12532, 12533, 12534, 12536, 12537, 12538, 12540, 12541, 12542, 12543, 12544, 12545, 12546, 12547, 12548, 12549, 12550, 12551, 12552, 12553, 12554, 12555, 12556, 12557, 12558, 12559, 12560, 12561, 12562, 12563, 12564, 12565, 12566, 12567, 12568, 12569, 12570, 12572, 12573, 12574, 12575, 12576, 12577, 12578, 12580, 12581, 12582, 12584, 12585, 12586, 12587, 12588, 12589, 12590, 12591, 12592, 12593, 12594, 12595, 12596, 12597, 12598, 12599, 12600, 12601, 12602, 12603, 12604, 12607, 12608, 12609, 12614, 12615, 12616, 12617, 12618, 12619, 12620, 12621, 12622, 12623, 12624, 12625, 12626, 12627, 12628, 12629, 12631, 12632, 12633, 12634, 12635, 12636, 12637, 12638, 12639, 12640, 12641, 12642, 12643, 12645, 12647, 12648, 12649, 12652, 12653, 12654, 12655, 12657, 12658, 12659, 12660, 12661, 12663, 12664, 12665, 12666, 12667, 12668, 12669, 12670, 12671, 12672, 12674, 12677, 12678, 12679, 12680, 12682, 12684, 12685, 12686, 12687, 12688, 12689, 12690, 12691, 12692, 12693, 12694, 12695, 12696, 12697, 12698, 12699, 12700, 12703, 12704, 12705, 12706, 12708, 12710, 12711, 12712, 12713, 12714, 12715, 12716, 12717, 12718, 12719, 12720, 12721, 12724, 12726, 12727, 12728, 12729, 12730, 12731, 12732, 12733, 12734, 12736, 12738, 12739, 12740, 12741, 12742, 12743, 12744, 12745, 12746, 12747, 12749, 12750, 12751, 12754, 12756, 12758, 12760, 12761, 12763, 12764, 12765, 12766, 12767, 12768, 12769, 12770, 12771, 12773, 12774, 12776, 12777, 12779, 12780, 12781, 12782, 12783, 12785, 12786, 12788, 12789, 12790, 12791, 12792, 12795, 12799, 12800, 12801, 12802, 12803, 12804, 12805, 12806, 12807, 12809, 12811, 12812, 12813, 12814, 12815, 12817, 12818, 12819, 12820, 12821, 12824, 12825, 12826, 12827, 12828, 12829, 12831, 12832, 12833, 12834, 12835, 12836, 12837, 12838, 12840, 12841, 12842, 12843, 12844, 12846, 12847, 12848, 12849, 12850, 12851, 12852, 12853, 12854, 12855, 12856, 12857, 12858, 12859, 12860, 12861, 12864, 12865, 12867, 12868, 12869, 12870, 12871, 12872, 12873, 12874, 12875, 12876, 12877, 12878, 12879, 12880, 12881, 12882, 12883, 12884, 12885, 12886, 12887, 12888, 12889, 12890, 12891, 12892, 12893, 12894, 12895, 12896, 12897, 12899, 12900, 12901, 12902, 12903, 12904, 12905, 12906, 12907, 12909, 12910, 12911, 12912, 12913, 12914, 12916, 12918, 12919, 12920, 12922, 12923, 12924, 12925, 12926, 12927, 12928, 12929, 12930, 12931, 12932, 12933, 12934, 12935, 12936, 12937, 12938, 12939, 12940, 12941, 12942, 12943, 12944, 12946, 12947, 12948, 12949, 12950, 12951, 12954, 12955, 12956, 12957, 12958, 12959, 12960, 12961, 12962, 12963, 12965, 12966, 12967, 12968, 12969, 12970, 12971, 12972, 12973, 12974, 12975, 12978, 12979, 12980, 12981, 12982, 12983, 12984, 12985, 12986, 12987, 12988, 12990, 12991, 12992, 12993, 12994, 12996, 12997, 12998, 12999, 13000, 13001, 13002, 13003, 13004, 13005, 13006, 13007, 13008, 13009, 13011, 13012, 13013, 13014, 13016, 13017, 13018, 13019, 13020, 13021, 13022, 13023, 13024, 13025, 13028, 13029, 13030, 13031, 13033, 13034, 13035, 13036, 13037, 13038, 13039, 13040, 13044, 13045, 13046, 13047, 13049, 13050, 13051, 13052, 13053, 13054, 13055, 13056, 13057, 13058, 13059, 13061, 13063, 13065, 13066, 13067, 13068, 13069, 13070, 13071, 13072, 13073, 13074, 13075, 13076, 13077, 13078, 13079, 13080, 13081, 13082, 13083, 13084, 13085, 13086, 13087, 13088, 13089, 13090, 13091, 13092, 13093, 13094, 13095, 13096, 13097, 13098, 13100, 13101, 13102, 13103, 13104, 13105, 13106, 13107, 13110, 13112, 13113, 13114, 13117, 13118, 13119, 13120, 13121, 13122, 13123, 13124, 13125, 13127, 13128, 13129, 13130, 13131, 13132, 13133, 13134, 13135, 13136, 13137, 13138, 13139, 13140, 13141, 13142, 13143, 13145, 13146, 13148, 13149, 13150, 13151, 13152, 13153, 13154, 13155, 13157, 13158, 13159, 13160, 13161, 13162, 13163, 13164, 13165, 13166, 13167, 13168, 13169, 13170, 13171, 13174, 13175, 13177, 13178, 13179, 13181, 13182, 13183, 13184, 13185, 13186, 13187, 13189, 13190, 13191, 13193, 13195, 13196, 13198, 13199, 13200, 13201, 13202, 13203, 13204, 13205, 13206, 13207, 13208, 13209, 13210, 13211, 13212, 13213, 13214, 13215, 13216, 13217, 13218, 13219, 13221, 13222, 13223, 13225, 13228, 13230, 13231, 13232, 13233, 13234, 13236, 13237, 13238, 13239, 13240, 13241, 13242, 13243, 13245, 13246, 13247, 13248, 13249, 13250, 13251, 13252, 13253, 13255, 13256, 13257, 13258, 13259, 13260, 13261, 13262, 13263, 13264, 13268, 13269, 13270, 13271, 13273, 13274, 13275, 13276, 13277, 13278, 13279, 13280, 13281, 13283, 13285, 13286, 13287, 13288, 13289, 13290, 13292, 13293, 13294, 13295, 13296, 13297, 13298, 13299, 13300, 13301, 13302, 13303, 13304, 13306, 13309, 13310, 13312, 13313, 13314, 13315, 13316, 13317, 13318, 13319, 13320, 13323, 13324, 13325, 13326, 13327, 13328, 13329, 13330, 13331, 13332, 13333, 13334, 13335, 13336, 13337, 13338, 13339, 13340, 13341, 13342, 13343, 13345, 13346, 13347, 13348, 13349, 13350, 13351, 13352, 13353, 13354, 13355, 13357, 13358, 13359, 13360, 13361, 13362, 13363, 13364, 13365, 13366, 13367, 13369, 13370, 13371, 13372, 13373, 13374, 13375, 13376, 13377, 13378, 13379, 13380, 13381, 13382, 13383, 13384, 13385, 13386, 13387, 13388, 13389, 13390, 13391, 13392, 13393, 13394, 13395, 13396, 13397, 13398, 13399, 13400, 13401, 13402, 13403, 13404, 13405, 13406, 13407, 13408, 13409, 13410, 13411, 13412, 13413, 13414, 13415, 13416, 13417, 13421, 13422, 13424, 13426, 13427, 13428, 13429, 13430, 13431, 13432, 13433, 13434, 13436, 13437, 13438, 13439, 13440, 13441, 13442, 13443, 13445, 13446, 13447, 13448, 13449, 13450, 13452, 13453, 13454, 13455, 13456, 13457, 13458, 13459, 13460, 13461, 13462, 13463, 13464, 13465, 13466, 13467, 13468, 13469, 13470, 13471, 13472, 13473, 13474, 13475, 13476, 13477, 13478, 13479, 13480, 13481, 13482, 13484, 13485, 13486, 13488, 13489, 13491, 13492, 13493, 13494, 13495, 13496, 13498, 13500, 13501, 13503, 13504, 13505, 13506, 13507, 13508, 13509, 13510, 13511, 13512, 13513, 13514, 13516, 13517, 13519, 13520, 13522, 13523, 13524, 13525, 13528, 13529, 13530, 13531, 13532, 13533, 13534, 13535, 13536, 13537, 13538, 13539, 13540, 13541, 13542, 13543, 13544, 13545, 13546, 13547, 13548, 13550, 13551, 13552, 13553, 13554, 13556, 13557, 13558, 13559, 13560, 13561, 13562, 13563, 13567, 13568, 13569, 13570, 13571, 13572, 13573, 13574, 13576, 13577, 13578, 13579, 13580, 13581, 13582, 13583, 13584, 13585, 13586, 13587, 13588, 13589, 13590, 13591, 13592, 13593, 13594, 13595, 13596, 13597, 13598, 13599, 13600, 13601, 13602, 13603, 13604, 13605, 13606, 13607, 13608, 13609, 13610, 13611, 13612, 13613, 13614, 13615, 13616, 13617, 13619, 13620, 13621, 13622, 13623, 13624, 13626, 13627, 13628, 13629, 13632, 13633, 13634, 13635, 13636, 13637, 13638, 13639, 13640, 13641, 13642, 13643, 13644, 13645, 13646, 13647, 13648, 13649, 13650, 13651, 13654, 13655, 13656, 13657, 13658, 13659, 13660, 13661, 13662, 13663, 13664, 13665, 13666, 13667, 13668, 13669, 13670, 13671, 13672, 13673, 13674, 13675, 13676, 13677, 13678, 13679, 13680, 13681, 13682, 13683, 13684, 13685, 13687, 13688, 13690, 13691, 13693, 13695, 13696, 13697, 13699, 13700, 13702, 13703, 13704, 13706, 13707, 13708, 13709, 13710, 13711, 13712, 13713, 13714, 13716, 13717, 13718, 13719, 13720, 13721, 13722, 13723, 13724, 13725, 13726, 13727, 13728, 13729, 13730, 13731, 13732, 13733, 13734, 13735, 13737, 13738, 13739, 13740, 13741, 13742, 13743, 13744, 13745, 13746, 13747, 13748, 13749, 13750, 13751, 13752, 13754, 13755, 13756, 13757, 13758, 13759, 13760, 13762, 13763, 13764, 13765, 13766, 13767, 13768, 13769, 13770, 13771, 13772, 13774, 13775, 13776, 13777, 13778, 13779, 13780, 13781, 13782, 13783, 13784, 13785, 13786, 13787, 13788, 13789, 13790, 13792, 13793, 13794, 13795, 13796, 13799, 13801, 13802, 13803, 13804, 13806, 13807, 13808, 13809, 13810, 13811, 13812, 13813, 13815, 13816, 13817, 13818, 13819, 13820, 13821, 13822, 13823, 13824, 13825, 13826, 13827, 13828, 13829, 13830, 13831, 13833, 13834, 13835, 13836, 13837, 13838, 13839, 13841, 13842, 13843, 13844, 13845, 13846, 13849, 13850, 13851, 13852, 13853, 13854, 13855, 13856, 13857, 13858, 13859, 13860, 13861, 13862, 13863, 13864, 13865, 13866, 13868, 13869, 13870, 13871, 13873, 13874, 13875, 13876, 13878, 13879, 13880, 13881, 13882, 13884, 13885, 13887, 13888, 13889, 13890, 13893, 13895, 13896, 13897, 13898, 13899, 13900, 13901, 13902, 13903, 13904, 13905, 13906, 13908, 13909, 13910, 13911, 13912, 13914, 13915, 13916, 13917, 13919, 13920, 13921, 13922, 13923, 13924, 13925, 13926, 13928, 13929, 13930, 13931, 13932, 13933, 13934, 13935, 13936, 13937, 13938, 13939, 13940, 13941, 13942, 13944, 13945, 13946, 13948, 13950, 13952, 13953, 13954, 13955, 13956, 13960, 13961, 13962, 13963, 13964, 13965, 13966, 13967, 13968, 13970, 13971, 13972, 13973, 13974, 13975, 13976, 13977, 13978, 13979, 13980, 13982, 13983, 13984, 13985, 13986, 13987, 13988, 13989, 13990, 13991, 13992, 13993, 13994, 13995, 13996, 13997, 13998, 13999, 14000, 14001, 14002, 14003, 14004, 14005, 14006, 14007, 14008, 14010, 14011, 14012, 14013, 14014, 14015, 14016, 14017, 14018, 14019, 14020, 14021, 14022, 14023, 14024, 14025, 14026, 14027, 14028, 14030, 14031, 14032, 14034, 14035, 14037, 14038, 14039, 14040, 14041, 14042, 14043, 14044, 14045, 14046, 14047, 14048, 14049, 14050, 14051, 14052, 14053, 14055, 14059, 14060, 14061, 14062, 14064, 14065, 14067, 14068, 14069, 14070, 14071, 14072, 14073, 14074, 14075, 14076, 14077, 14078, 14079, 14080, 14082, 14084, 14085, 14086, 14088, 14089, 14090, 14092, 14093, 14095, 14096, 14097, 14098, 14099, 14100, 14103, 14104, 14105, 14108, 14109, 14110, 14111, 14112, 14113, 14116, 14117, 14118, 14119, 14121, 14122, 14123, 14124, 14125, 14126, 14127, 14128, 14129, 14130, 14131, 14132, 14133, 14135, 14136, 14137, 14139, 14141, 14142, 14143, 14144, 14145, 14146, 14147, 14148, 14151, 14152, 14153, 14154, 14155, 14156, 14157, 14158, 14159, 14160, 14161, 14162, 14163, 14166, 14167, 14168, 14169, 14170, 14171, 14172, 14173, 14175, 14176, 14177, 14178, 14179, 14180, 14181, 14182, 14183, 14185, 14186, 14187, 14188, 14190, 14191, 14192, 14193, 14194, 14195, 14197, 14198, 14199, 14201, 14204, 14205, 14207, 14208, 14212, 14213, 14215, 14216, 14217, 14218, 14219, 14222, 14223, 14224, 14225, 14226, 14227, 14228, 14229, 14230, 14231, 14232, 14233, 14234, 14235, 14236, 14237, 14238, 14239, 14240, 14241, 14242, 14243, 14244, 14245, 14246, 14247, 14248, 14249, 14250, 14251, 14252, 14253, 14254, 14255, 14256, 14257, 14258, 14259, 14260, 14261, 14262, 14263, 14265, 14266, 14267, 14268, 14271, 14273, 14274, 14276, 14280, 14281, 14282, 14283, 14284, 14285, 14287, 14288, 14290, 14292, 14293, 14294, 14295, 14296, 14297, 14298, 14299, 14300, 14301, 14302, 14303, 14304, 14305, 14306, 14307, 14308, 14309, 14310, 14311, 14313, 14314, 14315, 14316, 14317, 14320, 14321, 14322, 14323, 14324, 14325, 14326, 14328, 14329, 14330, 14331, 14332, 14333, 14334, 14335, 14336, 14338, 14339, 14340, 14342, 14343, 14344, 14346, 14347, 14348, 14349, 14350, 14351, 14353, 14354, 14355, 14356, 14357, 14358, 14359, 14360, 14361, 14363, 14365, 14366, 14367, 14368, 14369, 14370, 14371, 14372, 14373, 14374, 14375, 14376, 14377, 14378, 14379, 14380, 14382, 14383, 14384, 14385, 14386, 14389, 14390, 14391, 14392, 14393, 14394, 14395, 14396, 14397, 14399, 14400, 14401, 14402, 14403, 14404, 14405, 14406, 14407, 14408, 14409, 14410, 14411, 14412, 14413, 14415, 14416, 14417, 14418, 14419, 14420, 14421, 14422, 14424, 14427, 14428, 14429, 14430, 14432, 14434, 14435, 14436, 14437, 14438, 14440, 14441, 14442, 14443, 14444, 14445, 14446, 14447, 14448, 14450, 14451, 14452, 14453, 14454, 14455, 14456, 14457, 14458, 14459, 14460, 14461, 14463, 14465, 14467, 14469, 14470, 14471, 14473, 14475 to distinguish chronic lymphocytic leukemia from normal B-cells.

In one embodiment, the signature comprises at least one sequence with identifiers selected from the group consisting of SEQ ID NOs: 24995-24996, 25025, 25031, 25033, 25087-25091, 25093-25094, 25128, 25150, 25161-25162, 25165, 25182, 25219-25220, 25230, 25277-25278, 25284, 25316, 25356-25357, 25359-25360, 25363-25364, 25397-25398, 25415, 25424, 25432, 25480, 25484-25486, 25498-25499, 25505, 25524, 25550-25552, 25570, 25580, 25583, 25609-25610, 25619, 25646-25647, 25685-25687, 25691, 25714, 25720, 25727-25728, 25731, 25741, 25746-25747, 25846-25847, 25868, 25882, 25904, 25908-25912, and 25914-25915 to distinguish B-cells from breast cells.

In one embodiment, the signature comprises at least one sequence with identifiers selected from the group consisting of SEQ ID NOs: 24880-24883, 24896-24897, 24959-24963, 24965, 24973, 25006, 25027, 25052, 25054, 25102-25103, 25110-25111, 25118, 25123, 25150, 25152-25153, 25183-25184, 25188, 25198, 25202, 25204-25206, 25210, 25212-25214, 25224-25225, 25245, 25252-25254, 25257, 25259-25261, 25270, 25273, 25286, 25294, 25296, 25313-25314, 25334, 25416, 25425, 25449-25450, 25454, 25476-25478, 25583, 25609-25612, 25665, 25667, 25705, 25714, 25786, 25894, and 25896-25897 to distinguish B-cells from white people from B-cells from black people.

In one embodiment, the signature comprises at least one sequence with identifiers selected from the group consisting of SEQ ID NO: 24881, 24926, 24952, 24981, 24990, 24995, 24998, 25010, 25047, 25051, 25075, 25101-25102, 2511 25111, 25118, 25121, 25149, 25211, 25218, 25238, 25309, 25359, 25373, 25376, 25386-25387, 25402, 25410, 25415-25416, 25420-25421, 25468, 25474, 25476, 25484-25487, 25493, 25524, 25536, 25560, 25596, 25604, 25620, 25631, 25651, 25662, 25664, 25714, 25723, 25803, 25829, 25850-25851, 25886-25887, 25898, 25902-25903, 25905, 25914, 25921, 25923, 25937 to distinguish B-cells from men from B-cells from women.

In one embodiment, the signature comprises at least one sequence with identifiers selected from the group consisting of SEQ ID NOs: 36100, 36101, 36105, 36107, 36111, 36112, 36114, 36115, 36116, 36119, 36120, 36121, 36122, 36123, 36139, 36143, 36146, 36147, 36148, 36149, 36155, 36156, 36157, 36163, 36171, 36173, 36176, 36177, 36178, 36179, 36180, 36181, 36182, 36183, 36188, 36189, 36194, 36197, 36200, 36203, 36204, 36215, 36217, 36218, 36219, 36222, 36223, 36227, 36228, 36230, 36231, 36234, 36238, 36239, 36240, 36241, 36242, 36243, 36246, 36248, 36252, 36254, 36262, 36265, 36266, 36269, 36270, 36271, 36272, 36273, 36276, 36278, 36279, 36282, 36285, 36287, 36288, 36289, 36293, 36294, 36295, 36296, 36297, 36298, 36299, 36303, 36304, 36305, 36306, 36307, 36308, 36313, 36319, 36320, 36322, 36323, 36326, 36327, 36331, 36332, 36333, 36335, 36336, 36338, 36339, 36341, 36342, 36344, 36347, 36355, 36356, 36357, 36372, 36373, 36374, 36375, 36376, 36378, 36378, 36384, 36387, 36391, 36392, 36395, 36397, 36399, 36400, 36401, 36405, 36406, 36408, 36409, 36428, 36429, 36430, 36431, 36432, 36433, 36435, 36436, 36437, 36444, 36450, 36451, 36452, 36453, 36455, 36456, 36457, 36460, 36461, 36462, 36463, 36464, 36465, 36466, 36467, 36468, 36469, 36470, 36471, 36472, 36478, 36485, 36490, 36491, 36498, 36499, 36504, 36505, 36506, 36507, 36508, 36509, 36510, 36511, 36512, 36513, 36517, 36520, 36521, 36523, 36524, 36529, 36530, 36533, 36534, 36535, 36538, 36539, 36541, 36542, 36543, 36544, 36545, 36546, 36547, 36550, 36553, 36554, 36561, 36562, 36572, 36573, 36574, 36575, 36578, 36579, 36580, 36581, 36582, 36584, 36586, 36589, 36590, 36591, 36593, 36594, 36597, 36599, 36600, 36601, 36607, 36608, 36609, 36610, 36611, 36612, 36614, 36615, 36616, 36617, 36618, 36619, 36620, 36621, 36627, 36628, 36629, 36637, 36638, 36639, 36640, 36641, 36642, 36643, 36644, 36645, 36646, 36647, 36649, 36650, 36658, 36665, 36669, 36670, 36671, 36673, 36674, 36675, 36676, 36677, 36678, 36679, 36680, 36682, 36683, 36684, 36689, 36690, 36691, 36692, 36693, 36694, 36695, 36696, 36697, 36698, 36701, 36702, 36703, 36705, 36706, 36707, 36708, 36709, 36710, 36711, 36712, 36714, 36715, 36716, 36718, 36719, 36720, 36721, 36722, 36726, 36727, 36728, 36729, 36730, 36731, 36732, 36733, 36734, 36735, 36738, 36739, 36741, 36742, 3674 5, 36746, 36747, 36749, 36751, 36754, 36755, 36756, 36757, 36759, 36760, 36761, 36762, 36763, 36764, 36765, 36768, 36769, 36770, 36771, 36772, 36775, 36776, 36777, 36778, 36788, 36789, 36793, 36794, 36796, 36797, 36798, 36799, 36800, 36803, 36805, 36806, 36809, 36810, 36812, 36814, 36817, 36825, 36826, 36827, 36829, 36830, 36831, 36832, 36834, 36835, 36838, 36839, 36841, 36844, 36846, 36848, 36849, 36851, 36854, 36855, 36857, 36859, 36860, 36861, 36862, 36863, 36864, 36868, 36869, 36871, 36872, 36877, 36878, 36879, 36880, 36881, 36883, 36884, 36885, 36886, 36887, 36889, 36890, 36891, 36892, 36895, 36897, 36901, 36902, 36903, 36904, 36905, 36907, 36909, 36910, 36911, 36913, 36914, 36915, 36916, 36917, 36918, 36919, 36925, 36931, 36938, 36939, 36941, 36942, 36945, 36946, 36948, 36952, 36953, 36955, 36956, 36957, 36958, 36961, 36963, 36964, 36965, 36967, 36968, 36973, 36976, 36977, 36978, 36979, 36980, 36981, 36982, 36983, 36985, 36988, 36989, 36990, 36991, 36992, 36997, 36998, 36999, 37001, 37004, 37005, 37008, 37009, 37012, 37013, 37014, 37021, 37022, 37023, 37024, 37025, 37026, 37029, 37032, 37033, 37036, 37039, 37044, 37046, 37048, 37049, 37050, 37051, 37054, 37055, 37056, 37057, 37058, 37059, 37060, 37063, 37065, 37066, 37075, 37077, 37078, 37079, 37080, 37081, 37083, 37087, 37088, 37089, 37090, 37091, 37094, 37095, 37099, 37100, 37101, 37110, 37115, 37116, 37117, 37119, 37120, 37121, 37123, 37124, 37125, 37127, 37132, 37133, 37134, 37135, 37137, 37138, 37139, 37141, 37142, 37143, 37144, 37145, 37146, 37149, 37150, 37151, 37152, 37155, 37157, 37160, 37161, 37162, 37163, 37164, 37165, 37166, 37167, 37168, 37169, 37171, 37174, 37175, 37177, 37178, 37181, 37182, 37183, 37184, 37185, 37187, 37193, 37194, 37195, 37196, 37197, 37198, 37199, 37201, 37202, 37203, 37206, 37207, 37208, 37209, 37211, 37213, 37214, 37216, 37217, 37226, 37227, 37228, 37229, 37230, 37231, 37234, 37235, 37237, 37244, 37245, 37247, 37248, 37249, 37251, 37253, 37254, 37255, 37261, 37262, 37265, 37271, 37272, 37273, 37274, 37278, 37279, 37283, 37303, 37304, 37305, 37306, 37307, 37308, 37312, 37316, 37319, 37321, 37323, 37324, 37325, 37326, 37327, 37334, 37335, 37336, 37337, 37338, 37339, 37340, 37341, 37342, 37348, 37356, 37363, 37365, 37368, 37369, 37370, 37372, 37374, 37375, 37376, 37382, 37383, 37385, 37386, 37388, 37391, 37394, 37395, 37398, 37400, 37401, 37402, 37403, 37404, 37405, 37407, 37408, 37410, 37419, 37420, 37422, 37423, 37424, 37425, 37426, 37429, 37430, 37431, 37432, 37433, 37445, 37446, 37448, 37449, 37453, 37454, 37456, 37461, 37462, 37463, 37464, and 37466 to distinguish normal pancreas from pancreatic cancer.

In one embodiment, the signature comprises at least one sequence with identifiers selected from the group consisting of SEQ ID NOs: 51377-51378, 51406, 51438, 51496, 51565, 51691, 51699, 51736-51737, 51745, and 51759 to distinguish platelets from people with a propensity to clot vs. platelets from people with a propensity to hemorrhage.

In one embodiment, the signature comprises at least one sequence with identifiers selected from the group consisting of SEQ ID NOs: 42434, 42520, 42537, 42577, 42751, 42979, 43019, 43090, 43128, 43156, 43310, 43352, 43398, 43426, 43437 to distinguish normal prostate from prostate cancer.

In one embodiment, the step of characterizing the tRNA fragments comprises at least one assessment selected from the group consisting of sequencing the tRNA fragments, measuring overall abundance of one of the tRNA fragments mapped to the genome, measuring a relative abundance of the one tRNA fragment to a reference, assessing a length of the one tRNA fragment, identifying starting and ending points of the one tRNA fragment, identifying genomic origin of the one tRNA fragment, and identifying a terminal modification of the one tRNA fragment. In another embodiment, the step of characterizing the mapped RNA fragments comprises at least one assessment selected from the group consisting of identifying one or more of the mapped RNA fragments in a population, measuring an overall abundance of one or more of the mapped RNA fragments, measuring a relative abundance of one or more of the mapped RNA fragments to a reference, assessing a length of one or more of the mapped RNA fragments, identifying starting and ending points of one or more of the mapped RNA fragments, identifying genomic origin of one or more of the mapped RNA fragments, and identifying a terminal modification of one or more of the mapped RNA fragments.

In one embodiment, the disease or condition, disease recurrence, or disease progression is selected from the group consisting of a cancer, and genetically predisposed disease or condition.

In another embodiment, the tRNA genomic loci comprise mitochondrial tRNA sequences from the mitochondrial genome, nuclear tRNA sequences from the nuclear genome, and mitochondria tRNA sequences from the nuclear genome. In another embodiment, the mapped RNA fragments post-transcriptionally modified comprises at least one modified with a CCA trinucleotide at a 3′ end.

### BRIEF DESCRIPTION OF THE DRAWINGS

FIG. 1 is an illustration showing the typical tRNA cloverleaf secondary structure and the five categories of tRNA fragments that are known currently as a result of the discovery that is discussed herein. In practice, a typical tRNA may produce more than just 11 distinct fragments or fewer.

FIG. 2 shows breast cancer (BRCA) subgroups and receptor expressions. In HER2 negative Luminal-type, the level of Ki67, an indicator of cell proliferation rate, is used to further classify Luminal A (low level) and Luminal B (high level).

FIGS. 3A-3D are graphs showing atypical tRNA fragment lengths in the 452 analyzed lymphoblastoid cell line (LCL) samples. Shown are the length distributions for all fragments supported by reads that land solely in the tRNA space and can be positioned anywhere along the length of a mature tRNA. FIG. 3A shows the length distribution for internal tRNA fragments (i-tRFs) only. FIG. 3B shows the length distribution for “+1” tRNA fragments only. FIG. 3C shows the length distribution for “CCA-ending” tRNA fragments. FIG. 3D shows the length distribution for all these tRNA fragments combined. See also text for a detailed explanation of these three shown regions. Error bars capture standard error across the 452 samples.

FIG. 4A-4D are graphs showing atypical tRNA fragment lengths in the 311 analyzed breast samples from The Cancer Genome Atlas repository. Shown are the length distributions for all fragments supported by reads that land solely in the tRNA space and can be positioned anywhere along the length of a mature tRNA. FIG. 4A shows the length distribution for internal tRNA fragments (i-tRFs) only. FIG. 4B shows the length distribution for “+1” tRNA fragments only. FIG. 4C shows the length distribution for “CCA-ending” tRNA fragments. FIG. 4D shows the length distribution for all these tRNA fragments combined. Error bars capture standard error across the 311 samples. Note the right most label of the X-axis: it was labeled in such a way to indicate the possibility that some of the observed 30-mers have arisen from longer length fragments, as discussed elsewhere herein.

FIG. 5A-5B are 3D graphs showing the distribution of starting position and lengths for internal tRNA fragments (i-tRFs), their span and lengths in the LCL (A) and BRCA (B) datasets. The positions are numbered with reference to the +1 position of the mature tRNA. The representative positions for the D- and T-loops as well as for the anticodon loop are highlighted with green boxes. The coloring of each bar is proportional to the relative abundance of each length of the fragments starting at that specific position as indicated by the respective color-key below each graph. The thickness of the projections on the right wall of the graph is proportional to the number of fragments spanning the specific position. For the LCL dataset, only the top 50% most expressed internal fragments are shown.

FIG. 6A-6B are graphs showing the relative abundance of fragments from nuclear and mitochondrial tRNAs as a function of their length for the LCLs samples (A) and the BRCA samples (B). Error bars capture the standard error across the analyzed samples. The statistically significant difference in abundance (P-value; Mann-Whitney U-test) is indicated for two cases in each dataset.

FIGS. 7A-7B show heatmaps of the Pearson correlation coefficient for statistically significant fragments. FIG. 7A shows tRNA fragments that arise from the nuclear AspGTC (trna10 on chromosome 12) anticodon in the LCL dataset. FIG. 7B shows tRNA fragments that arise from the mitochondrial GluTTC anticodon in the BRCA dataset. Several mini-clusters are evident in each heatmap: however, there was correlation across the mini-clusters of the same tRNA (see text for a detailed explanation). Orange-colored labels mark the i-tRFs.

FIGS. 8A-8D show atypical tRNA fragment lengths in normal and tumor breast samples. FIG. 8A shows length distribution for the internal tRNA fragments (i-tRFs).

FIG. 8B shows length distributions for fragments in the “+1” region. FIG. 8C shows length distribution for “CCA-ending” fragments. FIG. 8D shows length distributions for the all the fragments. Green curve: normal sample fragments. Red curve: tumor sample fragments.

FIGS. 9A-9B show common tRNA fragments have tissue- and tissue-state specific abundances. FIG. 9A shows that a principal components analysis (PCA) of the abundance levels of the ˜200 tRNA fragments, which were common to female LCL samples and to normal breast samples, can distinguish between the two groups. FIG. 9B shows that a partial Least Squares-Discriminant Analysis (PLS-DA) of the abundance levels of the 437 tRNA fragments found in normal breast and breast cancer samples can distinguish between the two groups.

FIGS. 10A-10C show race-dependent expression profiles for statistically significant tRNA fragments. FIG. 10A shows a PCA of fragment expression in LCLs. The CEU population (white) is represented by the yellow points whereas the YRI population (black) is represented by the magenta points. Both men and women from each of the two populations were included in this analysis. FIG. 10B shows a PLS-DA on the tRNA fragments in the 78 triple-negative-breast-cancer samples. The yellow points represent white patients where the magenta dots represent black patients. FIG. 10C shows relative abundances of CCA-ending 18-mers and 33-mers for the CEU and YRI samples. The differences for both 18-mers and 36-mers were statistically significant as indicated by the asterisks (p-val≦10⁻⁴ using Student's t-test). Error bars capture the standard error of the relative abundance of each type of fragments for n=93 (CEU) and n=95 (YRI) samples.

FIGS. 11A-11C show differences in the abundance of tRNA fragments between men and women. FIG. 11A shows a detail from the length distributions for YRI men and women for internal fragments. FIG. 11B shows a detail from the length distributions for TSI men and women for CCA-ending fragments. FIG. 11C shows a PLS-DA graph of TSI men and TSI women showing a trend for gender-specific tRNA profiles.

FIGS. 12A-12D show differences in the tRNA profiles among normal and different disease states. FIG. 12A shows a PLS-DA for the discrimination of normal against triple positive samples. FIG. 12B shows a PLS-DA graph for the discrimination of normal against triple negative samples. FIG. 12C shows PLS-DA discriminated between the triple positive and triple negative subtypes. FIG. 12D shows that the fragments that were important for each separation can be used to identify disease subtype-specific abundance changes. The number of fragments with higher (↑) or lower (←) expression is indicated next to each arrow. Each arrow represents a comparison between two groups: the start of the arrow indicates the “control” group compared to which the fragments in the “target” group (end of arrow) have altered expression.

FIG. 13 is a graph showing differential Ago-loading of tRNA fragments in three breast cancer model cell lines using only fragments with lengths<=30 nucleotides (nts). Note the difference in lengths of the Ago-loaded fragments across the three cell lines.

FIG. 14A-14B are graphs showing the experimental verification of internal fragments in breast samples and breast model cell lines. (A): Quantification of the i-tRF from the nuclear AspGTC anticodon in 11 breast tumor and 11 adjacent normal breast samples. N.D.: not determined; in this case, the fragment's expression was too low to be detected. Astericks indicate statistically significant changes in abundance (p-val<0.01; Student's t-test) between the tumor and adjacent normal tissue of the same subject. In all cases there were n=3 repetitions of the experiments. Error bars show the standard deviation. (B): Quantification of the i-tRF from the nuclear GlyTCC anticodon in eight different normal and breast cancer cell lines using an assay based on the FIREPLEX® (Firefly BioWorks, Boston, Mass.) method. Column height represents the average expression value and error bars the standard deviation of at least 10 independent measurements in each sample. On the right hand-side of (A) and (B), the tested fragment is highlighted. The anticodon triplet is indicated by the black rectangle. The genomic coordinates of the depicted AspGTC tRNA are from 125424264 to 125424193, inclusive, on chromosome 12, wherease for the depicted GlyTCC tRNA are from 8124866 to 8124937, inclusive, on chromosome 17. ER: Estrogen Receptor, PR: Progesterone Receptor, HER2: Human Epidermal Growth Factor Receptor 2.

FIG. 15 is a graph showing nuclear AspGTC tRNA as an example of the diversity of fragments that can arise from the same tRNA sequence.

### DETAILED DESCRIPTION OF THE INVENTION

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or within 10%, 9%, 8%, 7%, 6%, 5%, 4%, 3%, 2%, 1%, 0.5%, 0.1%, 0.05%, or 0.01% of the specified value, as such variations are appropriate to perform the disclosed methods. Unless otherwise clear from context, all numerical values provided herein are modified by the term about.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “complementary sequence” or “complement” is meant a nucleic acid base sequence that can form a double-stranded structure by matching base pairs to another polynucleotide sequence. Base pairing occurs through the formation of hydrogen bonds, which may be Watson-Crick, Hoogsteen or reversed Hoogsteen hydrogen bonding, between complementary nucleobases. For example, adenine and thymine are complementary nucleobases that pair through the formation of hydrogen bonds.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

The term “cancer” as used herein is defined as disease characterized by the rapid and uncontrolled growth of aberrant cells. Cancer cells can spread locally or through the bloodstream and lymphatic system to other parts of the body. Examples of various cancers include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like.

“Detect” refers to identifying the presence, absence or amount of the biomarker to be detected.

The phrase “differentially present” refers to differences in the quantity and/or the frequency of a biomarker present in a sample taken from subjects having a disease as compared to a control subject. A biomarker can be differentially present in terms of quantity, frequency or both. A polypeptide or polynucleotide is differentially present between two samples if the amount or frequency of the polypeptide or polynucleotide in one sample is statistically significantly different (either higher or lower) from the amount of the polypeptide or polynucleotide in the other sample, such as reference or control samples. Alternatively or additionally, a polypeptide or polynucleotide is differentially present between two sets of samples if the amount or frequency of the polypeptide or polynucleotide in samples of the first set, such as diseased subjects' samples, is statistically significantly (either higher or lower) from the amount of the polypeptide or polynucleotide in samples of the second set, such reference or control samples. A biomarker that is present in one sample, but undetectable in another sample is differentially present.

A “disease” is a state of health of an animal wherein the animal cannot maintain homeostasis, and wherein if the disease is not ameliorated then the animal's health continues to deteriorate. A “disease subtype” is a state of health of an animal wherein animals with the disease manifest different clinical features or symptoms. For example, Alzheimer's disease includes at least three subtypes, inflammatory, non-inflammatory, and cortical.

A “disorder” as used herein, is used interchangeably with “condition,” and refers to a state of health in an animal, wherein the animal is able to maintain homeostasis, but in which the animal's state of health is less favorable than it would be in the absence of the disorder. Left untreated, a disorder does not necessarily cause a further decrease in the animal's state of health.

By “effective amount” is meant the amount required to reduce or improve at least one symptom of a disease relative to an untreated patient. The effective amount of active compound(s) used to practice the present invention for therapeutic treatment of a disease varies depending upon the manner of administration, the age, body weight, and general health of the subject.

As used herein “endogenous” refers to any material from or produced inside an organism, cell, tissue or system.

The term “expression” as used herein is defined as the transcription and/or translation of a particular nucleotide sequence driven by its promoter.

By “fragment” is meant a portion of a polynucleotide or nucleic acid molecule. This portion contains, preferably, at least 10%, 20%, 30%, 40%, 50%, 60%, 70%, 80%, 90%/a, 91%, 92%, 93%, 94%, 95%, 96%, 97%, 98%, or 99% of the entire length of the reference nucleic acids. A fragment may contain 10, 20, 30, 40, 50, 60, 70, 80, 90, or 100, 200, 300, 400, 500, 600, 700, 800, 900, 1000, 1500, 2000 or 2500 (and any integer value in between) nucleotides. The fragment, as applied to a nucleic acid molecule, refers to a subsequence of a larger nucleic acid. The fragment can be an autonomous and functional molecule. A fragment may contain modifications at neither, one or both of its termini. A modification can include but is not limited to a phosphate, a cyclic phosphate, a hydroxyl, and an amino acid. A “fragment” of a nucleic acid molecule may be at least about 15 nucleotides in length; for example, at least about 50 nucleotides to about 100 nucleotides; at least about 100 to about 500 nucleotides, at least about 500 to about 1000 nucleotides, at least about 1000 nucleotides to about 1500 nucleotides; or about 1500 nucleotides to about 2500 nucleotides; or about 2500 nucleotides (and any integer value in between).

“Similar” refers to the sequence similarity or sequence identity between two polypeptides or between two nucleic acid molecules. When a position in both of the two compared sequences is occupied by the same base or amino acid monomer subunit, e.g., if a position in each of two DNA molecules is occupied by adenine, then the molecules are similar at that position. The percent of similarity between two sequences is a function of the number of matching or similar positions shared by the two sequences divided by the number of positions compared X 100. For example, if 6 of 10 of the positions in two sequences are matched or similar then the two sequences are 60% similar. By way of example, the DNA sequences ATTGCC and TATGGC share 50% similarity. Generally, a comparison is made when two sequences are aligned in a way that maximizes their similarity.

As used herein, the term “inhibit” is meant to refer to a decrease in biological state. For example, the term “inhibit” may be construed to refer to the ability to negatively regulate the expression, stability or activity of a protein, including but not limited to transcription of a protein mRNA, stability of a protein mRNA, translation of a protein mRNA, stability of a protein polypeptide, a protein post-translational modifications, a protein activity, a protein signaling pathway or any combination thereof.

Further, the term “inhibit” may be construed to refer to the ability to negatively affect the expression, stability or activity of a miRNA, wherein such inhibition of the miRNA may result in the modulation of a gene, a protein's mRNA abundance, the stability of a protein's mRNA, the translation of a protein's mRNA, the stability of a protein, the post-translational modifications of a protein, and/or the activity of a protein.

“Instructional material,” as that term is used herein, includes a publication, a recording, a diagram, or any other medium of expression that may be used to communicate the usefulness of the compounds of the invention. In some instances, the instructional material may be part of a kit useful for effecting alleviating or treating the various diseases or conditions recited herein. Optionally, or alternately, the instructional material may describe one or more methods of alleviating the diseases or conditions in a cell or a tissue of a mammal. The instructional material of the kit may, for example, be affixed to a container that contains the compounds of the invention or be shipped together with a container that contains the compounds. Alternatively, the instructional material may be shipped separately from the container with the intention that the recipient uses the instructional material and the compound cooperatively. For example, the instructional material is for use of a kit; instructions for use of the compound; or instructions for use of a formulation of the compound.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “mitochondrial tRNAs” is used to refer to tRNAs encoded in the mitochondrial genome. The term “nuclear tRNAs” is used to refer to tRNAs encoded in the nuclear genome. The distinction of the origin of the DNA precursor template may not be entirely accurate from a biological standpoint: as it was recently reported, the nuclear genome contains numerous full-length lookalikes of mitochondrial tRNAs. It is currently unclear whether these nuclear lookalike sequences are transcribed or whether they act as tRNAs; thus, special consideration is needed to discard sequencing reads that may map to those lookalikes and to the tRNA space, which are defined elsewhere herein.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

By “isolated polynucleotide” is meant a nucleic acid (e.g., a DNA or an RNA) that is free of the genes which, in the naturally-occurring genome of the organism from which the nucleic acid molecule of the invention is derived, flank the gene. The term therefore includes, for example, a recombinant DNA that is incorporated into a vector, into an autonomously replicating plasmid or virus; or into the genomic DNA of a prokaryote or eukaryote; or that exists as a separate molecule (for example, a tRNA, cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

The term “oligonucleotide panel” or “panel of oligonucleotides” refers to a collection of one or more oligonucleotides that may be used to identify DNA (e.g. genomic segments comprising a specific sequence, DNA sequences bound by particular protein, etc.) or RNA (e.g. mRNAs, microRNAs, tRNAs, etc.) through hybridization of complementary regions between the oligonucleotides and the DNA or RNA. If the sought molecule is RNA, it is commonly converted to DNA through a reverse transcription step). The oligonucleotides may include complementary sequences to known DNA or known RNA sequences. The oligonucleotides may be engineered to be between about 5 nucleotides to about 40 nucleotides, or about 5 nucleotides to about 30 nucleotides, or about 5 nucleotides to about 20 nucleotides, or about 5 nucleotides to about 15 nucleotides in length. The term “oligonucleotide panel” or “panel of oligonucleotides” could also refer to a system and accompanying collection of reagents that, in addition to being able to hybridize to molecules containing a complementary sequence, can also ensure that the identified molecule's 3′ terminus matches precisely the 3′ terminus of the sought molecule, or that the identified molecule's 5′ terminus matches precisely the 5′ terminus of the sought molecule, or both: this ability is unlike what can be achieved by conventional assays such as e.g. Affymetrix chips and methods (e.g. “dumbbell-PCR”) and systems (e.g. the Fireplex system of Firefly BioWorks) that can achieve this are now beginning to be available.

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

The term “overexpressed” tumor antigen or “overexpression” of the tumor antigen is intended to indicate an abnormally high level of expression of the tumor antigen in a cell from a disease area like a solid tumor within a specific tissue or organ of the patient relative to the level of expression in a normal cell from that tissue or organ. Patients having solid tumors or a hematological malignancy characterized by overexpression of the tumor antigen can be determined by standard assays known in the art. The term “underexpressed” tumor antigen or “underexpression” of the tumor antigen is completely analogous.

The term “overexpressed” tumor promoter or “overexpression” of the tumor promoter is intended to indicate an abnormally high level of expression of the tumor promoter RNA or protein in a cell from a disease area like a solid tumor within a specific tissue or organ of the patient relative to the level of expression in a normal cell from that tissue or organ. Patients having solid tumors or a hematological malignancy characterized by overexpression of the tumor promoter can be determined by standard assays known in the art. The term “underexpressed” tumor promoter or “underexpression” of the tumor promoter is completely analogous.

The term “overexpressed” tumor suppressor or “overexpression” of the tumor suppressor is intended to indicate an abnormally high level of expression of the tumor suppressor RNA or protein in a cell from a specific area within a specific tissue or organ of an individual relative to the level of expression under typical circumstances in a cell from that tissue or organ. Individuals having characteristic overexpression of the tumor suppressor can be determined by standard assays known in the art. The term “underexpressed” tumor suppressor or “underexpression” of the tumor suppressor is completely analogous.

The terms “patient,” “subject,” “individual,” and the like are used interchangeably herein, and refer to a human or non-human mammal, or cells thereof whether in vitro or in situ, amenable to the methods described herein. Non-human mammals include, for example, livestock and pets, such as ovine, bovine, porcine, canine, feline and murine mammals. The term “subject” is intended to include living organisms in which an immune response can be elicited (e.g., mammals). Examples of subjects include humans, dogs, cats, mice, rats, and transgenic species thereof. In certain non-limiting embodiments, the patient, subject or individual is a human.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which may be hydrolyzed into the monomeric “nucleotides.” The monomeric nucleotides may be hydrolyzed into nucleosides. As used herein polynucleotides include, but are not limited to, all nucleic acid sequences that are obtained by any means available in the art, including, without limitation, recombinant means, i.e., the cloning of nucleic acid sequences from a recombinant library or a cell genome, using ordinary cloning technology and PCR™, and the like, and by synthetic means. The following abbreviations for the commonly occurring nucleic acid bases are used. “A” refers to adenosine, “C” refers to cytosine, “G” refers to guanosine, “T” refers to thymidine, and “U” refers to uridine. The term “RNA” as used herein is defined as ribonucleic acid. The term “recombinant DNA” as used herein is defined as DNA produced by joining pieces of DNA from different sources.

As used herein, the terms “prevent,” “preventing,” “prevention,” and the like refer to reducing the probability of developing a disease or condition in a subject, who does not have, but is at risk of or susceptible to developing a disease or condition.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The terms “purified,” or “biologically pure” refer to material that is free to varying degrees from components which normally accompany it as found in its native state. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

A “recyclable tRNA” refers to a tRNA that is aminoacylated and can be repeatedly reaminoacylated with an amino acid (e.g., an unnatural amino acid) for the incorporation of the amino acid (e.g., the unnatural amino acid) into one or more polypeptide chains during translation.

By “reduces” or “decreases” is meant a negative alteration of at least 10%, 25%, 50%, 75%, or 100/%.

By “reference” is meant a standard or control. A “reference” is also a defined standard or control used as a basis for comparison.

As used herein, “relative abundance” refers to the ratio of the quantities of two or more molecules of interest (e.g. tRNAs, tRNA fragments, miRNAs, etc.) present in a sample. The relative abundance of two or more molecules of interest in a given sample may differ from the relative abundance of the same two or more molecules in a second sample.

As used herein, “sample” or “biological sample” refers to anything, which may contain the biomarker (e.g., polypeptide, polynucleotide, or fragment thereof) for which a biomarker assay is desired. The sample may be a biological sample, such as a biological fluid or a biological tissue. In one embodiment, a biological sample is a tissue sample including pulmonary vascular cells. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

As used herein, the term “sensitivity” is the percentage of biomarker-detected subjects with a particular disease.

As used herein, “sample” or “biological sample” refers to anything, which may contain the biomarker (e.g., polypeptide, polynucleotide, or fragment thereof) for which a biomarker assay is desired. The sample may be a biological sample, such as a biological fluid or a biological tissue. In one embodiment, a biological sample is a tissue sample including pulmonary vascular cells. Such a sample may include diverse cells, proteins, and genetic material. Examples of biological tissues also include organs, tumors, lymph nodes, arteries and individual cell(s). Examples of biological fluids include urine, blood, plasma, serum, saliva, semen, stool, sputum, cerebral spinal fluid, tears, mucus, amniotic fluid or the like.

As used herein, the term “sensitivity” is the percentage of biomarker-detected subjects with a particular disease.

The terms “short RNA profile” or “RNA profile” or “tRNA fragment profile” are used interchangeably and refer to a genetic makeup of the RNA molecules that are present in a sample, such as a cell, tissue, or subject. Optionally, the abundance of an RNA molecule that is part of an RNA profile may also be sought. Optionally, other attributes of an RNA molecule that is part of an RNA profile may also be sought and include but are not limited to a molecule's location within the genomic locus of origin, the molecule's starting point, the molecule's ending point, the molecule's length, the identity of the molecule's terminal modifications, etc. The RNA molecules that can be used to form such a profile can be miRNAs, mRNAs, tRNAs, tRNA fragments, etc. as well as combinations thereof.

The term “signature” or “RNA signature” as used herein refers to a subset of an RNA profile and comprises the identity of one or more molecules that are selected from an RNA profile and optionally one or more of the attributes of the one or more molecules that are selected from the RNA profile.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%/a, more preferably 80% or 85%, and more preferably 90%, 95% or even 99% identical at the amino acid level or nucleic acid to the sequence used for comparison.

A “suppressor tRNA” refers to a tRNA that alters the reading of a messenger RNA (mRNA) in a given translation system, e.g., by providing a mechanism for incorporating an amino acid into a polypeptide chain in response to a selector codon. For example, a suppressor tRNA can read through, e.g., a stop codon, a four base codon, a rare codon, and/or the like

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disease or condition being treated. The therapeutically effective amount will vary depending on the compound, the disease and its severity and the age, weight, etc., of the subject to be treated.

The term “therapeutic” as used herein means a treatment and/or prophylaxis. A therapeutic effect is obtained by suppression, remission, or eradication of a disease state.

As used herein, the terms “treat,” treating,” “treatment,” and the like refer to reducing or improving a disease or condition and/or symptom associated therewith. It will be appreciated that, although not precluded, treating a disease or condition does not require that the disease, condition or symptoms associated therewith be completely ameliorated or eliminated.

The terms “tRNA fragment” or “tRF” (occasionally also referred to by us as “kuroko-RNA” or “kRNA”) are all used to refer to functional short non-coding RNAs generated from a tRNA locus. tRNA fragments have lengths that range from 10 to 40 or more nucleotides. Five structural categories of tRNA fragments include, the 5′-tRFs, the i-tRFs, the 3′-tRFs, the 5′-halves and the 3′-halves. The term “tRNA locus” refers to the genomic region that includes a tRNA gene and gives rise to the tRNA transcript. A given tRNA locus can produce zero, one, or more molecules belonging to zero, one, or more of the five structural categories.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### Description

The present invention includes methods and compositions of analyzing tRNA fragments. tRNAs are ancient non-coding RNAs (ncRNAs) that have been heretofore understood to be molecules with well-defined roles confined to the translation of messenger RNA (mRNA) into amino acid sequences. As such, tRNAs are present in archaea, bacteria, and eukaryotes. The conventional understanding had been that a genomic tRNA locus produces a single transcript that is processed to give rise to the mature tRNA. Described herein, tRNA loci also produce fragments that are important novel regulators with roles in regulation, cellular physiology, post-transcriptional regulation, etc. The specifics of how tRNAs and tRFs effect these roles are currently understood poorly. The present invention utilizes tRNA fragment profiling to identify subjects in need of therapeutic intervention.

In one aspect, a method of identifying a subject in need of therapeutic intervention to treat a disease or disease progression comprising, isolating fragments of tRNAs from a cell obtained from the subject; characterizing the fragments of tRNA and their relative abundance in the cell to identify a signature, wherein when the signature is indicative of a diagnosis of the disease or of a disease subtype treatment of the subject is recommended.

In another aspect, a method of identifying a cell's tissue of origin to treat a disease or disease progression or disease recurrence in a subject in need thereof comprising, isolating fragments of tRNAs from a cell obtained from the subject characterizing the fragments of tRNA and their relative abundance in the cell to identify a signature, wherein the signature is indicative of the cell's tissue of origin, or the disease status of the tissue of origin; and providing a treatment regimen to the subject dependent on the cell's tissue of origin, or the disease status of the tissue of origin.

tRNA Fragments

Analysis of tRNA fragment profiles or signatures in one or more cells can lead to the discovery of tRNA signatures present in healthy cells or diseased cells. tRNA signatures of one or more cells, or a tissue may be used to identify a diseased cell, disease progression, or disease recurrence in a subject. Thus, the subject may be identified as in need of therapeutic intervention to delay the onset of, reduce, improve, and/or treat a disease or condition, such as breast cancer, in a subject in need thereof.

Also provided is a panel of engineered oligonucleotides comprising a mixture of oligonucleotides that are about 5 to about 15 nucleotides (nts) in length and capable of hybridizing tRNA fragments and/or tRNAs, wherein the tRNA fragments are generally at least 15 nts in length and the tRNAs are generally less than 80 nts in length. The panel may include one or more oligonucleotides that may be used to identify tRNA fragments or tRNAs through hybridization of complementary regions between the oligonucleotides and the tRNAs, or related techniques that are well known to those skilled in the art. The oligonucleotides may include complementary sequences to known tRNA sequences, such as tRNA fragments. The oligonucleotides may be engineered to be between about 5 nucleotides to about 40 nucleotides, or about 5 nucleotides to about 30 nucleotides, or about 5 nucleotides to about 20 nucleotides, or about 5 nucleotides to about 15 nucleotides in length. The panel may include engineered oligonucleotides that are specific to a cell type, disease type, disease subtype, stage of disease, a patient's gender, a patient's population of origin, a patient's race or other aspect that may differentiate tRNA fragment signatures. The kits and oligonucleotide panel may also be used to identify agents that modulate disease, or progression of disease, or disease recurrence, in patient samples, and/or in in vitro or in vivo animal models for the disease at hand.

In another aspect, the invention includes a method for identifying tRNA fragments from sequenced reads, typically obtained through next generation sequencing approaches. The method comprises the steps of defining tRNA loci; mapping the sequenced reads to at least one tRNA genomic locus comprising disregarding map locations that differ from the tRNA fragments by at least an insertion, deletion, or replacement of a nucleotide, excluding tRNA fragments that map to locations outside of the tRNA loci and disregarding sequenced reads with tRNA intron sequences; mapping sequenced reads that are post-transcriptionally modified; and characterizing the remaining sequenced reads.

Known tRNA loci include the mitochondrial genome loci of mitochondrial tRNA sequences, the nuclear genome loci of nuclear tRNA sequences, and the nuclear genome loci of some mitochondrial tRNA sequences. Currently, there are the 22 known human mitochondrial tRNA sequences in the mitochondrial genome. There are 610 (508 true tRNAs and 102 pseudo-tRNAs) nuclear tRNA sequences in the nuclear genome, as per the public genomic tRNA database “GtRNAdb.” Selenocysteine tRNAs, tRNAs with undetermined anticodon identity, and tRNAs mapping to contigs that were not part of the human chromosome assembly are excluded from the collection of tRNA sequences considered here. Including the selenocysteine tRNAs, tRNAs with undetermined anticodon identity, and tRNAs mapping to contigs not part of the human chromosome assembly would render 625 nuclear tRNA sequences. There are also eight intervals in the nuclear genome, chr1:+:566062-566129, chr1:+:568843-568912, chr1:−:564879-564950, chr1:−:566137-566205, chr14:+:32954252-32954320, chr1:−:566207-566279, chr1:−: 567997-568065, and, chr5:−:93905172-93905240, that correspond to identical instances of seven mitochondrial tRNAs TrpTCA, LysTTT, GlnTTG, AlaTGC (×2), AsnGTT, SerTGA, and, GluTTC, respectively.

The sequenced reads are further mapped to at least one tRNA genomic locus. Sequenced reads that differ from the map location by at least an insertion, deletion, or replacement of a nucleotide are disregarded. Thusly, for examples, two distinct 5′-tRF molecules that would otherwise be indistinguishable can then be differentiated from one another and properly mapped. Also, the misidentification of the genomic origin of a sequenced read that would lead to erroneous results can be avoided.

The human genome is also riddled with many nuclear and mitochondrial tRNA-lookalikes, as well as partial tRNA sequences. Excluding sequenced reads that map to locations outside of the tRNA loci prevents the tRNA-like fragments from being included and considered further.

Also disregarding sequenced reads with tRNA intron sequences improves identification of bonafide tRNA fragments. Many tRNAs include intronic sequences. Sequenced reads that include only exonic sequences of an intron-containing tRNA are included. Sequenced reads that straddle a tRNA's exon-exon junction are further examined for possible mapping outside tRNA loci: any such reads that map outside tRNA loci are discarded to avoid erroneous results.

tRNA fragments are also prone to post-transcriptional modifications. Mature tRNAs are commonly modified with a CCA trinucleotide added to the 3′ end. Without explicit provisions to include these tRNA fragments, they would be inadvertently excluded from consideration by lacking an exact genomic map location. However, simply allowing an adequate number of mismatches (e.g. replacements) during mapping the nontemplated CCA is not adequate. Prior to mapping, a modification to the genome is created where the trinucleotide CCA is used to replace the three genomic nucleotides immediately downstream of each of the reference mature tRNAs. Special care must be taken. Otherwise, a careless replacement of the genomic sequence downstream from a tRNA by the CCA trinucleotide could inadvertently “erase” part of an adjacent tRNA's sequence as is the case, for example, for some tRNAs in the mitochondrial genome.

The tRNA fragments thusly identified are characterized. The tRNA fragments can be assessed for one or more of, sequence of the tRNA fragments, the overall abundance of the tRNA fragments based on the number of sequenced reads that mapped to tRNA loci, the relative abundance of a tRNA fragment to a reference, the length of a tRNA fragment, the starting and ending points of a tRNA fragment, the genomic origin of a tRNA fragment, the terminal modifications of a tRNA fragment, and other analyses known in the art.

In another aspect, a system is described herein to perform the method of identifying tRNA fragments. In one embodiment, the system comprises a processor that aligns sequenced reads with a genome and processes the alignment. The processor of the system processes the alignments and disregards data from the alignments when the mapped sequenced reads differ from the genome by at least an insertion, deletion, or replacement of a nucleotide; the mapped sequenced reads align to locations in the genome that reside outside of designated tRNA loci; the sequenced reads map to locations in the genome that reside both inside and outside of designated tRNA loci; or the mapped sequenced reads span intron sequences of tRNAs. The portion of the algorithm that is run by the processor of the system and which processes the alignments may also have provisions to include sequenced reads that correspond to post-transcriptionally modified molecules and would otherwise not align perfectly with the genome.

### Diagnostics

Samples from subjects suffering from a disease or a condition have a specific tRNA fragment profile in the cell or cells that are diseased, including metastastic cancer cells. Identifying the cellular origin or tissue origin of a cancer metastasis, or a propensity for a cell to metasize by identifying a tRNA fragment profile associated with the cellular origin or tissue origin or a propensity to metasize in a sample obtained from the subject allows the subject to undergo a recommended treatment. In one aspect, the invention includes a method of identifying a cell's tissue of origin to treat a disease or disease progression, or disease recurrence in a subject in need thereof comprising isolating fragments of tRNAs from a cell obtained from the subject; characterizing the fragments of tRNA, which can include assessing one or more of, overall abundance, relative abundance, length of the fragment, starting and ending points of the fragment, terminal modifications, etc., in the cell to identify a signature, wherein the signature is indicative of the cell's tissue of origin, and/or disease status of the tissue of origin; and providing a treatment regimen to the subject dependent on the cell's tissue of origin and/or disease status of the tissue of origin.

In another embodiment, characterizing the tRNA fragments that are present in the RNA profile can identify subjects in need of treatment.

In one embodiment, analyzing the length of tRNA fragments in a cell, tissue or body fluid is used to identify subjects in need of treatment. In a particular embodiment, a subset of tRNA fragments in breast tissue are analyzed as having a length of 19 nt or 20 nt. A predominance of 19 nt tRNA fragments in breast tissue is indicative of a breast tumor. In contrast, a predominance of 20 nt tRNA fragments in breast tissue is indicative of healthy breast tissue.

In another embodiment, analyzing the length of tRNA fragments in cell, tissue or body fluid is used to identify subjects with a disease subtype. In a particular embodiment, a subset of tRNA fragments in breast tumor tissue are analyzed as having a length of 16 nt, 17 nt, 26 nt, or 29 nt. A predominance of 16 nt and 17 nt tRNA fragments in breast tumor tissue is indicative of a triple negative breast cancer. A predominance of 17 nt and 29 nt tRNA fragments in breast tumor tissue is indicative of ER-positive breast cancer. A predominance of 26 nt tRNA fragments in breast tumor tissue is indicative of HER2-positive breast cancer.

In yet another embodiment, the relative abundance of the tRNA fragments that are present in the RNA profile can identify subjects in need of treatment. In another approach, diagnostic methods are used to assess tRNA fragment profiles in a biological sample relative to a reference (e.g., tRNA fragment profile in a healthy cell or tissue or body fluid in a corresponding control sample). Examples of a body fluid may include, but are not limited to, amniotic fluid, aqueous humour and vitreous humour, bile, blood serum, breast milk, cerebrospinal fluid, cerumen, chyle, chyme, endolymph and perilymph, exudates, feces, female ejaculate, gastric acid, gastric juice, lymph, mucus, pericardial fluid, peritoneal fluid, pleural fluid, pus, rheum, saliva, sebum, serous fluid, semen, smegma, sputum, synovial fluid, sweat, tears, urine, vaginal secretion, and vomit.

In one embodiment, the sample, such as a cell or tissue or body fluid is obtained from the subject. In another embodiment, the cell or tissue or body fluid is isolated from the sample. In another embodiment, the cell or tissue is isolated from a body fluid. The sample may be a peripheral blood cell, a tumor cell, a circulating tumor cell, an exosome, a bone marrow cell, a breast cell, a lung cell, a pancreatic cell, or other cell of the body.

In another embodiment, a signature of tRNA fragments or a presence or absence of specific tRNA fragments are indicative a diagnosis of a disease or condition. In a particular embodiment, the methods or assays described herein can comprise analyzing the presence or absence or the signature of tRNA fragments to analyze brain can include, without limitations, at least one of the sequences with identifiers SEQ ID NOs: 1-1802.

In another embodiment, the methods or assays described herein can comprise analyzing the presence or absence or the signature of tRNA fragments to analyze breast tissue can include, without limitations, at least one of the sequences with identifiers SEQ ID NOs: 8538-8852.

In yet another embodiment, the methods or assays described herein can comprise analyzing the presence or absence or the signature of tRNA fragments to analyze blood cells can include, without limitations, at least one of the sequences with identifiers SEQ ID NOs: 12462-14475.

In still another embodiment, the methods or assays described herein can comprise analyzing the presence or absence or the signature of tRNA fragments to analyze blood cells can include, without limitations, at least one of the sequences with identifiers SEQ ID NOs: 24833-25945.

In another embodiment, the methods or assays described herein can comprise analyzing the presence or absence or the signature of tRNA fragments to analyze pancreatic tissue can include, without limitations, at least one of the sequences with identifiers SEQ ID NOs: 36100-37466.

In another embodiment, the methods or assays described herein can comprise analyzing the presence or absence or the signature of tRNA fragments to analyze prostate tissue can include, without limitations, at least one of the sequences with identifiers SEQ ID NOs: 42349-43721.

In another embodiment, the methods or assays described herein can comprise analyzing the presence or absence or the signature of tRNA fragments to analyze platelets can include, without limitations, at least one of the sequences with identifiers SEQ ID NOs: 51286-51793.

In one embodiment, the methods or assays described herein can comprise detecting the presence or absence of one or more tRNA fragments to distinguish Alzheimer's disease brain from normal brain can include, without limitations, at least one of the sequences with identifiers SEQ ID NOs: 11, 18, 19, 28, 31, 34, 43, 51, 59, 83, 189, 194, 209, 268, 305, 306, 307, 316, 320, 398, 404, 611, 632, 653, 696, 751, 768, 816, 817, 860, 869, 870, 871, 920, 921, 925, 951, 960, 967, 989, 1005, 1030, 1133, 1201, 1202, 1223, 1229, 1230, 1231, 1240, 1248, 1298, 1318, 1406, 1412, 1421, 1425, 1453, 1510, 1577, 1582, 1631, 1637, 1645, 1661, 1695, 1727, 1794, or any combinations comprising two or more of these sequences.

In another embodiment, the methods or assays described herein can comprise detecting the presence or absence of one or more tRNA fragments to distinguish triple negative breast cancer from HER2+ breast cancer can include, without limitations, at least one of the sequences with identifiers SEQ ID NO:8613 or SEQ ID NO: 8823 or a combination comprising these sequences.

In yet another embodiment, the methods or assays described herein can comprise detecting the presence or absence of one or more tRNA fragments to distinguish triple negative breast cancer from normal can include, without limitations, at least one of the sequences with identifiers SEQ ID NOs: 8542, 8543, 8566, 8579, 8582, 8587, 8589, 8590, 8594, 8671-8673, 8707, 8731, 8774-8778, 8803, 8827-8828, 8831-8832, 8837-8838, 8852, or any combinations comprising two or more of these sequences.

In still another embodiment, the methods or assays described herein can comprise detecting the presence or absence of one or more tRNA fragments distinguish triple positive breast cancer from normal can include, without limitations, at least one of the sequences with identifiers SEQ ID NOs: 8540, 8566, 8575, 8579, 8589-8590, 8593-8594, 8775-8776, 8803, 8827-8828, 8837-8838, 8852, or any combinations comprising two or more of these sequences.

In another embodiment, the methods or assays described herein can comprise detecting the presence or absence of one or more tRNA fragments to distinguish triple positive breast cancer from triple negative breast cancer can include, without limitations, at least one of the sequences with identifiers SEQ ID NOs: 8596, 8601, 8622, 8657, 8664, 8811, or any combinations comprising two or more of these sequences.

In yet another embodiment, the methods or assays described herein can comprise detecting the presence or absence of one or more tRNA fragments to distinguish breast cancer from normal tissue can include, without limitations, at least one of the sequences with identifiers SEQ ID NOs: 8582, 8599-8601, 8622-8623, 8634, 8657, 8663-8665, 8676, 8698, 8703-8706, 8718-8720, 8722, 8724, 8738, 8745, 8758, 8761, 8767-8772, 8840, or any combinations comprising two or more of these sequences.

In still another embodiment, the methods or assays described herein can comprise detecting the presence or absence of one or more tRNA fragments to distinguish chronic lymphocytic leukemia from normal B-cells can include, without limitations, at least one of the sequences with identifiers SEQ ID NOs: 12462, 12463, 12464, 12465, 12466, 12467, 12468, 12469, 12470, 12471, 12472, 12473, 12474, 12475, 12476, 12477, 12478, 12479, 12480, 12481, 12482, 12483, 12484, 12485, 12486, 12487, 12488, 12489, 12490, 12492, 12493, 12494, 12495, 12496, 12497, 12498, 12499, 12500, 12501, 12502, 12503, 12504, 12505, 12506, 12507, 12508, 12509, 12510, 12511, 12512, 12513, 12514, 12515, 12516, 12517, 12518, 12519, 12520, 12522, 12523, 12524, 12525, 12526, 12527, 12529, 12530, 12531, 12532, 12533, 12534, 12536, 12537, 12538, 12540, 12541, 12542, 12543, 12544, 12545, 12546, 12547, 12548, 12549, 12550, 12551, 12552, 12553, 12554, 12555, 12556, 12557, 12558, 12559, 12560, 12561, 12562, 12563, 12564, 12565, 12566, 12567, 12568, 12569, 12570, 12572, 12573, 12574, 12575, 12576, 12577, 12578, 12580, 12581, 12582, 12584, 12585, 12586, 12587, 12588, 12589, 12590, 12591, 12592, 12593, 12594, 12595, 12596, 12597, 12598, 12599, 12600, 12601, 12602, 12603, 12604, 12607, 12608, 12609, 12614, 12615, 12616, 12617, 12618, 12619, 12620, 12621, 12622, 12623, 12624, 12625, 12626, 12627, 12628, 12629, 12631, 12632, 12633, 12634, 12635, 12636, 12637, 12638, 12639, 12640, 12641, 12642, 12643, 12645, 12647, 12648, 12649, 12652, 12653, 12654, 12655, 12657, 12658, 12659, 12660, 12661, 12663, 12664, 12665, 12666, 12667, 12668, 12669, 12670, 12671, 12672, 12674, 12677, 12678, 12679, 12680, 12682, 12684, 12685, 12686, 12687, 12688, 12689, 12690, 12691, 12692, 12693, 12694, 12695, 12696, 12697, 12698, 12699, 12700, 12703, 12704, 12705, 12706, 12708, 12710, 12711, 12712, 12713, 12714, 12715, 12716, 12717, 12718, 12719, 12720, 12721, 12724, 12726, 12727, 12728, 12729, 12730, 12731, 12732, 12733, 12734, 12736, 12738, 12739, 12740, 12741, 12742, 12743, 12744, 12745, 12746, 12747, 12749, 12750, 12751, 12754, 12756, 12758, 12760, 12761, 12763, 12764, 12765, 12766, 12767, 12768, 12769, 12770, 12771, 12773, 12774, 12776, 12777, 12779, 12780, 12781, 12782, 12783, 12785, 12786, 12788, 12789, 12790, 12791, 12792, 12795, 12799, 12800, 12801, 12802, 12803, 12804, 12805, 12806, 12807, 12809, 12811, 12812, 12813, 12814, 12815, 12817, 12818, 12819, 12820, 12821, 12824, 12825, 12826, 12827, 12828, 12829, 12831, 12832, 12833, 12834, 12835, 12836, 12837, 12838, 12840, 12841, 12842, 12843, 12844, 12846, 12847, 12848, 12849, 12850, 12851, 12852, 12853, 12854, 12855, 12856, 12857, 12858, 12859, 12860, 12861, 12864, 12865, 12867, 12868, 12869, 12870, 12871, 12872, 12873, 12874, 12875, 12876, 12877, 12878, 12879, 12880, 12881, 12882, 12883, 12884, 12885, 12886, 12887, 12888, 12889, 12890, 12891, 12892, 12893, 12894, 12895, 12896, 12897, 12899, 12900, 12901, 12902, 12903, 12904, 12905, 12906, 12907, 12909, 12910, 12911, 12912, 12913, 12914, 12916, 12918, 12919, 12920, 12922, 12923, 12924, 12925, 12926, 12927, 12928, 12929, 12930, 12931, 12932, 12933, 12934, 12935, 12936, 12937, 12938, 12939, 12940, 12941, 12942, 12943, 12944, 12946, 12947, 12948, 12949, 12950, 12951, 12954, 12955, 12956, 12957, 12958, 12959, 12960, 12961, 12962, 12963, 12965, 12966, 12967, 12968, 12969, 12970, 12971, 12972, 12973, 12974, 12975, 12978, 12979, 12980, 12981, 12982, 12983, 12984, 12985, 12986, 12987, 12988, 12990, 12991, 12992, 12993, 12994, 12996, 12997, 12998, 12999, 13000, 13001, 13002, 13003, 13004, 13005, 13006, 13007, 13008, 13009, 13011, 13012, 13013, 13014, 13016, 13017, 13018, 13019, 13020, 13021, 13022, 13023, 13024, 13025, 13028, 13029, 13030, 13031, 13033, 13034, 13035, 13036, 13037, 13038, 13039, 13040, 13044, 13045, 13046, 13047, 13049, 13050, 13051, 13052, 13053, 13054, 13055, 13056, 13057, 13058, 13059, 13061, 13063, 13065, 13066, 13067, 13068, 13069, 13070, 13071, 13072, 13073, 13074, 13075, 13076, 13077, 13078, 13079, 13080, 13081, 13082, 13083, 13084, 13085, 13086, 13087, 13088, 13089, 13090, 13091, 13092, 13093, 13094, 13095, 13096, 13097, 13098, 13100, 13101, 13102, 13103, 13104, 13105, 13106, 13107, 13110, 13112, 13113, 13114, 13117, 13118, 13119, 13120, 13121, 13122, 13123, 13124, 13125, 13127, 13128, 13129, 13130, 13131, 13132, 13133, 13134, 13135, 13136, 13137, 13138, 13139, 13140, 13141, 13142, 13143, 13145, 13146, 13148, 13149, 13150, 13151, 13152, 13153, 13154, 13155, 13157, 13158, 13159, 13160, 13161, 13162, 13163, 13164, 13165, 13166, 13167, 13168, 13169, 13170, 13171, 13174, 13175, 13177, 13178, 13179, 13181, 13182, 13183, 13184, 13185, 13186, 13187, 13189, 13190, 13191, 13193, 13195, 13196, 13198, 13199, 13200, 13201, 13202, 13203, 13204, 13205, 13206, 13207, 13208, 13209, 13210, 13211, 13212, 13213, 13214, 13215, 13216, 13217, 13218, 13219, 13221, 13222, 13223, 13225, 13228, 13230, 13231, 13232, 13233, 13234, 13236, 13237, 13238, 13239, 13240, 13241, 13242, 13243, 13245, 13246, 13247, 13248, 13249, 13250, 13251, 13252, 13253, 13255, 13256, 13257, 13258, 13259, 13260, 13261, 13262, 13263, 13264, 13268, 13269, 13270, 13271, 13273, 13274, 13275, 13276, 13277, 13278, 13279, 13280, 13281, 13283, 13285, 13286, 13287, 13288, 13289, 13290, 13292, 13293, 13294, 13295, 13296, 13297, 13298, 13299, 13300, 13301, 13302, 13303, 13304, 13306, 13309, 13310, 13312, 13313, 13314, 13315, 13316, 13317, 13318, 13319, 13320, 13323, 13324, 13325, 13326, 13327, 13328, 13329, 13330, 13331, 13332, 13333, 13334, 13335, 13336, 13337, 13338, 13339, 13340, 13341, 13342, 13343, 13345, 13346, 13347, 13348, 13349, 13350, 13351, 13352, 13353, 13354, 13355, 13357, 13358, 13359, 13360, 13361, 13362, 13363, 13364, 13365, 13366, 13367, 13369, 13370, 13371, 13372, 13373, 13374, 13375, 13376, 13377, 13378, 13379, 13380, 13381, 13382, 13383, 13384, 13385, 13386, 13387, 13388, 13389, 13390, 13391, 13392, 13393, 13394, 13395, 13396, 13397, 13398, 13399, 13400, 13401, 13402, 13403, 13404, 13405, 13406, 13407, 13408, 13409, 13410, 13411, 13412, 13413, 13414, 13415, 13416, 13417, 13421, 13422, 13424, 13426, 13427, 13428, 13429, 13430, 13431, 13432, 13433, 13434, 13436, 13437, 13438, 13439, 13440, 13441, 13442, 13443, 13445, 13446, 13447, 13448, 13449, 13450, 13452, 13453, 13454, 13455, 13456, 13457, 13458, 13459, 13460, 13461, 13462, 13463, 13464, 13465, 13466, 13467, 13468, 13469, 13470, 13471, 13472, 13473, 13474, 13475, 13476, 13477, 13478, 13479, 13480, 13481, 13482, 13484, 13485, 13486, 13488, 13489, 13491, 13492, 13493, 13494, 13495, 13496, 13498, 13500, 13501, 13503, 13504, 13505, 13506, 13507, 13508, 13509, 13510, 13511, 13512, 13513, 13514, 13516, 13517, 13519, 13520, 13522, 13523, 13524, 13525, 13528, 13529, 13530, 13531, 13532, 13533, 13534, 13535, 13536, 13537, 13538, 13539, 13540, 13541, 13542, 13543, 13544, 13545, 13546, 13547, 13548, 13550, 13551, 13552, 13553, 13554, 13556, 13557, 13558, 13559, 13560, 13561, 13562, 13563, 13567, 13568, 13569, 13570, 13571, 13572, 13573, 13574, 13576, 13577, 13578, 13579, 13580, 13581, 13582, 13583, 13584, 13585, 13586, 13587, 13588, 13589, 13590, 13591, 13592, 13593, 13594, 13595, 13596, 13597, 13598, 13599, 13600, 13601, 13602, 13603, 13604, 13605, 13606, 13607, 13608, 13609, 13610, 13611, 13612, 13613, 13614, 13615, 13616, 13617, 13619, 13620, 13621, 13622, 13623, 13624, 13626, 13627, 13628, 13629, 13632, 13633, 13634, 13635, 13636, 13637, 13638, 13639, 13640, 13641, 13642, 13643, 13644, 13645, 13646, 13647, 13648, 13649, 13650, 13651, 13654, 13655, 13656, 13657, 13658, 13659, 13660, 13661, 13662, 13663, 13664, 13665, 13666, 13667, 13668, 13669, 13670, 13671, 13672, 13673, 13674, 13675, 13676, 13677, 13678, 13679, 13680, 13681, 13682, 13683, 13684, 13685, 13687, 13688, 13690, 13691, 13693, 13695, 13696, 13697, 13699, 13700, 13702, 13703, 13704, 13706, 13707, 13708, 13709, 13710, 13711, 13712, 13713, 13714, 13716, 13717, 13718, 13719, 13720, 13721, 13722, 13723, 13724, 13725, 13726, 13727, 13728, 13729, 13730, 13731, 13732, 13733, 13734, 13735, 13737, 13738, 13739, 13740, 13741, 13742, 13743, 13744, 13745, 13746, 13747, 13748, 13749, 13750, 13751, 13752, 13754, 13755, 13756, 13757, 13758, 13759, 13760, 13762, 13763, 13764, 13765, 13766, 13767, 13768, 13769, 13770, 13771, 13772, 13774, 13775, 13776, 13777, 13778, 13779, 13780, 13781, 13782, 13783, 13784, 13785, 13786, 13787, 13788, 13789, 13790, 13792, 13793, 13794, 13795, 13796, 13799, 13801, 13802, 13803, 13804, 13806, 13807, 13808, 13809, 13810, 13811, 13812, 13813, 13815, 13816, 13817, 13818, 13819, 13820, 13821, 13822, 13823, 13824, 13825, 13826, 13827, 13828, 13829, 13830, 13831, 13833, 13834, 13835, 13836, 13837, 13838, 13839, 13841, 13842, 13843, 13844, 13845, 13846, 13849, 13850, 13851, 13852, 13853, 13854, 13855, 13856, 13857, 13858, 13859, 13860, 13861, 13862, 13863, 13864, 13865, 13866, 13868, 13869, 13870, 13871, 13873, 13874, 13875, 13876, 13878, 13879, 13880, 13881, 13882, 13884, 13885, 13887, 13888, 13889, 13890, 13893, 13895, 13896, 13897, 13898, 13899, 13900, 13901, 13902, 13903, 13904, 13905, 13906, 13908, 13909, 13910, 13911, 13912, 13914, 13915, 13916, 13917, 13919, 13920, 13921, 13922, 13923, 13924, 13925, 13926, 13928, 13929, 13930, 13931, 13932, 13933, 13934, 13935, 13936, 13937, 13938, 13939, 13940, 13941, 13942, 13944, 13945, 13946, 13948, 13950, 13952, 13953, 13954, 13955, 13956, 13960, 13961, 13962, 13963, 13964, 13965, 13966, 13967, 13968, 13970, 13971, 13972, 13973, 13974, 13975, 13976, 13977, 13978, 13979, 13980, 13982, 13983, 13984, 13985, 13986, 13987, 13988, 13989, 13990, 13991, 13992, 13993, 13994, 13995, 13996, 13997, 13998, 13999, 14000, 14001, 14002, 14003, 14004, 14005, 14006, 14007, 14008, 14010, 14011, 14012, 14013, 14014, 14015, 14016, 14017, 14018, 14019, 14020, 14021, 14022, 14023, 14024, 14025, 14026, 14027, 14028, 14030, 14031, 14032, 14034, 14035, 14037, 14038, 14039, 14040, 14041, 14042, 14043, 14044, 14045, 14046, 14047, 14048, 14049, 14050, 14051, 14052, 14053, 14055, 14059, 14060, 14061, 14062, 14064, 14065, 14067, 14068, 14069, 14070, 14071, 14072, 14073, 14074, 14075, 14076, 14077, 14078, 14079, 14080, 14082, 14084, 14085, 14086, 14088, 14089, 14090, 14092, 14093, 14095, 14096, 14097, 14098, 14099, 14100, 14103, 14104, 14105, 14108, 14109, 14110, 14111, 14112, 14113, 14116, 14117, 14118, 14119, 14121, 14122, 14123, 14124, 14125, 14126, 14127, 14128, 14129, 14130, 14131, 14132, 14133, 14135, 14136, 14137, 14139, 14141, 14142, 14143, 14144, 14145, 14146, 14147, 14148, 14151, 14152, 14153, 14154, 14155, 14156, 14157, 14158, 14159, 14160, 14161, 14162, 14163, 14166, 14167, 14168, 14169, 14170, 14171, 14172, 14173, 14175, 14176, 14177, 14178, 14179, 14180, 14181, 14182, 14183, 14185, 14186, 14187, 14188, 14190, 14191, 14192, 14193, 14194, 14195, 14197, 14198, 14199, 14201, 14204, 14205, 14207, 14208, 14212, 14213, 14215, 14216, 14217, 14218, 14219, 14222, 14223, 14224, 14225, 14226, 14227, 14228, 14229, 14230, 14231, 14232, 14233, 14234, 14235, 14236, 14237, 14238, 14239, 14240, 14241, 14242, 14243, 14244, 14245, 14246, 14247, 14248, 14249, 14250, 14251, 14252, 14253, 14254, 14255, 14256, 14257, 14258, 14259, 14260, 14261, 14262, 14263, 14265, 14266, 14267, 14268, 14271, 14273, 14274, 14276, 14280, 14281, 14282, 14283, 14284, 14285, 14287, 14288, 14290, 14292, 14293, 14294, 14295, 14296, 14297, 14298, 14299, 14300, 14301, 14302, 14303, 14304, 14305, 14306, 14307, 14308, 14309, 14310, 14311, 14313, 14314, 14315, 14316, 14317, 14320, 14321, 14322, 14323, 14324, 14325, 14326, 14328, 14329, 14330, 14331, 14332, 14333, 14334, 14335, 14336, 14338, 14339, 14340, 14342, 14343, 14344, 14346, 14347, 14348, 14349, 14350, 14351, 14353, 14354, 14355, 14356, 14357, 14358, 14359, 14360, 14361, 14363, 14365, 14366, 14367, 14368, 14369, 14370, 14371, 14372, 14373, 14374, 14375, 14376, 14377, 14378, 14379, 14380, 14382, 14383, 14384, 14385, 14386, 14389, 14390, 14391, 14392, 14393, 14394, 14395, 14396, 14397, 14399, 14400, 14401, 14402, 14403, 14404, 14405, 14406, 14407, 14408, 14409, 14410, 14411, 14412, 14413, 14415, 14416, 14417, 14418, 14419, 14420, 14421, 14422, 14424, 14427, 14428, 14429, 14430, 14432, 14434, 14435, 14436, 14437, 14438, 14440, 14441, 14442, 14443, 14444, 14445, 14446, 14447, 14448, 14450, 14451, 14452, 14453, 14454, 14455, 14456, 14457, 14458, 14459, 14460, 14461, 14463, 14465, 14467, 14469, 14470, 14471, 14473, 14475, or any combinations comprising two or more of these sequences.

In another embodiment, the methods or assays described herein can comprise detecting the presence or absence of one or more tRNA fragments to distinguish B-cells from breast cells can include, without limitations, at least one of the sequences with identifiers SEQ ID NOs: 24995-24996, 25025, 25031, 25033, 25087-25091, 25093-25094, 25128, 25150, 25161-25162, 25165, 25182, 25219-25220, 25230, 25277-25278, 25284, 25316, 25356-25357, 25359-25360, 25363-25364, 25397-25398, 25415, 25424, 25432, 25480, 25484-25486, 25498-25499, 25505, 25524, 25550-25552, 25570, 25580, 25583, 25609-25610, 25619, 25646-25647, 25685-25687, 25691, 25714, 25720, 25727-25728, 25731, 25741, 25746-25747, 25846-25847, 25868, 25882, 25904, 25908-25912, 25914-25915, or any combinations comprising two or more of these sequences.

In yet another embodiment, the methods or assays described herein can comprise detecting the presence or absence of one or more tRNA fragments to distinguish B-cells from white people from B-cells from black people can include, without limitations, at least one of the sequences with identifiers SEQ ID NOs: 24880-24883, 24896-24897, 24959-24963, 24965, 24973, 25006, 25027, 25052, 25054, 25102-25103, 25110-25111, 25118, 25123, 25150, 25152-25153, 25183-25184, 25188, 25198, 25202, 25204-25206, 25210, 25212-25214, 25224-25225, 25245, 25252-25254, 25257, 25259-25261, 25270, 25273, 25286, 25294, 25296, 25313-25314, 25334, 25416, 25425, 25449-25450, 25454, 25476-25478, 25583, 25609-25612, 25665, 25667, 25705, 25714, 25786, 25894, 25896-25897, or any combinations comprising two or more of these sequences.

In still another embodiment, the methods or assays described herein can comprise detecting the presence or absence of one or more tRNA fragments to distinguish B-cells from men from B-cells from women can include, without limitations, at least one of the sequences with identifiers SEQ ID NO: 24881, 24926, 24952, 24981, 24990, 24995, 24998, 25010, 25047, 25051, 25075, 25101-25102, 2511 25111, 25118, 25121, 25149, 25211, 25218, 25238, 25309, 25359, 25373, 25376, 25386-25387, 25402, 25410, 25415-25416, 25420-25421, 25468, 25474, 25476, 25484-25487, 25493, 25524, 25536, 25560, 25596, 25604, 25620, 25631, 25651, 25662, 25664, 25714, 25723, 25803, 25829, 25850-25851, 25886-25887, 25898, 25902-25903, 25905, 25914, 25921, 25923, 25937, or any combinations comprising two or more of these sequences.

In another embodiment, the methods or assays described herein can comprise detecting the presence or absence of one or more tRNA fragments to distinguish normal pancreas from pancreatic cancer can include, without limitations, at least one of the sequences with identifiers SEQ ID NOs: 36100, 36101, 36105, 36107, 36111, 36112, 36114, 36115, 36116, 36119, 36120, 36121, 36122, 36123, 36139, 36143, 36146, 36147, 36148, 36149, 36155, 36156, 36157, 36163, 36171, 36173, 36176, 36177, 36178, 36179, 36180, 36181, 36182, 36183, 36188, 36189, 36194, 36197, 36200, 36203, 36204, 36215, 36217, 36218, 36219, 36222, 36223, 36227, 36228, 36230, 36231, 36234, 36238, 36239, 36240, 36241, 36242, 36243, 36246, 36248, 36252, 36254, 36262, 36265, 36266, 36269, 36270, 36271, 36272, 36273, 36276, 36278, 36279, 36282, 36285, 36287, 36288, 36289, 36293, 36294, 36295, 36296, 36297, 36298, 36299, 36303, 36304, 36305, 36306, 36307, 36308, 36313, 36319, 36320, 36322, 36323, 36326, 36327, 36331, 36332, 36333, 36335, 36336, 36338, 36339, 36341, 36342, 36344, 36347, 36355, 36356, 36357, 36372, 36373, 36374, 36375, 36376, 36378, 36381, 36384, 36387, 36391, 36392, 36395, 36397, 36399, 36400, 36401, 36405, 36406, 36408, 36409, 36428, 36429, 36430, 36431, 36432, 36433, 36435, 36436, 36437, 36444, 36450, 36451, 36452, 36453, 36455, 36456, 36457, 36460, 36461, 36462, 36463, 36464, 36465, 36466, 36467, 36468, 36469, 36470, 36471, 36472, 36478, 36485, 36490, 36491, 36498, 36499, 36504, 36505, 36506, 36507, 36508, 36509, 36510, 36511, 36512, 36513, 36517, 36520, 36521, 36523, 36524, 36529, 36530, 36533, 36534, 36535, 36538, 36539, 36541, 36542, 36543, 36544, 36545, 36546, 36547, 36550, 36553, 36554, 36561, 36562, 36572, 36573, 36574, 36575, 36578, 36579, 36580, 36581, 36582, 36584, 36586, 36589, 36590, 36591, 36593, 36594, 36597, 36599, 36600, 36601, 36607, 36608, 36609, 36610, 36611, 36612, 36614, 36615, 36616, 36617, 36618, 36619, 36620, 36621, 36627, 36628, 36629, 36637, 36638, 36639, 36640, 36641, 36642, 36643, 36644, 36645, 36646, 36647, 36649, 36650, 36658, 36665, 36669, 36670, 36671, 36673, 36674, 36675, 36676, 36677, 36678, 36679, 36680, 36682, 36683, 36684, 36689, 36690, 36691, 36692, 36693, 36694, 36695, 36696, 36697, 36698, 36701, 36702, 36703, 36705, 36706, 36707, 36708, 36709, 36710, 36711, 36712, 36714, 36715, 36716, 36718, 36719, 36720, 36721, 36722, 36726, 36727, 36728, 36729, 36730, 36731, 36732, 36733, 36734, 36735, 36738, 36739, 36741, 36742, 36744, 36745, 36746, 36747, 36749, 36751, 36754, 36755, 36756, 36757, 36759, 36760, 36761, 36762, 36763, 36764, 36765, 36768, 36769, 36770, 36771, 36772, 36775, 36776, 36777, 36778, 36788, 36789, 36793, 36794, 36796, 36797, 36798, 36799, 36800, 36803, 36805, 36806, 36809, 36810, 36812, 36814, 36817, 36825, 36826, 36827, 36829, 36830, 36831, 36832, 36834, 36835, 36838, 36839, 36841, 36844, 36846, 36848, 36849, 36851, 36854, 36855, 36857, 36859, 36860, 36861, 36862, 36863, 36864, 36868, 36869, 36871, 36872, 36877, 36878, 36879, 36880, 36881, 36883, 36884, 36885, 36886, 36887, 36889, 36890, 36891, 36892, 36895, 36897, 36901, 36902, 36903, 36904, 36905, 36907, 36909, 36910, 36911, 36913, 36914, 36915, 36916, 36917, 36918, 36919, 36925, 36931, 36938, 36939, 36941, 36942, 36945, 36946, 36948, 36952, 36953, 36955, 36956, 36957, 36958, 36961, 36963, 36964, 36965, 36967, 36968, 36973, 36976, 36977, 36978, 36979, 36980, 36981, 36982, 36983, 36985, 36988, 36989, 36990, 36991, 36992, 36997, 36998, 36999, 37001, 37004, 37005, 37008, 37009, 37012, 37013, 37014, 37021, 37022, 37023, 37024, 37025, 37026, 37029, 37032, 37033, 37036, 37039, 37044, 37046, 37048, 37049, 37050, 37051, 37054, 37055, 37056, 37057, 37058, 37059, 37060, 37063, 37065, 37066, 37075, 37077, 37078, 37079, 37080, 37081, 37083, 37087, 37088, 37089, 37090, 37091, 37094, 37095, 37099, 37100, 37101, 37110, 37115, 37116, 37117, 37119, 37120, 37121, 37123, 37124, 37125, 37127, 37132, 37133, 37134, 37135, 37137, 37138, 37139, 37141, 37142, 37143, 37144, 37145, 37146, 37149, 37150, 37151, 37152, 37155, 37157, 37160, 37161, 37162, 37163, 37164, 37165, 37166, 37167, 37168, 37169, 37171, 37174, 37175, 37177, 37178, 37181, 37182, 37183, 37184, 37185, 37187, 37193, 37194, 37195, 37196, 37197, 37198, 37199, 37201, 37202, 37203, 37206, 37207, 37208, 37209, 37211, 37213, 37214, 37216, 37217, 37226, 37227, 37228, 37229, 37230, 37231, 37234, 37235, 37237, 37244, 37245, 37247, 37248, 37249, 37251, 37253, 37254, 37255, 37261, 37262, 37265, 37271, 37272, 37273, 37274, 37278, 37279, 37283, 37303, 37304, 37305, 37306, 37307, 37308, 37312, 37316, 37319, 37321, 37323, 37324, 37325, 37326, 37327, 37334, 37335, 37336, 37337, 37338, 37339, 37340, 37341, 37342, 37348, 37356, 37363, 37365, 37368, 37369, 37370, 37372, 37374, 37375, 37376, 37382, 37383, 37385, 37386, 37388, 37391, 37394, 37395, 37398, 37400, 37401, 37402, 37403, 37404, 37405, 37407, 37408, 37410, 37419, 37420, 37422, 37423, 37424, 37425, 37426, 37429, 37430, 37431, 37432, 37433, 37445, 37446, 37448, 37449, 37453, 37454, 37456, 37461, 37462, 37463, 37464, 37466, or any combinations comprising two or more of these sequences.

In yet another embodiment, the methods or assays described herein can comprise detecting the presence or absence of one or more tRNA fragments to distinguish platelets from people with a propensity to clot vs. platelets from people with a propensity to hemorrhage can include, without limitations, at least one of the sequences with identifiers SEQ ID NOs: 51377-51378, 51406, 51438, 51496, 51565, 51691, 51699, 51736-51737, 51745, 51759, or any combinations comprising two or more of these sequences.

In still another embodiment, the methods or assays described herein can comprise detecting the presence or absence of one or more tRNA fragments to distinguish normal prostate from prostate cancer can include, without limitations, at least one of the sequences with identifiers SEQ ID NOs: 42434, 42520, 42537, 42577, 42751, 42979, 43019, 43090, 43128, 43156, 43310, 43352, 43398, 43426, 43437, or any combinations comprising two or more of these sequences.

In general, characterizing the tRNA fragments identifies a signature that may be indicative of a diagnosis of a disease or condition. The character of the tRNA fragments in the sample may be compared with a reference, such as other tRNAs present within the cell, a healthy cell or a diseased cell will yield a relative abundance of the tRNA fragments to identify a signature. The signature may be established by comparing the tRNA fragments' locations within the genomic loci of origin, the starting and ending points of the fragments, the length of the fragment, and any other feature of the fragments as compared to other tRNA fragments within the same sample or another sample or reference to distinguish a diseased state, a propensity to develop a disease or condition, and/or the absence of a disease or condition. The skilled artisan will appreciate that the diagnostic can be adjusted to increase sensitivity or specificity of the assay. In general, any significant increase (e.g., at least about 10%, 15%, 30%, 50%, 60%, 75%, 80%, or 90%) in the level of a polynucleotide or polypeptide biomarker in the subject sample relative to a reference may be used to diagnose a diseased state, a propensity to develop a disease or condition, and/or the absence of a disease or condition.

Accordingly, a tRNA fragment profile may be obtained from a sample from a subject and compared to a reference tRNA fragment profile obtained from a reference cell or tissue or body fluid, so that it is possible to classify the subject as belonging to or not belonging to the reference population. The correlation may take into account the presence or absence of one or more tRNA fragments in a test sample and the frequency of detection of the tRNA fragments in a test sample compared to a control. The correlation may take into account both of such factors to facilitate a diagnosis of a disease or condition. In one embodiment, the reference is the identity and abundance level of the tRNA fragments present in a control sample, such as non-diseased cell, a cell obtained from a patient that does not have the disease or condition at issue or a propensity to develop such a disease or condition. In another embodiment, the reference is a baseline level of the tRNA fragment presence and abundance in a biologic sample derived from the patient prior to, during, or after treatment for the disease or condition. In yet another embodiment, the reference is a standardized curve.

### Methods of Use

The method described herein includes diagnosing, identifying or monitoring a disease or condition, such as breast cancer, in a subject in need of therapeutic intervention. In one embodiment, the method includes isolating tRNA fragments from a cell, tissue or body fluid obtained from the subject; hybridizing the tRNA fragments to a panel of oligonucleotides engineered to detect tRNA fragments; analyzing an identity and levels of the tRNA fragments present in the cell; wherein a differential in the identity or measured tRNA fragments' levels to the reference is indicative of a diagnosis or identification of breast cancer in the subject; and providing a treatment regimen to the subject dependent on the differential in the identity and measured tRNA fragments' levels to the reference. The tRNAs may be isolated by a method known in the art or selected from the group consisting of size selection, sequencing, amplification, dumbbell-PCR and FIREPLEX®. In some embodiments, the size of the tRNA fragments is in the range of about 10 nucleotides to about 80 nucleotides are isolated. The range of sizes may include, but are not limited to, from about 15 nucleotides to about 55 nucleotides, and from about 17 nucleotides to about 52 nucleotides. The size of the tRNAs may be about 10 nucleotides, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 or about 80 nucleotides.

The signature is a tRNA fragment profile that comprises the identity, abundance and relative abundance of tRNA fragments. The tRNA fragments' location within the genomic loci of origin, the starting and ending points of the fragments, the length of the fragments, and any other feature of the fragments as compared to other tRNA fragments within the same sample or another sample or reference may be included in the tRNA fragment signature. In one embodiment, the signature is obtained by hybridization to a single oligonucleotide, or to a panel of oligonucleotides, such as those that comprise at least two or more oligonucleotides that selectively hybridize to the tRNAs. To prepare the sample for characterization, the tRNAs and tRNA fragments may be amplified prior to the hybridization.

The therapeutic methods (which include prophylactic treatments) to treat a disease or condition, such as a disease selected from the group consisting of a cancer, and genetically predisposed disease, in a subject include administering a therapeutically effective amount of an agent or therapeutic to a subject (e.g., animal, human) in need thereof, including a mammal, particularly a human. Such treatment will be suitably administered to subjects, particularly humans, suffering from, having, susceptible to, or at risk for the disease or condition or a symptom thereof. The agent may be identified in a screening using tRNA signatures or relative abundance of tRNAs in in vitro or in vivo animal model for the disease or condition.

### Monitoring

Methods of monitoring subjects that are at high risk of developing a disease or condition, or are at risk of disease or condition recurrence, or who are receiving therapeutic intervention to reduce, improve, or treat a symptom of the disease or condition, such as breast cancer, are also useful in determining whether to administer treatment and in managing treatment. Provided are methods where the tRNA fragments are measured and characterized. In some cases, the tRNA fragments are measured and characterized as part of a routine course of action. In other cases, the tRNA fragments are measured and characterized before and again after subject management or treatment. In these cases, the methods are used to monitor the onset of a disease or condition, the recurrence of the disease or condition, the status of the disease or condition, or a propensity to develop such disease or condition, e.g., breast cancer.

For example, characterization of tRNA fragments or signatures can be used to monitor a subject's response to certain treatments. Such characterization can be used to monitor for the presence or absence of the disease or condition. The changes in the relative abundance or tRNA signature delineated herein before treatment, during treatment, or following the conclusion of a treatment regimen may be indicative of the course of the disease or condition, progression of disease or condition, or response to treatment. In some embodiments, characterization of tRNA fragments or signatures may be assessed at one or more times (e.g., 2, 3, 4, 5). Analysis of the tRNA fragments are made, for example, using a size selection, sequencing, and amplification, or other standard method to determine the tRNA fragment profile. If desired, tRNA fragment profile is compared to a reference to determine if any alteration in the tRNA fragment profile is present. Such monitoring may be useful, for example, in assessing the efficacy of a particular treatment in a patient. Therapeutics that normalize the tRNA fragment profile are taken as particularly useful.

### Kits

Kits for diagnosing, identifying or monitoring a disease or condition, such as breast cancer, are included. In one aspect, the invention includes a panel of engineered oligonucleotides comprising a mixture of oligonucleotides that are about 5 to about 15 nucleotides (nts) in length and capable of hybridizing tRNAs and tRNA fragments, wherein the tRNAs and tRNA fragments are less than about 80 nts in length. In another aspect, the invention includes a kit for high-throughput analysis of tRNA or tRNA fragments in a sample comprising the panel of engineered oligonucleotides of claim 12; hybridization reagents; and tRNA isolation reagents. Other kits with variations on the components and olignucleotide panels may be used in the context of the present invention. For example, the panel of engineered oligonucleotides may be specific to a cell type, disease type, stage of disease, or other aspect that may differentiate tRNA signatures. The kits and oligonucleotide panel may also be used to identify agents that modulate disease, or progression of disease in in vitro or in vivo animal models for the disease.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, fourth edition (Sambrook, 2012); “Oligonucleotide Synthesis” (Gait, 1984); “Culture of Animal Cells” (Freshney, 2010); “Methods in Enzymology” “Handbook of Experimental Immunology” (Weir, 1997); “Gene Transfer Vectors for Mammalian Cells” (Miller and Calos, 1987); “Short Protocols in Molecular Biology” (Ausubel, 2002); “Polymerase Chain Reaction: Principles, Applications and Troubleshooting”, (Babar, 2011); “Current Protocols in Immunology” (Coligan, 2002). These techniques are applicable to the production of the polynucleotides and polypeptides of the invention, and, as such, may be considered in making and practicing the invention. Particularly useful techniques for particular embodiments will be discussed in the sections that follow.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The Results of the experiments disclosed herein are now described.

Transfer RNAs (tRNAs) are a class of non-coding RNAs (ncRNAs) and an integral component of the process of translating messenger RNAs (mRNAs) into the respective amino acid sequences. Each tRNA locus had been thought to give rise to a single transcript with a concrete single concrete role during the mRNA translation. However, recent studies have provided evidence that additional, functional short ncRNAs (“tRNA fragments” or “tRFs”) can also be generated from tRNA loci. TRNAs have traditionally been thought as producing a single transcript, the tRNA, a molecule that is an integral component of the process of translation of a messenger RNA (mRNA) into an amino acid sequence.

The present invention described herein includes two large datasets of short RNA profiles. Collections of tRNA fragments were generated and analyzed for several datasets. The results of the analyses were summarized in 7 tables on compact disc (CD) created on Oct. 27, 2014 that were submitted in the Provisional Patent Application No. 62/122,711, filed Oct. 28, 2014, where the results are in files: 205961-7006P2_Excel_Breast_TCGA.csv; 205961-7006P2_Excel_Brain_2N_2D.csv; 205961-7006P2_Excel_CLL.csv; 205961-7006P2_Excel_EBI_LCLs.csv; 205961-7006P2_Excel_Pancreas.csv; 205961-7006P2_Excel_Platelets.csv; and 205961-7006P2_Excel_Prostate.csv, all of which are incorporated by reference.

The two large public datasets of short RNA profiles that were systematically analyzed include: a first set that corresponds to lymphoblastoid cell lines derived from 452 individuals, men and women, from five different populations; and a second set that corresponds to 311 breast cancer samples from The Cancer Genome Atlas repository.

Nearly every known tRNA locus of the human genome gives rise to multiple and overlapping tRNA fragments with each fragment having concrete endpoints and a distinct length. Many of the discovered fragments are internal to the mature tRNA locus, i.e., they differ from previously reported 5′ and 3′ tRFs. The relative abundance and the endpoints of these tRNA fragments remain characteristically consistent across individuals, thus indicating a constitutive nature and a presumed participation in the molecular biology of the corresponding tissue through currently unknown interactions.

Importantly, the abundance and the choice of endpoints for these constitutive tRNA fragments depend on several features including tissue type, gender, population, ethnic background, and disease subtype. For a given locus, the choice of endpoints varied as a function of the amino acid and of the anticodon at hand. Independent experimental investigation of several previously unreported fragments found them to be present in model cell lines and in human tissues. Based on the findings, tRNA loci are rich sources of many distinct shorter tRNA fragments that can be functional molecules in addition to giving rise to mature tRNAs.

tRNAs are ancient non-coding RNAs (ncRNAs) that are present in archaea, bacteria, and eukaryotes. The role of tRNAs has been long presumed to be confined to the process of translation of a messenger RNA (mRNA) into an amino acid sequence. There is increasing evidence that tRNAs and tRNA fragments also have roles in cellular physiology, post-transcriptional regulation, etc. The specifics of how tRNAs and tRNA fragments effect these other events remains largely unclear.

The conventional understanding had been that genomic loci harboring tRNAs produce a single precursor transcript that is eventually processed and gives rise to the mature tRNA. Recent evidence, however, suggests that tRNA fragments, which presumably arise from the processing of the longer tRNA transcript, represent a novel and potentially important group of ncRNAs. Currently, the knowledge about the biogenesis of these fragments, their roles, and their potential function remains limited.

Studies with human cell lines have shown that tRNAs can be cleaved at the anticodon loop to produce “tRNA halves” that are (30-35 nts in length) a process that seems to be facilitated by the enzyme, Angiogenin, following induction of stress. Referred to as “tRFs,” tRNA fragments have also been found to originate from cleavage of either the mature tRNA or the tRNA precursor molecule. In the latter case, RNase Z cleaves the 3′ part of the tRNA precursor as part of the maturation process and the resulting fragment is considered to be a tRF with reported functions. tRFs that are derived from mature tRNAs emerge after cleavage at either the D-loop (giving rise to 5′-tRFs) or the T-loop (giving rise to 3′-tRFs with the CCA addition present) and are about 20 nucleotides long. Further investigation into the enzymes responsible for the fragments has shown that the process is Dicer-dependent, Angiogenin-dependent (cleaving the tRNA at the T-loop) or RNase-Z-dependent (producing 5′-tRFs).

tRFs are likely not random degradation products. Some 3′-tRFs are loaded on Argonaute, thereby, admitting a miRNA-like behavior and are involved in regulation of gene expression affecting physiological processes like cell growth, cell proliferation and cellular responses to DNA damage. These fragments have also been shown to have regulatory roles in translation initiation and stress granule formation, so it is reasonable to anticipate that additional functions await discovery. 3′-tRFs have also been described to emerge in human MT4 T-cells after HIV infection from the host cell.

Further adding to the likelihood that they are not random in nature is the fact that tRFs have been described in mouse, a yeast, two protozoans, a bacterium, and an archaeon. As described herein, the presence of tRNA fragments is discussed in different human tissues.

Unlike previous studies that focused on 5′-tRFs, 3′-tRFs, and on tRFs overlapping with the 3′ of the precursor tRNA transcripts in what follows, no restrictions were imposed on the sought fragments in terms of their relative position with respect to the span of a mature tRNA. tRNA fragments were systematically studied by analyzing 452 short RNA profiles from lymphoblastoid cell lines and 311 breast samples from The Cancer Genome Atlas (TCGA) repository at the National Institutes of Health (NIH).

Four previously known categories were observed, namely 5′-tRFs, 3′-tRFs, 5′-halves and 3′-halves, in the two human tissues types (all previous reports were based on model cell lines). A fifth structural category of tRFs was found, the internal-tRFs or “i-tRFs,”in human tissues. The i-tRFs begin and end anywhere along the interior of the mature tRNA. The i-tRF category was found to be rich and diverse, with numerous tRNA genomic loci producing many distinct i-tRFs. The 5′-tRF and 3′-tRF categories were found to be more diverse than previously thought: each contained multiple tRFs with distinct, quantized lengths that changed from tissue to tissue, and between health and disease.

The tRFs from nuclear tRNA loci were also found to differ greatly from mitochondrial (mt or MT) tRFs. Within a tissue, the lengths and abundances of 5′-tRFs, i-tRFs, and 3′-tRFs depended on the genomic origin (i.e. nuclear vs. mitochondrial) of the parent tRNA locus. Notably, this observation held true even for tRFs from the same anticodon, i.e. nuclear AspGTC vs mitochondrial AspGTC.

The tissue type and disease type/sub-type appeared to shape the tRF population. All tRF categories exhibited diversities and abundances that were tissue-specific, specific between health and disease, and dependent on disease subtype.

Moreover, it was surprising to find that gender, population, and enthicity shaped the tRF population. In fact, the tRFs had gender-, population- and ethnicity-dependent differences at the molecular, cellular, and tissue levels, in healthy and diseased tissues.

The tRFs also loaded on Argonaut (Ago) in a cell-line-specific manner. The analyses of Ago CLIP-seq data from cell lines modeling three BRCA subtypes showed that different populations of tRFs were Ago-loaded in each cell line.

The i-tRFs were also present in clinical samples. Using sequence-specific amplification methods, the presence of several novel molecules was independently confirmed in clinical samples from BRCA patients.

The analysis of tRNA sequences required a number of special considerations that went beyond what is typically done when mapping RNA-seq data for the purpose of e.g., profiling the expression of miRNAs or mRNAs. These considerations stemmed from the observations that tRNAs are in fact repeat elements. In addition to bonafide tRNAs having multiple copies, tRNA segments appeared elsewhere on the genome outside of tRNA space, the latter being the full complement of genomic locations harboring tRNA genes.

Method for Identifying Bona Fide tRNA Fragments from Deep Sequencing Transcriptomic Data

The proper analysis and handling of tRNA sequences and identification of tRNA fragments in deep sequencing data required several considerations that went beyond typical protocols to map RNA-seq data for the purpose of e.g., profiling the expression of miRNAs or mRNAs. These considerations stemmed from the fact that tRNAs are repeat elements. Considering the logical hierarchy that pertains to tRNA sequences, where each amino acid (few amino acids; at the top of the pyramid) has multiple associated anticodons (more isoacceptors than amino acids; in the middle of the pyramid) and each anticodon has multiple associated genomic instances (a multitude of isodecoders; at the bottom of the pyramid), the presented analyses sought to unravel, for each tRNA fragment, details at the lowest possible level of the hierarchy. It is stressed that the nature of the sequences at hand is such that achieving this goal is unattainable in some instances. As many isodecoders have indistinguishable sequences, results were reported at the anticodon level, an intermediate level between isoacceptors and isodecoders. Also, the method kept track of the genomic origins of all the reported fragments.

Defining the tRNA Space

All shown sequences are in 5′→3′ orientation. Directly linked to the mapping is the definition of what constitutes the genome's tRNA space. For the purposes of the method described herein, the following sequences were combined to make up the tRNA space:

a) the 22 known human mitochondrial tRNA sequences (NCBI entry NC_012920.1

b) 610 (508 true tRNAs and 102 pseudo-tRNAs) of the 625 nuclear tRNA sequences from the genomic tRNA database (GtRNAdb); excluded from the 625 nuclear tRNAs listed in GtRNAdb were the entries that included the selenocysteine tRNAs, tRNAs with undetermined anticodon identity, and tRNAs mapping to contigs that were not part of the human chromosome assembly;

c) the eight genomic intervals chr1:+:566062-566129, chr1:+:568843-568912, chr1:−:564879-564950, chr1:−:566137-566205, chr14:+:32954252-32954320, chr1:−: 566207-566279, chr1:−:567997-568065, and, chr5:−:93905172-93905240—all coordinates were from the hg19 assembly of the human genome—that corresponded to identical instances of seven mitochondrial tRNAs TrpTCA, LysTTT, GlnTTG, AlaTGC (×2), AsnGTT, SerTGA, and, GluTTC, respectively.

In total, the reference tRNA space used to implement the method described herein included 640 sequences.

Sequenced Reads were Mapped “Exactly” Since Mapping with Mismatches Generated Erroneous Results

Multiple sequence alignments of the genomic copies for a given anticodon revealed many instances of sequence segments that were shared among these copies and made to look like one another if one permitted a small number of insertions, deletions, or replacements. These segments were found across the length of the mature tRNA, and were present in tRNA fragments. Moreover, these segments were found in the sequences of distinct anticodons of the same amino acid. Consequently, permitting insertions, deletions or replacements during read mapping misidentified the genomic origin of a read and led to erroneous results. Problems occurred even if indels were excluded and a single replacement allowed.

For example, the following 5′-tRF sequence GGGGAATTAGCTCAAG-T-GGTAGAGCGCTTGCT (SEQ ID NO:55729) appeared at five genomic locations, all of which were instances of the AlaAGC anticodon sequence.

By contrast, the 32 nt sequence GGGGAATTAGCTCAAG-C-GGTAGAGCGCTTGCT (SEQ ID NO:55730), differed from the previous sequence at a single location (T→C), a 5′-tRF of AlaAGC, but appeared at two different genomic locations that were distinct from the previous five. If read mapping with a single mismatch was allowed, these two distinct 5′-tRF molecules would become indistinguishable, thereby, confounding any transcriptional differences that potentially exist among the seven full length loci that comprise GGGGAATTAGCTCAAG-N-GGTAGAGCGCTTGCT (SEQ ID NO:55731).

This problem was accentuated further when the typically shorter reads contained in “short RNA-seq” datasets were mapped. The 22 nt sequence GGGGGTGTAG-A-TCAGTGGTAGA (SEQ ID NO:55732) is a 5′-tRF from the AlaAGC anticodon (trna117 on chromosome 6). Allowing for exactly one mismatch made this 5′-tRF indistinguishable from GGGGGTGTAG-C-TCAGTGGTAGA (SEQ ID NO:55733), which appeared in 11 isodecoders of three Ala anticodons (AlaAGC, AlaCGC, AlaTGC) as well as in two non-Ala anticodons, namely CysGCA (trna7 on chromosome 3) and ValAAC (trna115 on chromosome 6). Thus, if a single replacement was allowed during mapping, reads from any one of these 14 genomic locations were indistinguishable, and led to cross-talk and consequent erroneous estimates about the abundance of 5′-tRFs arising from the tRNAs. To avoid such confounding events, insertions, deletions, or replacements were not permitted.

Sequenced Reads were Mapped on the Full Genome (Nuclear and Mitochondrial) Since Mapping on tRNA Space Alone Generated Erroneous Results

It was tempting to consider compiling a database of tRNA sequences (e.g. by combining all the spliced nuclear and mitochondrial tRNA sequences into a single collection) and then mapping the sequenced reads on this subset of the genomic real estate. Such an approach would be easy to implement, fast to execute, and seemingly adequate. Unfortunately, this approach was error-prone and led to misrepresentation of expressed tRNA fragments and miscalculation of the relative abundances of the various tRNA anticodons.

In addition to the multiple instances of bonafide nuclear tRNAs, the human genome is also riddled with many instances of nuclear and mitochondrial tRNA-lookalikes, as well as partial tRNA sequences. Thus, any and all reads that simultaneously land inside and outside tRNA space were excluded from consideration since their integrity could not be guaranteed.

To achieve this objective all sequenced reads were mapped on the entire genome. The 24 nt sequence GCTCCAGTGGCGCAATCGGTTAGC (SEQ ID NO:55734) helped illustrate the reasoning for this requirement. The sequence is a 5′-tRF of the IleTAT anticodon and appeared identical to five genomic locations. However, this sequence also appeared outside tRNA space on the forward strand of chromosome 7, between locations 44465584 and 44465607 inclusive (GCh37). This sequence forms part of the 38 nt sequence GCTCCAGTGGCGCAATCGGTTAGCATGCGGTACTTATA (SEQ ID NO:55735) (note the underline segment) that spans locations 44465584 through 44465621. Even though this 38-mer is labeled as a “tRNA” by RepeatMasker, it is much shorter than the 93 nt of the typical IleTAT and thus not a bonafide tRNA. Consequently, the sequenced reads were mapped on the whole genome, which allowed the identification of such events. Since the integrity of the reads that fall in this category could not be established unambiguously, all reads that landed simultaneously inside and outside tRNA space were discarded and excluded from further consideration.

Sequenced Reads were Mapped Using Exact Multi-Mapping Since Mapping Uniquely Generated Erroneous Results

Typical pipelines that map deep-sequencing datasets report reads that can be mapped either unambiguously to a single location (“unique-mapping”) or only to handful of genomic locations. However, considering that the typical tRNA anticodon has multiple genomic instances, neither of these two choices was appropriate under the circumstances.

As an example, the 72 nt AspGTC sequence TCCTCGTAGTATAGTGGTGAGTATCCCCGCCTGTCACGCGGGAGACCGGGGT TCGATTCCCCGACGGGGAG (SEQ ID NO:55736) was presented. This sequence appeared identically at 11 genomic loci: five on chromosome 1, two on chromosome 6, three on chromosome 12 and one on chromosome 17. The typical read for short RNA-seq profiles was shorter than 72 nt, which increased the chance that a read was present at multiple genomic locations some of which were not related to tRNAs. The multiple instances of tRNA anticodons, the existence of the previously reported tRNA lookalikes, and the existence of repeating elements, like the previously reported pyknons, required “exact multi-mapping” (i.e. no indels, no replacements) to be carried out. A sequenced read was permitted to map as many locations as practically possible. The resulting maps were post-processed and any sequenced reads with one or more of instances outside the tRNA space were discarded and excluded from further consideration. On the other hand, sequenced reads with all of instances exclusively inside tRNA space were kept. In an example case, the method only read counts from one of the multiple genomic loci to avoid mis-counting the fragment's abundance.

Sequenced Reads were Mapped “Exactly” while Accounting for the Nontemplated CCA Addition

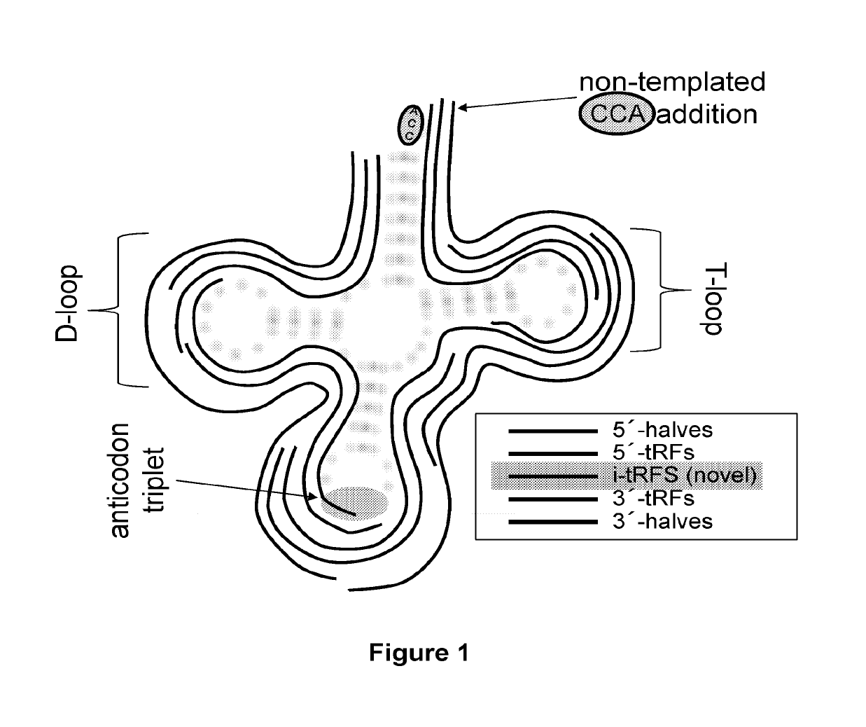

As shown in FIG. 1, mature tRNA sequences contain the CCA trinucleotide that is added post-transcriptionally to the 3′ end of mature tRNAs. Since this CCA “tail” of the mature tRNA has no counterpart in the genomic DNA, from which the precursor tRNA sequence was transcribed, explicit provisions were made to map such reads or they would be inadvertently excluded from consideration and from reporting. These provisions were necessary since the method presented herein required a strict-exact mapping of the sequenced reads. Consequently, the nontemplated CCA's presence in a read could not simply be accommodated by allowing an adequate number of mismatches (e.g. replacements) during mapping. Prior to mapping, a modified instance of the genome was created where the trinucleotide CCA was used to replace the three genomic nucleotides immediately downstream (in the 5′→3′ direction) of each of the 640 reference mature tRNAs.

Special bookkeeping was required in the case of mitochondrial tRNAs, some of which are either very close to one another (e.g. MT_AlaTGC and MT_AsnGTT) or overlapping (e.g. MT_CysGCA and MT_TyrGTA). A careless replacement of the genomic sequence downstream from a tRNA by the CCA trinucleotide would inadvertently “erase” part of another tRNA's sequence.

Lastly, it was important to realize that depending on its length, a sequenced read that ended in CCA could simply be a transcript that originated elsewhere on the genome from a location that was outside tRNA space. In such an event, the reads that fall in this category could not be established unambiguously and these particular CCA-ending reads were discarded and excluded from further consideration.

Sequenced Reads were Mapped Using Special Provisions for Intron-Containing tRNAs

An abundance of tRNAs contains introns. The method described herein focuses on mature tRNAs. Sequenced reads that mapped on the genome under the above constraints yet straddled a tRNA exon-intron or a tRNA intron-exon boundary were discarded and excluded from further consideration. At the same time, the sequence space on which reads were mapped needed to be augmented to include spliced versions of all intron-containing tRNAs.

However, attention was required as follows: a) mapped reads that were wholly in an exon of an intron-containing tRNA were counted once (e.g. only their genome instances were counted and not their spliced-tRNA instances; or vice versa); and, b) mapped reads that straddled a tRNA exon-exon junction were examined for possible instances outside tRNA space. The mapped reads that straddled such a junction but also had instances outside tRNA space were discarded and excluded from further consideration.

As a specific example of a tRNA fragment that highlighted the need for this step, the following TyrGTA sequence is described. The tRNA fragment's sequence comprises the tail of the first exon and the head of the second exon, which indicates that the fragment arose from a mature or semi-mature tRNA molecule. Specifically, the 19-nucleotide fragment trna14_nTyrGTA_6_+_26569086_26569176@32.50.19_1_0_12 is an internal fragment that maps solely on the 12 genes of the nuclear TyrGTA anticodon and spans the exon-exon junction in all 12 cases. This 19-mer, which does not appear elsewhere in the genome, would have been discarded if special provisions were not made for handling tRNA introns.

Distinguishing Among Three Regions of the Mature tRNA that were Sources of tRNA Fragments

For each of the considered tRNAs, and for the reads with instances exclusively in tRNA space, three categories of tRNA fragments were identified that arose from three regions of the tRNA: a) fragments whose 5′ terminus began exactly at the 1^{st} nucleotide of the corresponding mature tRNA (“+1” fragments; the category comprises 5′-tRFs and 5′-halves); b) fragments that were strictly internal to the mature tRNA sequence, i.e. whose 5′ terminus began at the 2^{nd} nucleotide or further to the right and whose 3′ terminus ends to the left of the first “C” of the nontemplated “CCA” addition to the mature tRNA (“internal” fragments or i-tRFs); and, c) fragments whose 3′ terminus coincided with any of the bases of the “CCA” terminal addition (“CCA-ending” fragments; the category comprised 3′-tRFs and 3′-halves). It was also recognized that there were instances of mature tRNAs, e.g. the histidine (His) tRNA, that gave rise to fragments that started at the “−1” position i.e. one position to the left of the start of the mature tRNA. For simplicity of presentation, these were considered subsumed by the “+1” region and were not treated separately.

The categories of fragments starting at position +1, and the ones ending at the CCA tail have been described previously. However, until now, i-tRFs had not been described as a distinct and rich category of abundant tRFs, in either cell lines or in human tissues.

### Analyzed Datasets

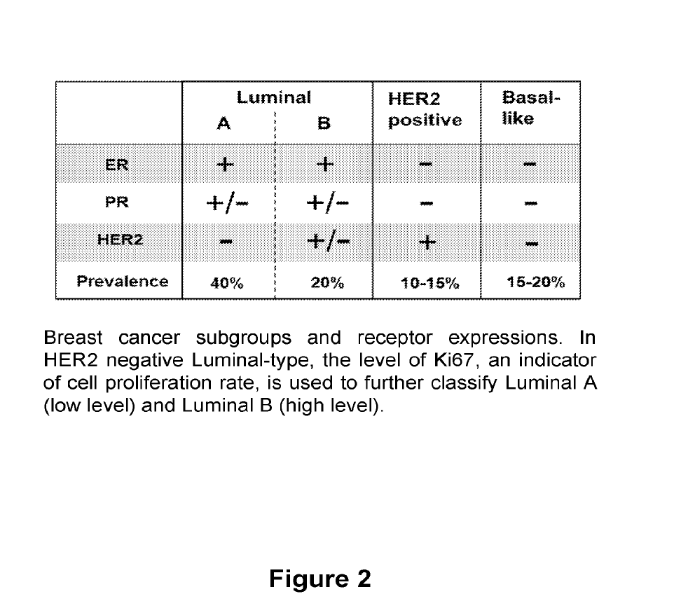

The first analyzed dataset contained the short-RNA sequencing profiles of lymphoblastoid cell lines (LCLs) from 452 men and women belonging to five different populations: Utah residents with Northern- and Western-European ancestry (CEU), Finnish (FIN), British (GBR), Toscani Italians (TSI) and Yoruba African from the city of Ibadan (YRI). The second analyzed dataset was drawn from The Cancer Genome Atlas (TCGA) repository at the National Institutes of Health (NIH) and comprised 17 normal and 294 breast cancer samples covering the basic hormone profiles (FIG. 2).

In what follows, LCL refers to both the analyzed 452 primary datasets and the corresponding collection of 1,113 statistically significant tRNA fragments. Analogously, BRCA is used to refer both to the analyzed 311 primary datasets and the corresponding collection of statistically significant tRNA fragments.

Sequenced reads were mapped as mentioned above and all tRNA fragments that were supported by at least one read in at least one of each collection's analyzed samples were collected. Then, filtering criteria were applied that ensured that each tRNA fragment had enough statistical support. For the LCL dataset, the filtering led to 1,113 statistically significant tRNA fragments, SEQ ID NOs: 24833-25945. For the BRCA dataset, the filtering led to 315 statistically significant tRNA fragments, SEQ ID NOs: 8538-8852. For the Brain dataset, the filtering led to 1802 statistically significant tRNA fragments, SEQ ID NOs: 1-1802. For the CLL dataset, the filtering led to 2014 statistically significant tRNA fragments, SEQ ID NOs: 12462-14475. For the Pancreas dataset, the filtering led to 1367 statistically significant tRNA fragments, SEQ ID NOs: 36100-37466. For the Platelets dataset, the filtering led to 508 statistically significant tRNA fragments, SEQ ID NOs: 51286-51793. For the Prostate dataset, the filtering led to 1373 statistically significant tRNA fragments, SEQ ID NOs: 42349-43721.

Breast cancer is the most frequently diagnosed cancer and the leading cause of cancer death among women. In the United States, one in eight women will develop breast cancer during her lifetime. In 2013 alone, nearly 300,000 individuals were diagnosed with either invasive or non-invasive breast cancer whereas 40,000 died from breast cancer. Breast cancer is also a heterogeneous disease. The discovery of specific prognostic and predictive biomarkers in the past decades has enabled the clinical classification of breast cancer into three basic therapeutic subgroups (FIG. 2). The estrogen receptor (ER) positive group, known as Luminal-type breast cancer, represents the most frequently occurring and diverse type, and its treatment often includes endocrine therapy. The Basal-like subgroup, also termed “triple-negative”, lacks transcription from either the ER, the progesterone receptor (PR) or the epidermal growth factor receptor 2 (HER2) locus: this is a group with poorer prognosis and chemotherapy is the only option in this case. It is standard practice for pathological features (e.g. hormone receptor positivity, tumor stage and node positivity) to be used to guide clinicians in prescribing the appropriate therapy. Sustained efforts in understanding further the molecular etiology behind the onset and development of breast cancer are necessary before improved biomarkers of risk and of therapy response can be developed and applied towards higher-quality diagnosis and treatment approaches.

Exact Multi-Mapping Reveals Atypical tRNA Length Fragments

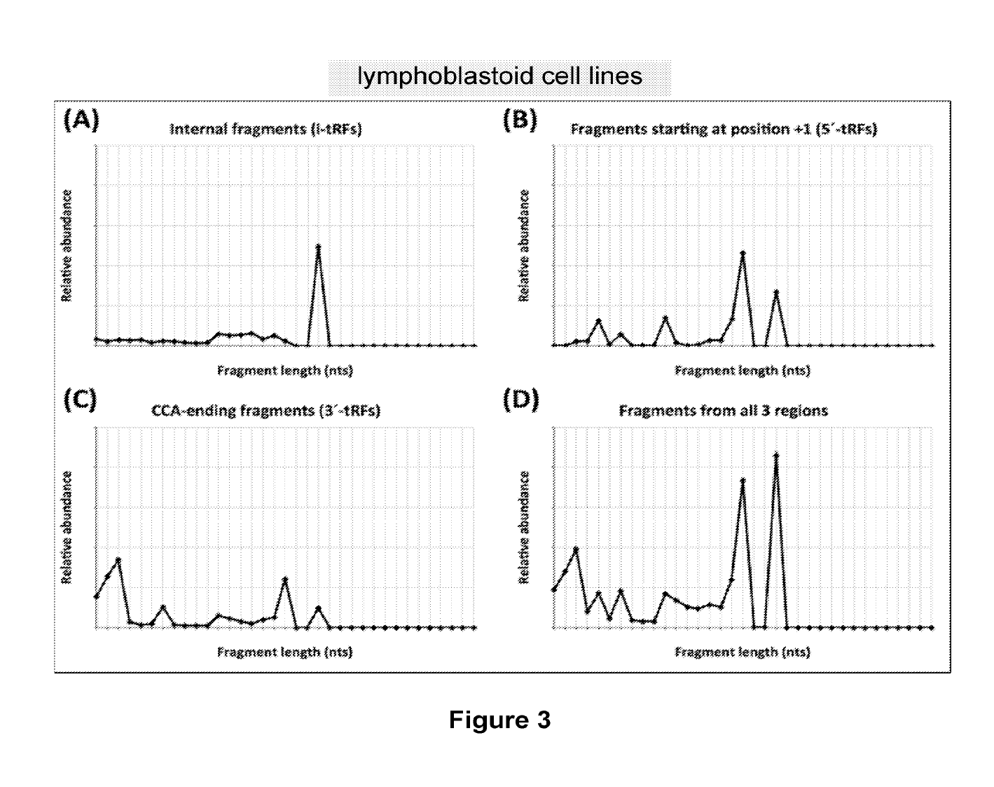

The lengths of the reads mapping to the internal region were plotted on a histogram and compared to the two known categories of tRNA fragments (5′-tRFs and 3′-tRFs). FIGS. 3A-3D show the length distributions for the 452 individuals of the LCL dataset. As can be seen from FIG. 3A, i-tRFs are dominated by a single length, namely 36 nt. The 5′ terminus of the i-tRFs begins at position +2 of the mature tRNA, or further to the right. Consequently, the internal 36-mers comprise the full anticodon triplet (typically centered at position +34 of the mature tRNA sequence) and thus they straddle the point that has been typically associated with the terminus of tRNA halves.

FIGS. 3B-3C show the length distributions for the +1 and the CCA-ending regions. In FIG. 3D, the combined length distribution is shown. Each of the three tRNA regions gave rise to fragments with characteristic length profiles and specific relative abundances. Importantly, the very small standard errors (too small to be visible in the four panels) indicate that the lengths of these fragments persisted across each of the three regions and across the 452 individuals and, thus, were not random degradation products.

In the tRNA literature, the 5′-tRFs have been associated with lengths of 18, 22, and 32 nt. In addition to identifying fragments with these lengths, the analysis of the LCL datasets revealed a prevalence for fragments with lengths of 20, 26, 33 and 36 nt. These lengths have not been previously associated with 5′-tRFs.

Similarly in the LCL datasets, the CCA-ending fragments (3′-tRFs) show prevalence for lengths of 18, 22, 33 and 36 nt. More than half of these 33-mers and 36-mers start after the anticodon, which makes many of these fragments distinct from the typical tRNA-halves and complementary to the previously reported length-families of 3′-tRFs. It is also worth noting that all the 3′-tRF 33-mers and more than half of the 3′-tRF 36-mers (26 out of 43) originated in mitochondrial tRNA genes.

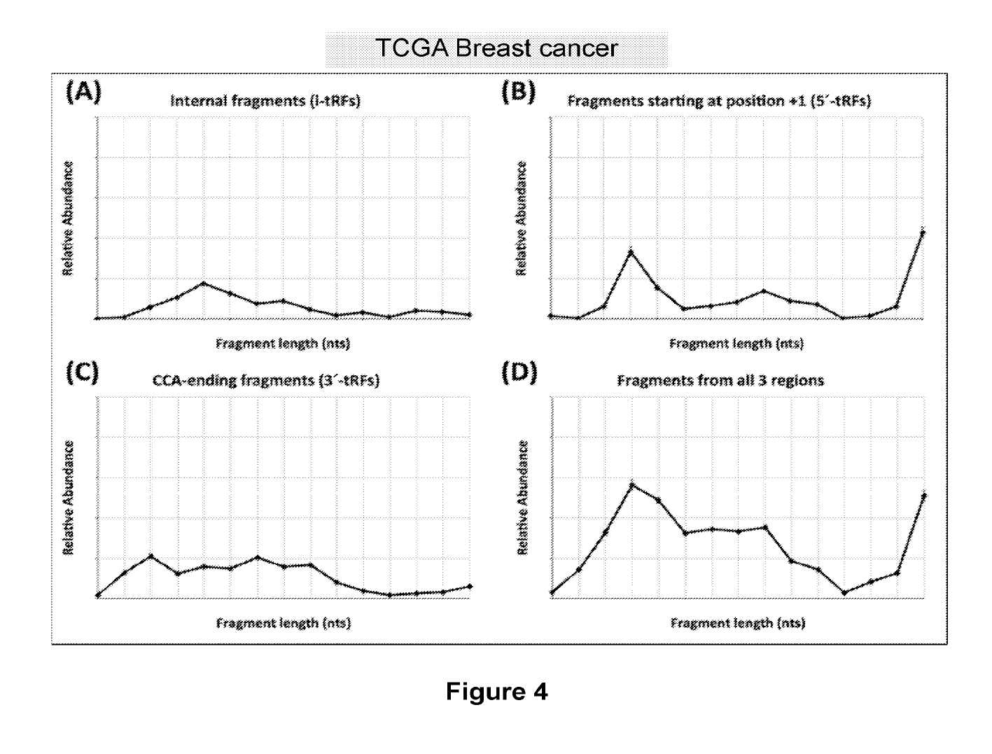

The same analysis was repeated for the 311 TCGA BRCA datasets. FIG. 4A-4D show the corresponding length distributions. The i-tRF distribution (FIG. 4A) is significantly different from those of the 5′-tRFs (FIG. 4B) and the 3′-tRFs (FIG. 4C). The i-tRFs comprise fragments that are 20 nt long and virtually no fragments 230 nt, whereas the +1 category is characterized by a prevalence of fragments with lengths 19, 20, 24 and ≧230 nt. Similar to the LCLs, the lengths of the fragments that arise from the three regions have characteristic profiles and specific relative abundances.

Moreover, the small standard error for each length indicates that atypical lengths of these fragments are rare across the analyzed datasets. It is important to emphasize that these NIH-TCGA datasets were obtained through deep sequencing PCR with a total of 30 sequencing cycles. Consequently, short fragments or fragments longer than 30 nt that may exist in each sample's milieu were represented by a 30-mer “proxy”.

A considerable portion of the CCA-ending fragments in the BRCA datasets have lengths that have not been previously associated with 3′-tRFs. In all, these datasets revealed several length families that have not been previously reported. These families comprise fragments with lengths of 16, 20, 21, and 23-29 nt and collectively account for 21.2% of all tRNA fragments in the BRCA datasets. In FIG. 4D, the combined length distribution is shown.

i-tRFs Represent a Diverse New Family of tRNA Fragments

The analyses described herein reveal that i-tRFs are a surprisingly rich category with many of its members having 5′ termini that are away from the 5′ end of the mature tRNA. The i-tRFs represented 27.5% of all fragments in the LCL and 21.0% of all fragments in the BRCA dataset.

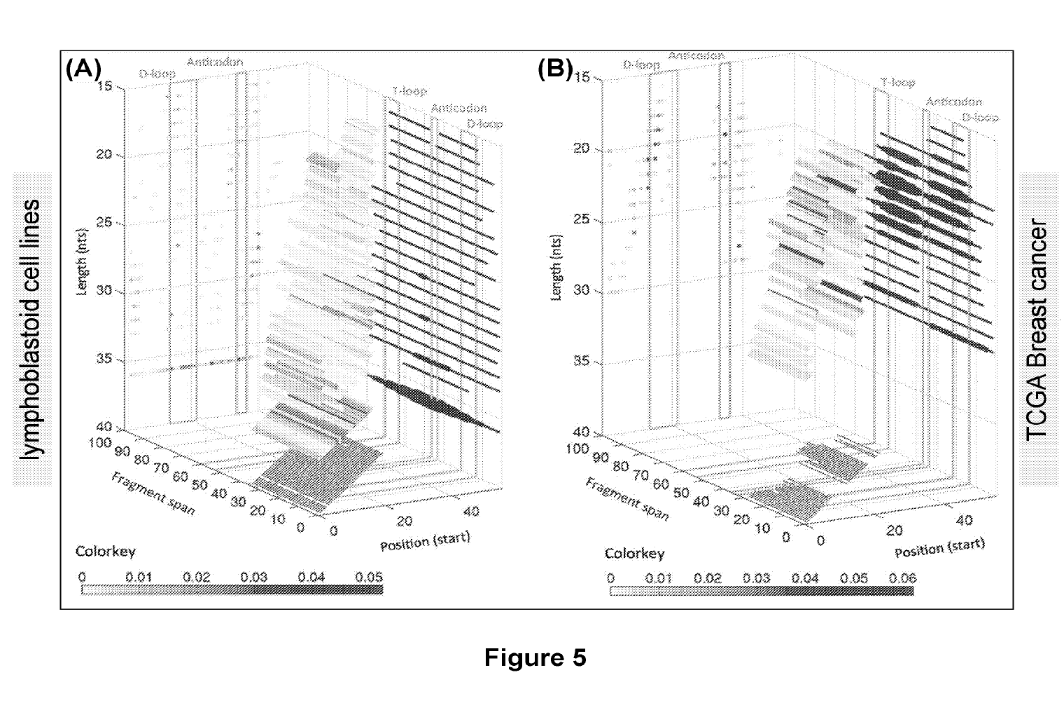

FIG. 5 shows the distribution of the starting positions of the i-tRFs for the LCL (A) and BRCA datasets (B). For each starting position, the length distribution is also shown as bars, with the intensity of each bar representing the average expression of the respective fragment in the LCL or BRCA dataset.

For the LCL dataset, internal 36-mers began anywhere within the D loop of the mature tRNA (generally positions 12-22) or immediately after it (in 5′→3′ orientation). No specific position is singled out as the preferred starting position of internal fragments in this dataset (FIG. 5A). On the contrary, in the BRCA dataset, there are two main “clusters” of starting positions for the i-tRFs: a first cluster spanning positions 11-17 that generally reside in the D loop and a second cluster spanning positions 32-43 comprising the anticodon loop and the variable loop of the mature tRNA (FIG. 5B). Each of the starting positions exhibited its own associated range of lengths for the fragments that began there. Fragments that began at position 13 were 23, 22 or 21 nt long, whereas fragments that began at position 15 or 16 were slightly shorter with lengths 19, 20, or 21 nt.

These fragment lengths recurred in both the LCL and BRCA datasets and have small standard deviations. It is thought that the mechanisms behind the production of these fragments have specific preferences for the starting and ending positions and/or the length of the tRNA fragment. The 30-mers in FIG. 2B, starting at positions 2-6, 34 and 36, are likely tRNA-halves that cannot be “seen” due to the 30-PCR-cycle limitation in the breast datasets mentioned herein.

tRFs Differ Between Nuclearly- and Mitochondrially-Encoded tRNAs

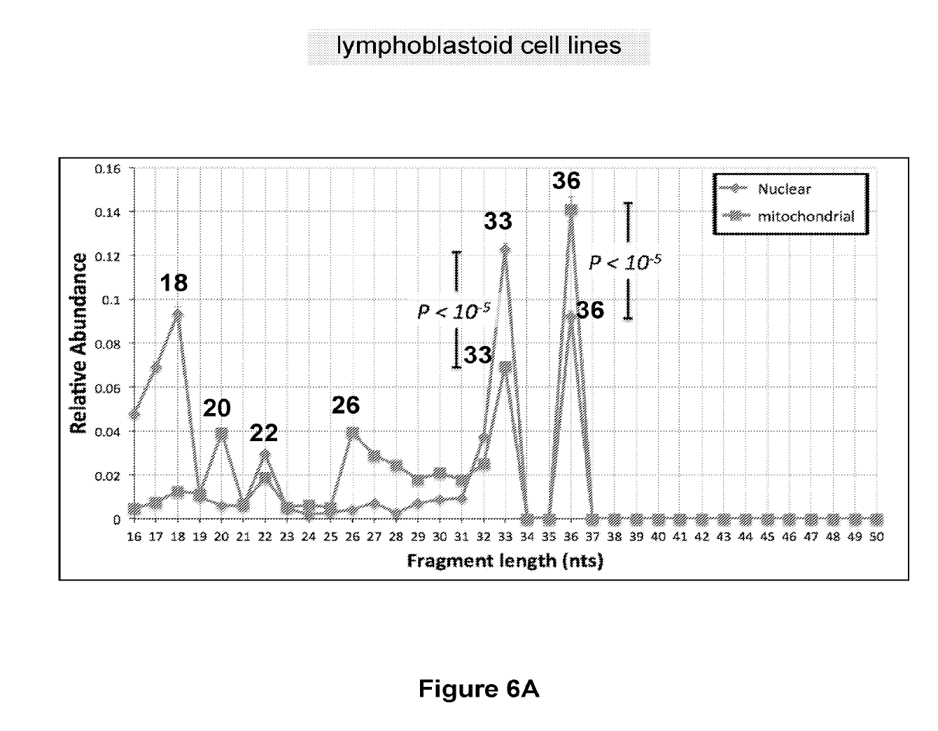

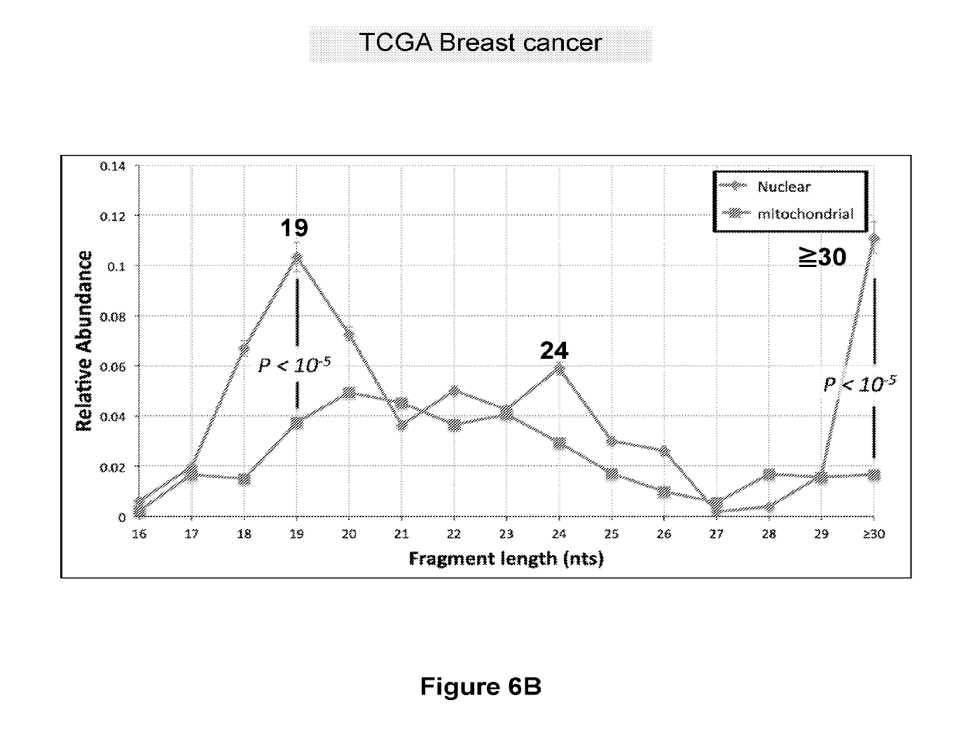

The relationship between tRNA fragment lengths and abundances, and their genomic origin (i.e. whether nuclearly-encoded vs. mitochondrially-encoded) was examined. To this end, the graphs of FIGS. 3D and 4D were decomposed into their nuclear and the mitochondrial contributions (FIG. 6A-6B). Several statistically significant differences were identified in the expression of nuclear and mitochondrial tRNAs in both the LCL (A) and the BRCA dataset (B). Notably, the 36-mers in the LCL dataset were predominantly from mitochondrially encoded tRNAs, while the 33-mers were from nuclearly encoded ones.

tRFs from all Three Regions Exhibit Diversities and Abundances that Depend Strongly on the Choice of Anticodon

For each of the two collections of analyzed datasets, and separately for each anticodon, the fragments arising from all of the bonafide genomic instances of the anticodon being considered each time were enumerated. In each case, the number of fragments arising from each of the three regions of the mature tRNA, namely “+1”, “internal,” or “CCA-ending” was determined. The fragments originating from pseudo-tRNAs and from sequences of potential pseudo-tRNA origin were also enumerated and found to be considerably fewer than those from true tRNAs.

In the LCL collection, 63 anticodons (from a possible total of 75 nuclear and mitochondrial ones) that generated fragments with abundance levels that meet the mapping and filtering criteria were found. The mitochondrial tRNA GluTTC generated the highest number of distinct tRNA fragments followed by the nuclear LysCTT. Notably, the diversity of fragments that arose from each of the three regions of the mature tRNA strongly depended on the anticodon at hand. For some anticodons, the “+1” region gave rise to the most diverse set of tRNA fragments (e.g. nuclear GluTTC), whereas for other anticodons most of the diversity was encountered in the internal (e.g. mitochondrial HisGTG) or the CCA-ending regions (e.g. mitochondrial ValTAC).

Analogously, in the BRCA collection, 52 of the 75 possible nuclear and mitochondrial anticodons generated fragments satisfying the filtering criteria. As with the LCL datasets, the diversity of fragments that arose from each of the three regions of the mature tRNA strongly depended on the considered anticodon. Similarly to the LCL collection, the mitochondrial GluTTC produced the highest number of distinct fragments as well, whereas the mitochondrial ValTAC gave rise mainly to CCA-ending fragments.

The analysis of these two different types of datasets also revealed examples of anticodons where the fragment profile changed with the tissue type (see also below). For example, in the LCL datasets, the nuclear AlaACG generated predominantly CCA-ending fragments. On the other hand, in the BRCA datasets the anticodon's 5′-tRFs were favored as well and were produced at a ratio of 1:1 compared to the 3′-tRFs.

Additionally, the abundance of the tRNA fragments exhibited anticodon-dependencies as well. In fact, from this standpoint the differences between the LCL and the BRCA collections were more pronounced. In the LCL dataset, the relative abundances of different fragment lengths were due to fragments from different anticodons. For example, the mitochondrial SerGTC anticodon was responsible for 68.7% and 80.4% of the contribution to fragments with the previously unreported lengths of 20 and 26 nt. On the other hand, for fragments of length 36 nt, it was the nuclear GluCTC, nuclear GluTTC, and the mitochondrial GluTTC anticodons that accounted for 37.9% of all 36-mers, with the rest being contributed by an assortment of anticodons. Interestingly, in the BRCA datasets, the mitochondrial ValTAC anticodon generated approximately 30.0% of the fragments with lengths of 20-23 nt.

The Fragments Arising from Different Regions of the Same Anticodon have Uncorrelated Abundances

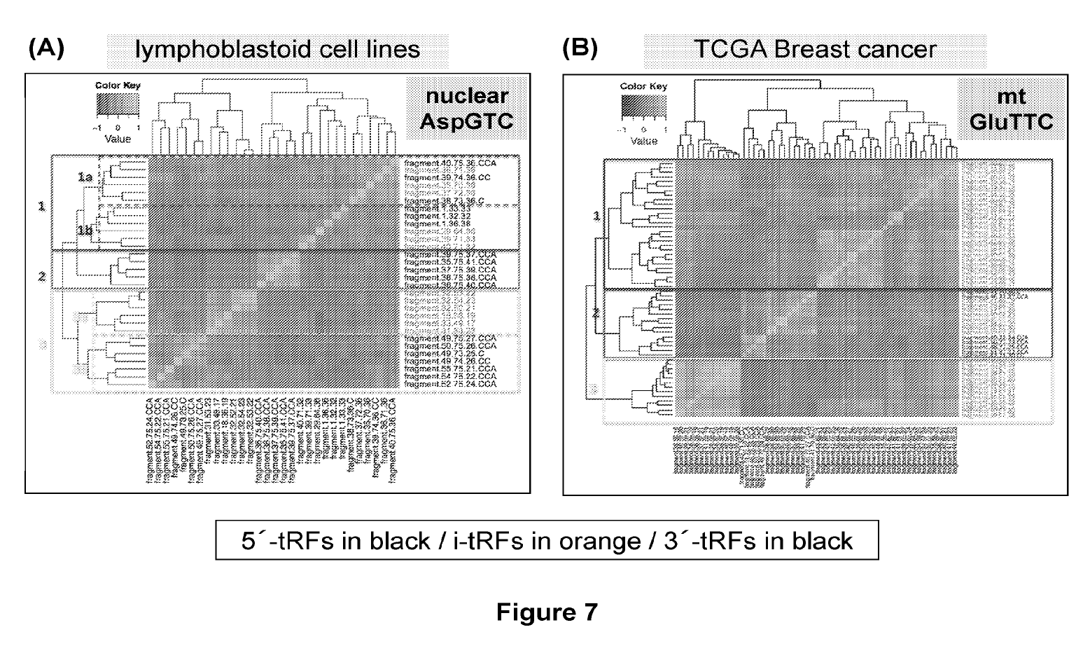

Considering the richness of fragments that can arise from a given anticodon, it was investigated whether the abundances of the fragments were correlated. FIG. 7A shows a Pearson correlation heatmap for the fragments that arose from the nuclear AspGTC in the LCL datasets. FIG. 7B shows the analogous heatmap for the mitochondrial GluTTC in the BRCA datasets. This anticodon produced the largest number of fragments in the BRCA datasets and most of them were internal, i.e. i-tRFs. The abundances of reads originating from the three tRNA regions (i.e., “+1,” “internal,” “CCA-ending”) have a poor correlation.

A poor correlation also characterizes the fragments that arise from the same anticodon, yet are of different lengths. Several small clusters of poorly correlated regions are apparent in the heatmaps.

For the nuclear AspGTC (LCL datasets—FIG. 7A), cluster 1a comprises internal and CCA-ending 36-mers, whereas cluster 1b captures mainly internal 32-mers and 33-mers. Cluster 2 comprises CCA-ending fragments that are 37 nt or longer. Cluster 3b contains CCA-ending fragments between 24 and 27 nt, whereas cluster 3c comprises internal fragments between 17 and 23 nt.

Analogous observations can be made for the fragments for the mitochondrial GluTTC fragments (BRCA datasets—FIG. 7B). Short internal fragments, generally of length 21 nt or shorter, form cluster 3, while internal fragments of intermediate length (21-27 nt) comprise cluster 1. On the other hand, cluster 2 contains long internal fragments and all of the CCA-ending fragments from this anticodon. A mini sub-cluster of cluster 2 comprises shorter CCA-ending fragments (22-25 nt).

Examination of the Pearson correlation maps for the other anticodons shows that they are qualitatively similar to the ones shown in the two panels of FIG. 7. Two general observations are apparent. First, evidence was found in all anticodons for well-defined mini-clusters, each containing only a few of the anticodon's fragments. The members of each such mini-cluster had correlated abundances.

Second, when a given anticodon's mini-clusters was compared with another, a characteristic absence of correlation was observed, even in cases where fragments from two mini-clusters overlapped on the mature tRNA sequence from which they originated (see, for example, the mini-clusters 1a and 2 in FIG. 7A, or clusters 1 and 3 in FIG. 7B). These observations, in conjunction with the small standard error across the 452 (LCL) and 311 (BRCA) individuals shown in FIG. 1, lend more weight to the view that the fragments from all three regions of the mature tRNA are constitutive in nature and not random degradation products.

The tRNA Fragments have Lengths that are Specific to Tissue and Tissue-State

Inspection of the distributions shown in FIGS. 3 and 4 indicates that the specifics of 5′-tRFs, i-tRFs, and 3′-tRFs depend strongly on the tissue. Looking at the BRCA datasets (and without distinguishing between the normal and tumor datasets), it was evident that the dominant fragments have lengths between 19 and 24 nt and account for 60.2% of all tRNA fragments in this collection. By contrast, the LCL datasets have dominant fragments with lengths of 18, 33, and 36 nt and account for nearly 50% of all tRFs.

To increase the resolution, the BRCA fragment distributions of FIG. 4A-4D were further decomposed into their two constituent parts, namely the subset of normal datasets and that of the tumor datasets (FIG. 4).

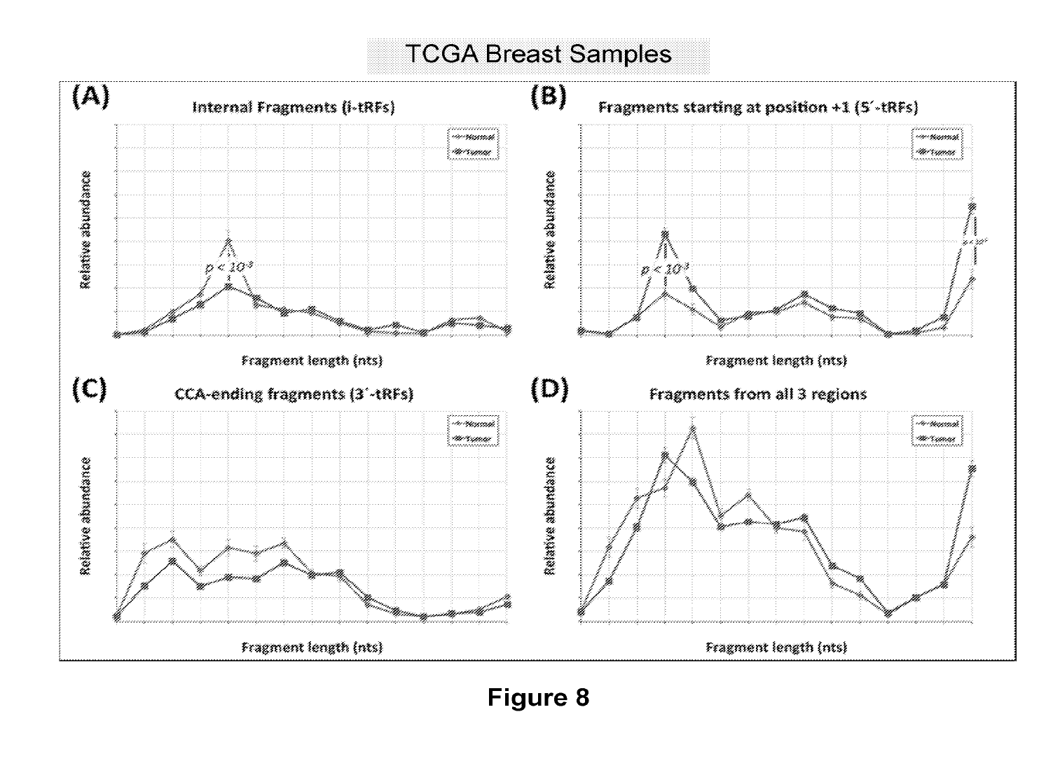

The tissue-type differences that existed between the normal BRCA and the normal LCL datasets were now more evident. In the internal region, 36-mers i-tRFs were the lion's share in the LCL set (FIG. 3A), whereas in the BRCA set, 20-mer i-tRFs provided a modest contribution to the total pool of fragments in the normal breast datasets (FIG. 8A).

In the +1 region, 5′-tRFs with length 19 nt (FIG. 8B) were the dominant population in normal breast (compare this with the 33-mers and 36-mers in the +1 region in LCLs, shown in FIG. 3B).

Lastly, the CCA-ending region was dominated by 17-mer, 18-mer and 33-mer 3′-tRFs in LCL (FIG. 3C), yet a fairly uniform distribution was found in i-tRFs with lengths between 17-24 nt in normal breast (FIG. 8C).

Having decomposed the BRCA distribution into its normal (17 datasets) and tumor (294) components, the similarities and differences that might depend on tissue state were identified. The most striking differences were among the i′-tRFs and the 5′-tRFs, suggesting an intriguing and currently unexplored interconnection between the two categories of fragments.

As can be seen from FIG. 8, the proportion of internal fragments with 20 nt length was nearly halved in the tumor datasets compared to normal (p-val<10⁻³). The proportion of 5′-tRFs with 19 nt length and with lengths≧30 nt were more than doubled in the tumor datasets (p-val<10⁻³ for both comparisons). It appears as if the normal datasets preferentially produced i-tRFs while also reducing the expression of the 5′-tRFs, with a reversal of this situation occurring in the tumor. Notably, the relative abundance for the rest of the i-tRFs and 5′-tRFs remained largely unchanged between normal and tumor.

The tRNA Fragments have Relative Abundances that are Tissue-Specific and Tissue-State-Specific

In the context of messenger RNA (mRNA) expression studies, the abundance profiles of mRNAs that are common to two tissues can be used to tell the tissues apart (tissue-specific mRNA “signatures”). Similarly, for a given tissue, mRNA abundance profiles can distinguish between normal and disease states (tissue-state-specific mRNA “signatures”). It was determined whether tRFs possess similar properties.

To investigate the possibility of a tissue-specific profile, 200 tRFs common to the datasets were focused on: a) the subset of 253 female datasets from the LCL dataset (all of whom are healthy), and b) the 17 normal (female) datasets from the BRCA dataset.

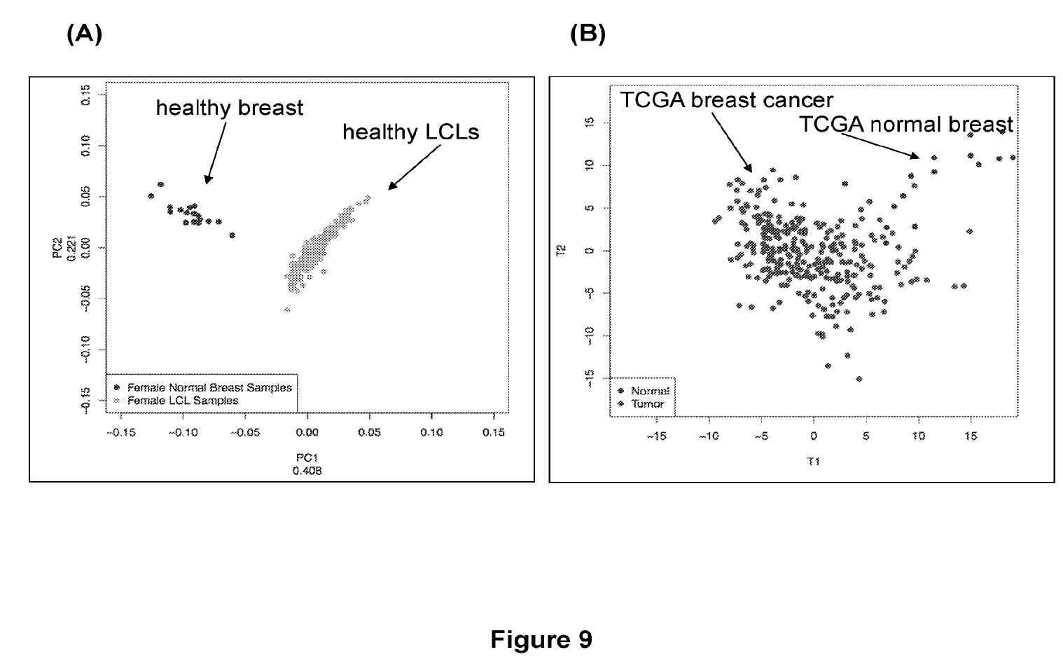

In FIG. 9A, a principal component analysis (unsupervised) of the abundances of the 200 fragments is shown to distinguish between the two tissues. It is important to note how characteristically tight each of the two point clusters is. This indicates that the abundance profiles of these 200 tRNA fragments were very similar across all datasets belonging to the same cluster. The within-group similarity of the tRF abundance profiles further supports the view that these fragments were constitutive in nature and not degradation products.

As the LCL and BRCA datasets come from two distinct studies, the possibility that the differences were due to biases caused by either the sequencing methods and/or by the whole experimental handling of the datasets needed to be excluded. Due to the lack of standard datasets that were common to both studies, the data was truncated by rank-normalizing the two datasets. By ranking the expression in each dataset, much of the quantitative information was lost and only the relative ordering based on abundance was retained.

By performing PCA on this truncated dataset, the two datasets were easily distinguished, which indicates that the differences in the abundance profiles were of a biological basis, not due to experimental biases. SAM, a non-parametric significance analysis method, was used to identify quantitative differences between the two datasets. Most of the fragments were differentially abundant between the two tissues. More than 30% of the significantly differentiated fragments were i-tRFs, which further argues for the importance of this novel category of tRFs.

To investigate the possibility of a tissue-state-specific profile, a single group was formed by combining all tumor datasets, independent of hormone status. Unlike the above example, this dataset has an artificially increased underlying heterogeneity, the result of having combined all breast cancer subtypes into a single group of datasets. A supervised clustering approach was used, namely PLS-DA. FIG. 9B shows that PLS-DA can easily distinguish between the two sets based on the abundance levels of these tRFs. It is also worth noting that the tumor dataset heterogeneity is reflected by the lack of tightness in the formed tumor cluster of FIG. 9B.

The tRNA Fragments Exhibit Race-Dependent Differences at the Tissue, Cellular, and Molecular Levels

In recent work, transcripts whose abundance differed across human races, between males and females, or between population groups was reported. Considering that both the LCL and the BRCA samples included individuals belonging to different races, it was determined whether the abundance profiles of the tRNA fragments exhibited any differences along this dimension.

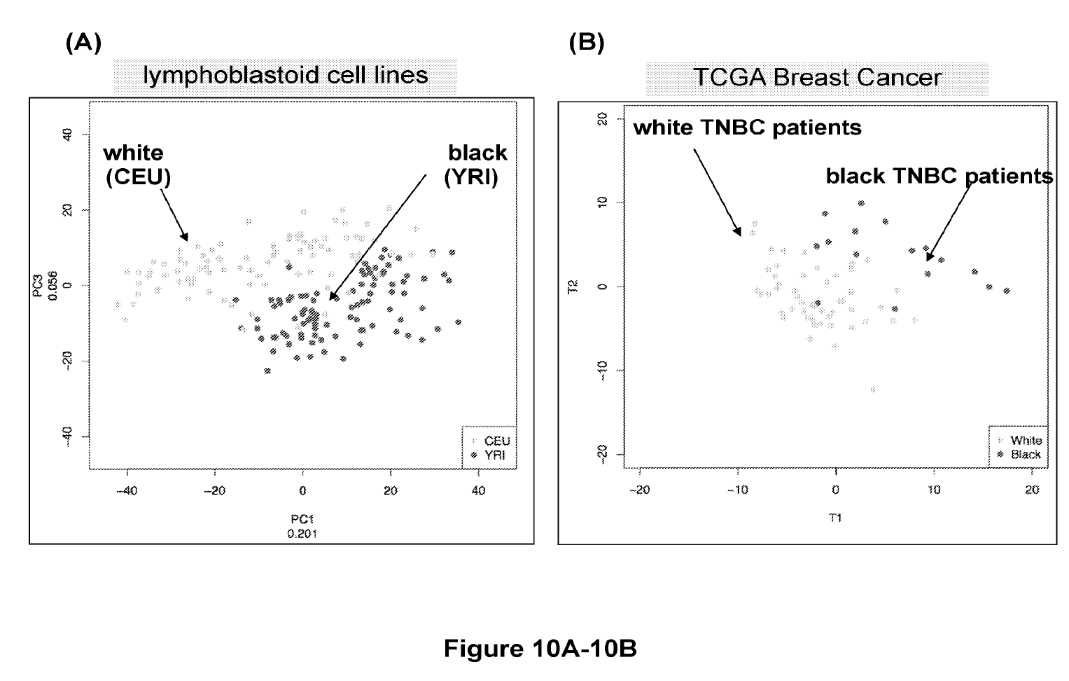

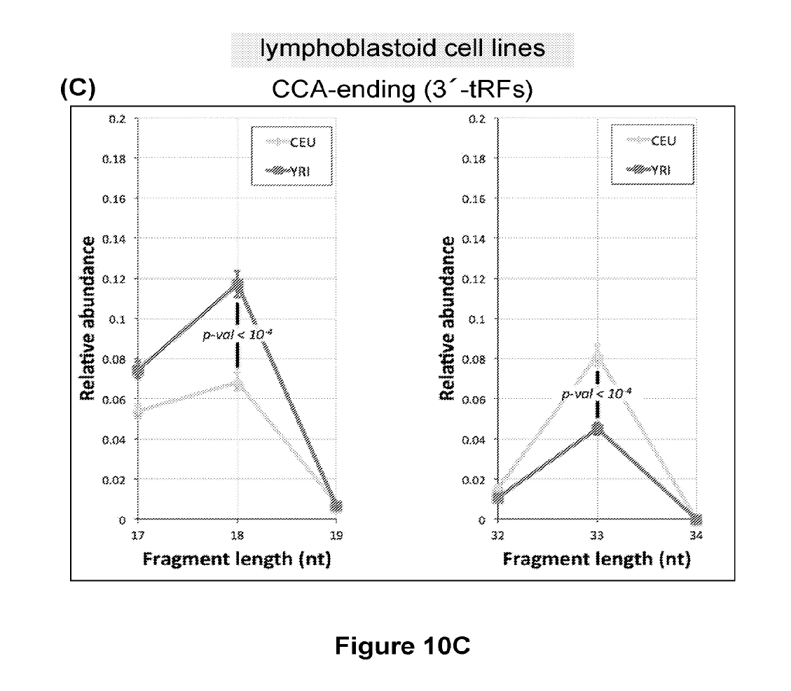

The transcriptional profiles in the LCL samples of the 93 samples originating from the CEU (white) group vs those of the 95 from the YRI (black) group were compared. FIG. 10A shows the results of the (unsupervised) Principal Component Analysis (PCA) for the CEU/YRI subset of the LCL dataset. The 1^{st} and 3^{rd} principal component provided a good separation of the two groups with modest cross-talk, indicating that the tRNA fragments exhibited race-dependent transcriptional differences at the cellular level (EBV-immortalized B-cells).

The subset of 78 triple negative breast cancer samples from the BRCA dataset were examined. This subset contained adequate numbers of black (16) and white (51) patients to permit statistical analyses. Because of the underlying heterogeneity of the analyzed cells, a supervised approach (Partial Least Squares-Discriminant Analysis, PLS-DA) was used. FIG. 10B shows the results: as with the LCL samples, there was an evident separation between the white and the black patients that was characterized by only modest cross-talk, indicating that the tRNA fragment profile differed between human races at the tissue level as well.

To investigate the possibility that differences existed among different populations, the graphs of FIGS. A-3D were decomposed into their constituent population components to determine if the curves of all five populations followed a similar pattern. However, a closer look allowed the identification of significant differences in the length distributions among races. FIG. 10C shows a detail for the CEU and YRI populations. As can be seen, there were nearly twice as many 18-mers among the fragments of the YRI population compared to the CEU population (p-val≦10⁻⁴). For fragments with a length of 33 nts, the situation was reversed with the CEU population making twice as many compared to the YRI (p-val≦10⁻⁴). In other words, even though the curves of the five populations were qualitatively similar, there were quantitative and statistically significant differences in the lengths and abundances of the fragments produced by members of the CEU and YRI groups at the molecular level as well.

In light of these observations, identification of which tRNA fragments had significantly different abundances between the two populations was determined. SAM, a nonparametric clustering method, was used. At a strict FDR of 0.00%, SAM identified 93 differentially expressed tRNA fragments: 48 had lower expression in the YRI samples compared to the CEU ones, whereas the remaining 45 had higher expression. Interestingly, the vast majority of the tRNA fragments with lower expression in the YRI group were of mitochondrial origin. Specifically, they were i-tRFs of the mitochondrial SerGCT tRNA that started around position +13 and ended around position +43. Mitochondrial tRNAs, ValTAC and PheGAA, also contributed significantly to the list of fragments that were differentially expressed between CEU and YRI.

Among the fragments that had higher expression in the YRI samples compared to the CEU ones and were identified by SAM, those originating from the LysCTT anticodon were dominant. Of the 45 tRNA fragments emerging from the template, 30 were statistically significant. An additional 5 statistically significant fragments in this category came from the LysTTT anticodon. The majority of the LysCTT fragments began before position +8 of the mature tRNA and ended just before the anticodon triplet (nucleotide 33 using trna13 of LysCTT on chromosome 14 as a reference). Only 2 of the 30 were classic 5′ tRNA halves (they start at position +1), whereas the rest were novel internal fragments. The 5′ terminus of these fragments was located between positions +1 and +7 inclusive and there was no consensus length: their length ranged between 21 and 33 nts.

The tRNA Fragments Exhibit Gender-Dependencies

The possibility that the tRNA fragments showed differences across gender boundaries was examined. Among the 452 LCL samples, men and women as well as the five populations (CEU, FIN, GBR, TSI, YRI) were evenly represented. There was a tendency for separation, but not a clear discrimination of the two genders. Specifically, the read length distributions of FIGS. 3A-3D were decomposed, but separately for men and women and for the five populations.

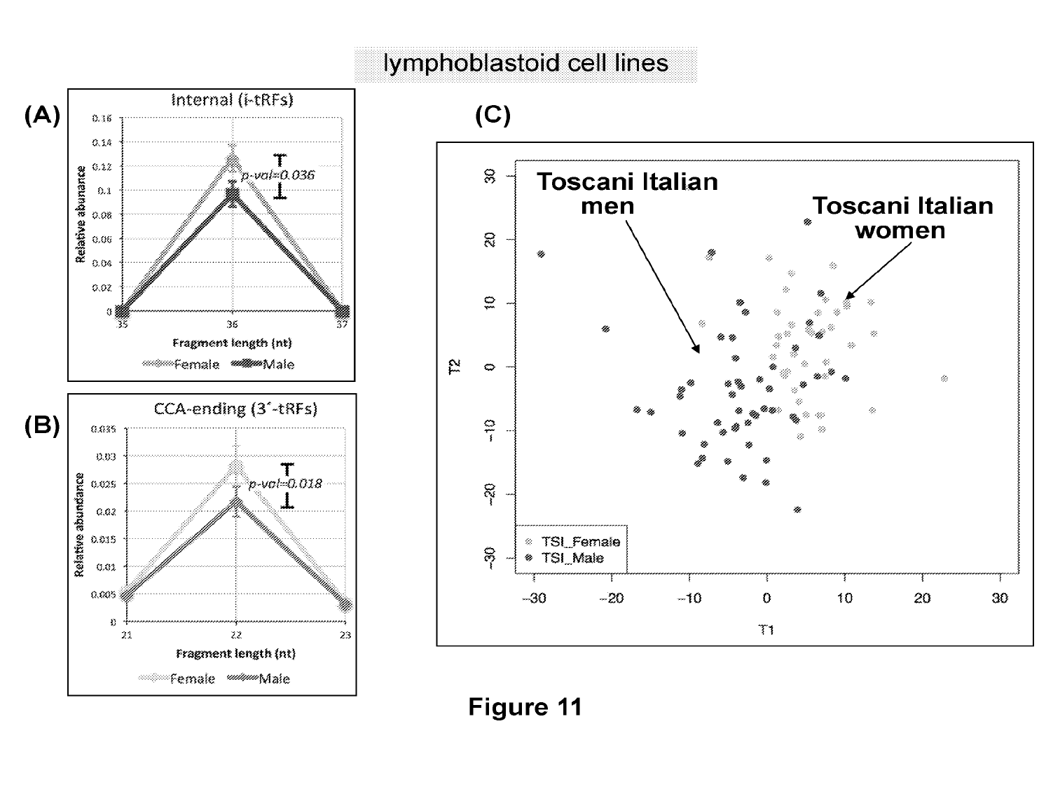

FIG. 11A shows the distributions for i-tRFs from men and women (YRI datasets only) for the internal 36-mers. These i-tRFs are less abundant in YRI males compared to YRI females (p-val=0.036). FIG. 11B shows analogously a portion of the distribution for CCA-ending fragments from men and women (TSI datasets only).

In the TSI population, these 22-mers were more abundant in women compared to men with the difference statistically significant (p-val=0.018). Using PLS-DA on the TSI men and women, a trend is seen for separation of the two genders (FIG. 11C). Among the fragments that are significant for the construction of the PLS-DA-driven separation (VIP scores>1.5), more than half (49 out of 94) are i-tRFs.

The tRNA Fragments Exhibit Abundances that Depend on Disease Subtype

The different tumor subtypes captured by the BRCA datasets were analyzed to investigate whether the profiles of tRFs differed between tumor subcategories. For this analysis, three subsets were used: the normal breast datasets, the ER−/PR−/HER2− (triple negative) datasets, and the ER+/PR+/HER2+(triple positive) datasets. Since the tRF profiles have been shown to be ethnicity-dependent, a single race was chosen, in particular white women who were represented in the BRCA collection at adequately high numbers (15 normal, 24 triple positive and 51 triple negative datasets).

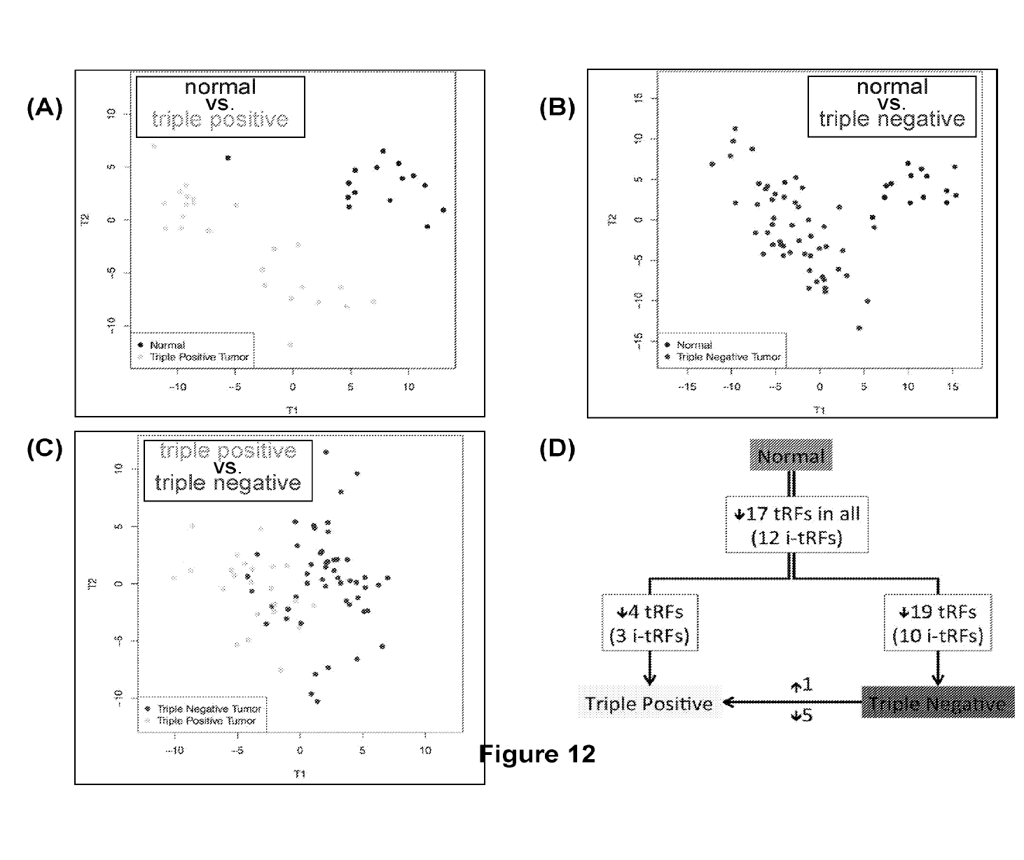

Pair-wise PLS-DA analyses were performed. In all three cases, the two categories being compared were distinguished clearly from one another (FIG. 12A-12C). Importantly, the ability to discriminate the two tumor subtypes based on tRNA fragment abundance suggests a potentially significant role for these fragments in the respective biology of breast cancer subtypes.

All of the statistically significant tRFs had lower abundance in the tumor datasets compared to the normal datasets (FIG. 12D). The findings were cross-validated through an independent SAM analysis. In concordance with the PLS-DA model, SAM also identified the same 17 fragments as having lower abundance in each tumor subtype compared to the normal datasets. Triple negative tumors were characterized by an additional 19 fragments with lower abundances in the tumor compared to the normal datasets (for a total of 36 fragments in the triple negative subtype).

It is important to note that the majority of differentially abundant tRFs in the two normal vs. tumor comparisons were from the internal region, i.e. i-tRFs (FIG. 12D). In the intra-tumor comparison, the differentially abundant tRFs were all 5′-tRFs and most of them were 19-mers from different genomic loci of the nuclear ArgTCG anticodon. These findings are in concordance with FIGS. 4A and 4B, validated by two independent statistical methods (PLS-DA and SAM), which in turn suggests the existence of concrete differences in the abundance of the tRNA fragment population in the two disease subtypes.

### TRFs are Loaded on Argonaute in a Cell-Line-Specific Manner

Previous work demonstrated that tRFs could be loaded on Argonaute (Ago), which indicates that one tRF function is through the RNAi pathway. No previous reports have examined differential Ago-loading of tRFs as a function of tissue, disease-state, ethnicity, or disease subtype.

To this end, publicly available Ago HITS-CLIP datasets were analyzed for three different breast cancer cell lines, each of which models specific breast cancer categories: MDA-MB-231 (ER−/PR−/HER2−), MCF7 (ER+), and BT-474 (HER2+). For consistency, and since the TCGA-BRCA dataset contained only reads≦30 nt, the HITS-CLIP datasets were analyzed using only sequenced reads that were ≦30 nt long.

70 of the abundant fragments originated in the internal (i-tRFs) and 68 in the CCA-ending (3′-tRFs) regions. By comparison, only 25 abundant 5′-tRFs were loaded on Argonaute.

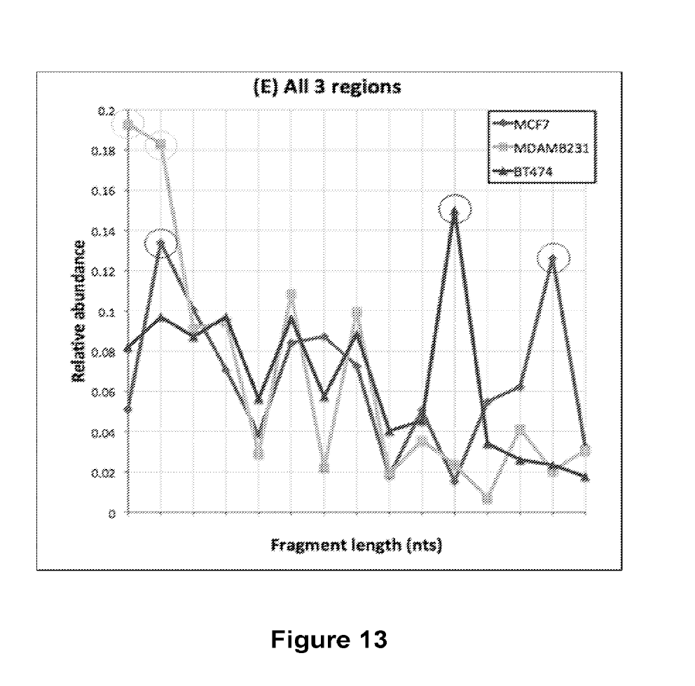

The length distributions of all Ago-loaded tRFs with length≦30 nt were analyzed in the three cell lines. Interestingly, each cell line was found to have its own distinct profile of Ago-loaded fragments (FIG. 13). In particular, BT-474 cells exhibited a peak for 26-mers that mainly included i-tRFs. On the other hand, MDA-MB-231 had a prevalence for Ago-loaded 16-mers, 17-mers, and 21-mers 3′-tRFs and 23-mers from the internal origin (i-tRFs). MCF-7 cells exhibited a prevalence for Ago-loaded 17-mers and 29-mers. These findings support a model where the tRNA fragments are preferentially Ago-loaded in a manner that is cell-line-specific, presumably reflecting disease-subtype specificity. Also, the results further corroborate the functional roles for the shorter tRFs through their participation in the RNAi pathway as miRNA-like entities.

Fragment-Specific PCR-Based Validation of Internal tRNA Fragments in Clinical Samples and Cell Lines

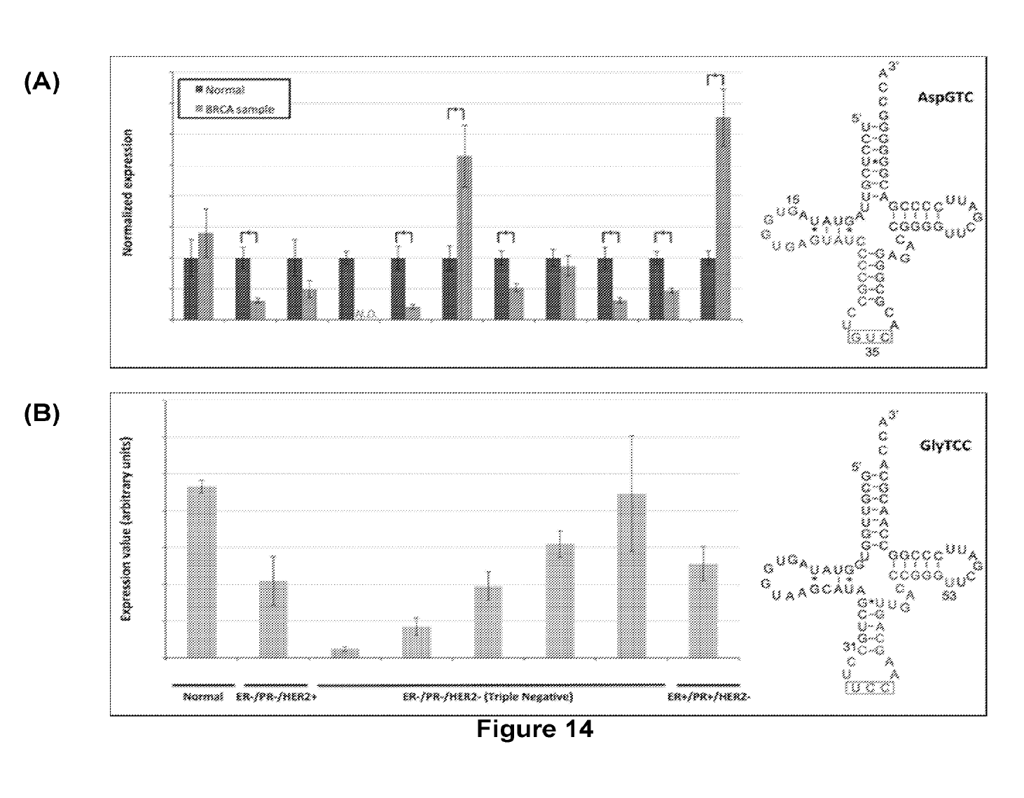

The tRFs that arise from the internal region of mature tRNAs represent a novel category of tRFs. Independent experimental validation was sought for these novel molecules. For this purpose, one i-tRF was selected that begins within the loop region of the D-loop of AspGTC and ends at the anticodon (FIG. 14A) and one that starts just before the anticodon loop and ends at the T-loop of GlyTCC (FIG. 14B). Both fragments were identified repeatedly in the analyses of the BRCA datasets. The quantification task challenging is due to the requirement that the fragment must be amplified while ensuring that the amplified molecule has the same endpoints captured by the RNA-seq datasets. To this end, the “dumbbell-PCR” was used to detect RNA molecules with a specified length and specified endpoints.

The FIREPLEX® (Firefly BioWorks, Boston, Mass.) approach, a method with single nucleotide specificity, for quantification of the second tRF, was also used. Total RNA extracted from 11 breast tumor and 11 adjacent normal breast samples was used for starting material. Quantification of the AspGTC tRNA fragment and total RNA from eight different normal or breast cancer cell lines, as well as quantification of the GlyTCC-derived fragment, was performed.

The tRF from the AspGTC anticodon was specifically amplified and its expression was quantified in 21 of the 22 experiments (FIG. 14A). In five of the 11 analyzed pairs, there was a statistically significant decrease in the tumor sample (p-val<0.01; Student's t-test). In two other samples, the fragment's expression was statistically significantly increased in the tumor (p-val<0.01; Student's t-test).

These results validate the existence of the novel i-tRFs in independent samples and provide initial evidence that such fragments have differential abundancies in healthy and breast tumor tissue. The results further agree with the analysis of the BRCA datasets. The second i-tRF, from the GlyTCC tRNA, spanning the anticodon triplet was quantified in eight different normal and breast cancer cell lines using the multiplex miRNA assay, which is based on the FIREPLEX® approach (FIG. 14B). In all of the cases, the i-tRF was detected and present at significantly increased levels over the background threshold.

A Single Locus can Give Rise to Many tRNA Fragments in a Single Tissue

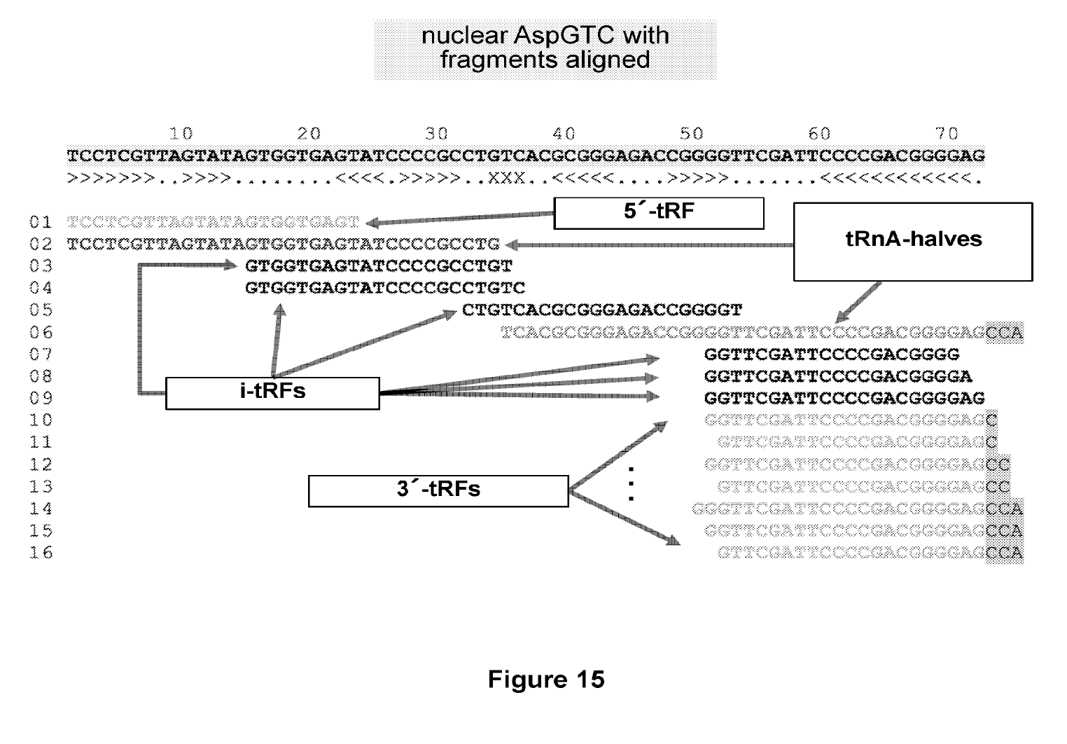

In the analysis of the breast samples from the TCGA repository, many short RNAs were identified that were statistically significant, and present in the samples of multiple individuals. Even though the specifics may have differed slightly from one isodecoder to the next (e.g. nuclear trna78-AspGTC vs. nuclear trna144-AspGTC), the basic behavior for instances of the same tRNA anticodon remained the same. For example, nuclear AspGTC gave rise to diverse fragments (FIG. 15).

Targeting by tRFs

Loading of tRNA fragments on Argonaute indicates that they act as miRNA-like guide-RNAs for Ago and possibly participate in the RNA interference pathway. The targets of these miRNA-like tRNA fragments are referred to as “interlocked” targets. In addition, others have published work where it was shown that a transcript A can “target” and modulate the abundance of another transcript B simply by acting as a molecular decoy for a miRNA or an RNA binding protein (RBP) that would otherwise interact with B. In this case, B is a “decoyed” target of A (and vice versa). Clearly, both modes of targeting are of interest in the tRNA fragment setting. To this end, two algorithms were devised: one for predicting interlocked targets and one for predicting decoyed targets.

### Algorithm for Predicting Interlocked Targets

The methodology used to design rna22, a very popular miRNA target prediction algorithm, was used. Briefly: a) a list was made of the sequences of all tRFs that were statistically significant across all analyzed datasets for the cancer being studied; b) the sequences were analyzed with Teiresias, a publicly available pattern discovery algorithm, to identify salient sequence features that were shared by two or more tRFs—the similarity in the isodecoder sequences guaranteed that such patterns do exist; c) the patterns were reverse-complemented and populated with a hash-table; d) the hash-table was used to process the transcripts of all mRNAs and ncRNAs whose abundance was above the threshold in the long RNA-seq datasets of the cancer's samples. A target site contained in an mRNA or ncRNA led to a pattern accumulation at that site and the formation of a “bump;” e) using a threshold obtained through a Monte Carlo simulation with random strings, the bumps with low heights were filtered out (support by only a few patterns); f) for each putative target site, the RNAfold or a similar algorithm was used to generate patterns of each tRFs to form the final candidate tRF:target heteroduplexes. Table 1 shows an example target.

TABLE 1
Examples of predicted tRF mRNA interactions from
the TCGA BRCA analyses.

| | |
|---|---|
| A) | RAB34:AspGTC tRF [positions 31-53] |
| | 5′→ATTCT--TCTTCCGTGTGGCAGC→3′ |
| | \|::\|: \|\|\|:\|\|\|:\|\|\|:\|\|\|\| |
| | 3′←TGGGGCCAGAGGGCGCACTGTCC←5′ |
| B) | SIKE1:GLnTTG tRF [positions 1-30] |
| | 5′→GGCCCCATTGTGTAATAGTTAGCACTCTAA→3′ |
| | \|\| \|\|\|\|\| \|\|\|\|\|\|\| \|\|\|\|\|\|\|\|\|\|\| |
| | 5′→GGTCCCATGGTGTAATGGTTAGCACTCTGG→3′ |

A) an interlocked target prediction (RAB34).
B) a decoyed target prediction (SIKE1).

### Algorithm for Predicting Decoyed Targets.

For this, the steps of the interlocked-target algorithm were followed with the following key modifications: i) in step c) the patterns were not reverse-complemented prior to populating the hash-table; ii) in step f) any of a number of standard algorithms were used, such as BLAST, FASTA etc., to search the patterns generated by each of the tRFs to see which matched the target site the best (=best local alignment). Unlike the previous algorithm, in this algorithm a “bump” indicated that the mRNA or ncRNA sequence fragment under it resembled a similarly-sized segment among the tRFs at hand. Table 1 shows one such example target.

The Materials and Methods used in the performance of the experiments disclosed herein, which have not been covered already, are now described.

Notation

To facilitate the discussion, the notation that is used by tRNAscan-SE was augmented. In particular, the existing labels were tagged with fragment-specific information, namely the relative positions inside a reference tRNA and the number of appearances in other tRNAs of the same or different anticodons. For example, the augmented label

trna116_GluCTC_1_-_14539923_1453999304@23.45.23_1_0_8 refers to the tRNA fragment that has length of 23 and spans positions 23 through 45 inclusive of the mature trna116 of GluCTC. The latter being located on the reverse (negative) strand of chromosome 1 between positions 145399233 and 145399304 inclusive. In the cases where more than one genomic tRNA loci produces the fragment, only one tRNA locus was chosen to serve as a source-proxy.

The last three numbers of the augmented label that follow the double underscore captured the following information: a) the number of different anticodons that may give rise to this fragment (1 in the above example), the number of pseudo tRNAs that also contain this fragment sequence (0 in the example), and the total number of genomic loci within the tRNA space (see below) that are possible sources of the fragment (8 in this case). Lastly, for fragments whose 3′ end is within the span of the terminal CCA, the infix “CCA” was added before the double underscore, e.g., trna75_MetCAT_6_+_28912352_28912424@57.76.20.CCA_1_0_2.

Defining the tRNA Space

Directly linked to mapping is the definition of what constitutes the genome's tRNA space. For the purposes described herein, the following were combined:

a) the 22 known human mitochondrial tRNA sequences (NCBI entry NC_012920.1);

b) 610 (508 true tRNAs and 102 pseudo-tRNAs) of the 625 nuclear tRNA sequences from gtRNAdb. The selenocysteine tRNAs were excluded from the considered gtRNAdb entries, tRNAs with undetermined anticodon identity, and tRNAs mapping to contigs that were not part of the human chromosome assembly;

c) the eight genomic intervals chr1:+:566062-566129, chr1:+:568843-568912, chr1:−:564879-564950, chr1:−:566137-566205, chr14:+:32954252-32954320, chr1:−: 566207-566279, chr1:−:567997-568065, and, chr5:−:93905172-93905240—all coordinates were from the hg19 assembly of the human genome—that corresponded to identical instances of seven mitochondrial tRNAs TrpTCA, LysTTT, GlnTTG, AlaTGC (×2), AsnGTT, SerTGA, and, GluTTC respectively.

In total, the tRNA space included 640 sequences.

Mapping on the Genome

The repeating nature of tRNA sequences required that special steps be taken when mapping the RNA-seq data on the genome.

i) Multiple hits: To account for any given tRNA anticodon having multiple genomic locations and properly mapping the sequenced reads arising from such loci, any given sequenced read was permitted to potentially map up to 10,000 distinct genomic locations.

ii) Exact matches: To accommodate the possibility of occasional errors manifested in the form of nucleotide replacements, nucleotide insertions or deletions (indels), or various combinations thereof, a small number of indels and mismatches was permitted during the mapping step of the deep sequencing. More flexibility and improved mapping rates translated into localization errors, when working with tRNAs. Therefore, a conservative mapping strategy and exact mapping of reads on the genome was employed to map without any insertions or deletions.

iii) Mapping the full genome: As disclosed herein, compiling a database of all known tRNA sequences and then mapping the sequenced reads would miss the fact that some segments of the known tRNAs also appear inside non-tRNA sequences, and lead to incorrect conclusions. Mapping the sequenced reads on the full genome then post-processing each mapped read and discarding those that map both inside and outside the known tRNAs.

iv) Presence of terminal CCA: Any sequenced reads that corresponded to the 3′ of mature tRNAs included the post-transcriptionally added terminal triplet CCA. Exact mapping of the reads did not accommodate CCA's presence. Instead, prior to mapping, a modified instance of the genome was created where CCA was used to replace the three genomic nucleotides immediately downstream of each of the 640 reference mature tRNAs.

### Other Embodiments

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of identifying a subject in need of therapeutic intervention to treat a disease or condition, disease recurrence, or disease progression comprising:
isolating fragments of tRNAs from a sample obtained from the subject; and
characterizing the tRNA fragments and their relative abundance in the sample to identify a signature, wherein when the signature is indicative of a diagnosis of the disease treatment of the subject is recommended.

2. The method of claim 1, wherein the sample is isolated from a cell, tissue or body fluid obtained from the subject.

3. The method of claim 2, wherein the body fluid is selected from the group consisting of amniotic fluid, aqueous humour and vitreous humour, bile, blood serum, breast milk, cerebrospinal fluid, cerumen, chyle, chyme, endolymph and perilymph, exudates, feces, female ejaculate, gastric acid, gastric juice, lymph, mucus, pericardial fluid, peritoneal fluid, pleural fluid, pus, rheum, saliva, sebum, serous fluid, semen, smegma, sputum, synovial fluid, sweat, tears, urine, vaginal secretion, and vomit.

4. The method of claim 1, wherein the sample is selected from the group consisting of a peripheral blood cell, a tumor cell, a circulating tumor cell, an exosome, a bone marrow cell, a breast cell, a lung cell, and a pancreatic cell.

5. The method of claim 1, wherein the tRNA fragments are isolated by a method selected from the group consisting of size selection, sequencing, and amplification.

6. The method of claim 1, wherein isolating the tRNA fragments comprises isolating tRNA fragments with a length in the range of about 15 nucleotides to about 80 nucleotides.

7. The method of claim 1, wherein isolating the tRNA fragments comprises isolating tRNA fragments having a predominant length of 16, 17, 26, or 29 nucleotides is indicative of a breast cancer subtype.

8. The method of claim 1, wherein the signature is obtained through sequence-specific methods that preserve at least one terminus of the tRNA fragments.

9. The method of claim 1, wherein the signature is obtained by hybridization to a panel of oligonucleotides.

10. The method of claim 9, wherein the tRNA fragments are enriched prior to the hybridization.

11. The method of claim 9, wherein the oligonucleotide panel comprises at least two or more polynucleotides that selectively hybridize to the tRNA fragments.

12. The method of claim 1, wherein the signature comprises at least one sequence with identifiers selected from the group consisting of SEQ ID NOs: 1-1802 to analyze brain; SEQ ID NOs: 8538-8852 to analzye breast tissue; SEQ ID NOs: 12462-14475 to analyze blood cells; SEQ ID NOs: 24833-25945 to analyze blood cells; SEQ ID NOs: 36100-37466 to analyze pancreatic cancer; SEQ ID NOs: 42349-43721 to analyze prostate tissue; and SEQ ID NOs: 51286-51793 to analyze platelets.

13. The method of claim 1, wherein the signature comprises at least one sequence with identifiers selected from the group consisting of SEQ ID NOs: 11, 18, 19, 28, 31, 34, 43, 51, 59, 83, 189, 194, 209, 268, 305, 306, 307, 316, 320, 398, 404, 611, 632, 653, 696, 751, 768, 816, 817, 860, 869, 870, 871, 920, 921, 925, 951, 960, 967, 989, 1005, 1030, 1133, 1201, 1202, 1223, 1229, 1230, 1231, 1240, 1248, 1298, 1318, 1406, 1412, 1421, 1425, 1453, 1510, 1577, 1582, 1631, 1637, 1645, 1661, 1695, 1727 and 1794 to distinguish Alzheimer's disease brain from normal brain.

14. The method of claim 1, wherein the signature comprises at least one sequence with identifiers selected from the group consisting of SEQ ID NO:8613 and SEQ ID NO: 8823 to distinguish triple negative breast cancer from HER2+ breast cancer.

15. The method of claim 1, wherein the signature comprises at least one sequence with identifiers selected from the group consisting of SEQ ID NOs: 8542, 8543, 8566, 8579, 8582, 8587, 8589, 8590, 8594, 8671-8673, 8707, 8731, 8774-8778, 8803, 8827-8828, 8831-8832, 8837-8838, and 8852 to distinguish triple negative breast cancer from normal.

16. The method of claim 1, wherein the signature comprises at least one sequence with identifiers selected from the group consisting of SEQ ID NOs: 8596, 8601, 8622, 8657, 8664, and 8811 to distinguish triple positive breast cancer from triple negative breast cancer.

17. The method of claim 1, wherein the signature comprises at least one sequence with identifiers selected from the group consisting of SEQ ID NOs: 8582, 8599-8601, 8622-8623, 8634, 8657, 8663-8665, 8676, 8698, 8703-8706, 8718-8720, 8722, 8724, 8738, 8745, 8758, 8761, 8767-8772, and 8840 to distinguish breast cancer from normal tissue.

18. The method of claim 1, wherein the signature comprises at least one sequence with identifiers selected from the group consisting of SEQ ID NOs: 12462, 12463, 12464, 12465, 12466, 12467, 12468, 12469, 12470, 12471, 12472, 12473, 12474, 12475, 12476, 12477, 12478, 12479, 12480, 12481, 12482, 12483, 12484, 12485, 12486, 12487, 12488, 12489, 12490, 12492, 12493, 12494, 12495, 12496, 12497, 12498, 12499, 12500, 12501, 12502, 12503, 12504, 12505, 12506, 12507, 12508, 12509, 12510, 12511, 12512, 12513, 12514, 12515, 12516, 12517, 12518, 12519, 12520, 12522, 12523, 12524, 12525, 12526, 12527, 12529, 12530, 12531, 12532, 12533, 12534, 12536, 12537, 12538, 12540, 12541, 12542, 12543, 12544, 12545, 12546, 12547, 12548, 12549, 12550, 12551, 12552, 12553, 12554, 12555, 12556, 12557, 12558, 12559, 12560, 12561, 12562, 12563, 12564, 12565, 12566, 12567, 12568, 12569, 12570, 12572, 12573, 12574, 12575, 12576, 12577, 12578, 12580, 12581, 12582, 12584, 12585, 12586, 12587, 12588, 12589, 12590, 12591, 12592, 12593, 12594, 12595, 12596, 12597, 12598, 12599, 12600, 12601, 12602, 12603, 12604, 12607, 12608, 12609, 12614, 12615, 12616, 12617, 12618, 12619, 12620, 12621, 12622, 12623, 12624, 12625, 12626, 12627, 12628, 12629, 12631, 12632, 12633, 12634, 12635, 12636, 12637, 12638, 12639, 12640, 12641, 12642, 12643, 12645, 12647, 12648, 12649, 12652, 12653, 12654, 12655, 12657, 12658, 12659, 12660, 12661, 12663, 12664, 12665, 12666, 12667, 12668, 12669, 12670, 12671, 12672, 12674, 12677, 12678, 12679, 12680, 12682, 12684, 12685, 12686, 12687, 12688, 12689, 12690, 12691, 12692, 12693, 12694, 12695, 12696, 12697, 12698, 12699, 12700, 12703, 12704, 12705, 12706, 12708, 12710, 12711, 12712, 12713, 12714, 12715, 12716, 12717, 12718, 12719, 12720, 12721, 12724, 12726, 12727, 12728, 12729, 12730, 12731, 12732, 12733, 12734, 12736, 12738, 12739, 12740, 12741, 12742, 12743, 12744, 12745, 12746, 12747, 12749, 12750, 12751, 12754, 12756, 12758, 12760, 12761, 12763, 12764, 12765, 12766, 12767, 12768, 12769, 12770, 12771, 12773, 12774, 12776, 12777, 12779, 12780, 12781, 12782, 12783, 12785, 12786, 12788, 12789, 12790, 12791, 12792, 12795, 12799, 12800, 12801, 12802, 12803, 12804, 12805, 12806, 12807, 12809, 12811, 12812, 12813, 12814, 12815, 12817, 12818, 12819, 12820, 12821, 12824, 12825, 12826, 12827, 12828, 12829, 12831, 12832, 12833, 12834, 12835, 12836, 12837, 12838, 12840, 12841, 12842, 12843, 12844, 12846, 12847, 12848, 12849, 12850, 12851, 12852, 12853, 12854, 12855, 12856, 12857, 12858, 12859, 12860, 12861, 12864, 12865, 12867, 12868, 12869, 12870, 12871, 12872, 12873, 12874, 12875, 12876, 12877, 12878, 12879, 12880, 12881, 12882, 12883, 12884, 12885, 12886, 12887, 12888, 12889, 12890, 12891, 12892, 12893, 12894, 12895, 12896, 12897, 12899, 12900, 12901, 12902, 12903, 12904, 12905, 12906, 12907, 12909, 12910, 12911, 12912, 12913, 12914, 12916, 12918, 12919, 12920, 12922, 12923, 12924, 12925, 12926, 12927, 12928, 12929, 12930, 12931, 12932, 12933, 12934, 12935, 12936, 12937, 12938, 12939, 12940, 12941, 12942, 12943, 12944, 12946, 12947, 12948, 12949, 12950, 12951, 12954, 12955, 12956, 12957, 12958, 12959, 12960, 12961, 12962, 12963, 12965, 12966, 12967, 12968, 12969, 12970, 12971, 12972, 12973, 12974, 12975, 12978, 12979, 12980, 12981, 12982, 12983, 12984, 12985, 12986, 12987, 12988, 12990, 12991, 12992, 12993, 12994, 12996, 12997, 12998, 12999, 13000, 13001, 13002, 13003, 13004, 13005, 13006, 13007, 13008, 13009, 13011, 13012, 13013, 13014, 13016, 13017, 13018, 13019, 13020, 13021, 13022, 13023, 13024, 13025, 13028, 13029, 13030, 13031, 13033, 13034, 13035, 13036, 13037, 13038, 13039, 13040, 13044, 13045, 13046, 13047, 13049, 13050, 13051, 13052, 13053, 13054, 13055, 13056, 13057, 13058, 13059, 13061, 13063, 13065, 13066, 13067, 13068, 13069, 13070, 13071, 13072, 13073, 13074, 13075, 13076, 13077, 13078, 13079, 13080, 13081, 13082, 13083, 13084, 13085, 13086, 13087, 13088, 13089, 13090, 13091, 13092, 13093, 13094, 13095, 13096, 13097, 13098, 13100, 13101, 13102, 13103, 13104, 13105, 13106, 13107, 13110, 13112, 13113, 13114, 13117, 13118, 13119, 13120, 13121, 13122, 13123, 13124, 13125, 13127, 13128, 13129, 13130, 13131, 13132, 13133, 13134, 13135, 13136, 13137, 13138, 13139, 13140, 13141, 13142, 13143, 13145, 13146, 13148, 13149, 13150, 13151, 13152, 13153, 13154, 13155, 13157, 13158, 13159, 13160, 13161, 13162, 13163, 13164, 13165, 13166, 13167, 13168, 13169, 13170, 13171, 13174, 13175, 13177, 13178, 13179, 13181, 13182, 13183, 13184, 13185, 13186, 13187, 13189, 13190, 13191, 13193, 13195, 13196, 13198, 13199, 13200, 13201, 13202, 13203, 13204, 13205, 13206, 13207, 13208, 13209, 13210, 13211, 13212, 13213, 13214, 13215, 13216, 13217, 13218, 13219, 13221, 13222, 13223, 13225, 13228, 13230, 13231, 13232, 13233, 13234, 13236, 13237, 13238, 13239, 13240, 13241, 13242, 13243, 13245, 13246, 13247, 13248, 13249, 13250, 13251, 13252, 13253, 13255, 13256, 13257, 13258, 13259, 13260, 13261, 13262, 13263, 13264, 13268, 13269, 13270, 13271, 13273, 13274, 13275, 13276, 13277, 13278, 13279, 13280, 13281, 13283, 13285, 13286, 13287, 13288, 13289, 13290, 13292, 13293, 13294, 13295, 13296, 13297, 13298, 13299, 13300, 13301, 13302, 13303, 13304, 13306, 13309, 13310, 13312, 13313, 13314, 13315, 13316, 13317, 13318, 13319, 13320, 13323, 13324, 13325, 13326, 13327, 13328, 13329, 13330, 13331, 13332, 13333, 13334, 13335, 13336, 13337, 13338, 13339, 13340, 13341, 13342, 13343, 13345, 13346, 13347, 13348, 13349, 13350, 13351, 13352, 13353, 13354, 13355, 13357, 13358, 13359, 13360, 13361, 13362, 13363, 13364, 13365, 13366, 13367, 13369, 13370, 13371, 13372, 13373, 13374, 13375, 13376, 13377, 13378, 13379, 13380, 13381, 13382, 13383, 13384, 13385, 13386, 13387, 13388, 13389, 13390, 13391, 13392, 13393, 13394, 13395, 13396, 13397, 13398, 13399, 13400, 13401, 13402, 13403, 13404, 13405, 13406, 13407, 13408, 13409, 13410, 13411, 13412, 13413, 13414, 13415, 13416, 13417, 13421, 13422, 13424, 13426, 13427, 13428, 13429, 13430, 13431, 13432, 13433, 13434, 13436, 13437, 13438, 13439, 13440, 13441, 13442, 13443, 13445, 13446, 13447, 13448, 13449, 13450, 13452, 13453, 13454, 13455, 13456, 13457, 13458, 13459, 13460, 13461, 13462, 13463, 13464, 13465, 13466, 13467, 13468, 13469, 13470, 13471, 13472, 13473, 13474, 13475, 13476, 13477, 13478, 13479, 13480, 13481, 13482, 13484, 13485, 13486, 13488, 13489, 13491, 13492, 13493, 13494, 13495, 13496, 13498, 13500, 13501, 13503, 13504, 13505, 13506, 13507, 13508, 13509, 13510, 13511, 13512, 13513, 13514, 13516, 13517, 13519, 13520, 13522, 13523, 13524, 13525, 13528, 13529, 13530, 13531, 13532, 13533, 13534, 13535, 13536, 13537, 13538, 13539, 13540, 13541, 13542, 13543, 13544, 13545, 13546, 13547, 13548, 13550, 13551, 13552, 13553, 13554, 13556, 13557, 13558, 13559, 13560, 13561, 13562, 13563, 13567, 13568, 13569, 13570, 13571, 13572, 13573, 13574, 13576, 13577, 13578, 13579, 13580, 13581, 13582, 13583, 13584, 13585, 13586, 13587, 13588, 13589, 13590, 13591, 13592, 13593, 13594, 13595, 13596, 13597, 13598, 13599, 13600, 13601, 13602, 13603, 13604, 13605, 13606, 13607, 13608, 13609, 13610, 13611, 13612, 13613, 13614, 13615, 13616, 13617, 13619, 13620, 13621, 13622, 13623, 13624, 13626, 13627, 13628, 13629, 13632, 13633, 13634, 13635, 13636, 13637, 13638, 13639, 13640, 13641, 13642, 13643, 13644, 13645, 13646, 13647, 13648, 13649, 13650, 13651, 13654, 13655, 13656, 13657, 13658, 13659, 13660, 13661, 13662, 13663, 13664, 13665, 13666, 13667, 13668, 13669, 13670, 13671, 13672, 13673, 13674, 13675, 13676, 13677, 13678, 13679, 13680, 13681, 13682, 13683, 13684, 13685, 13687, 13688, 13690, 13691, 13693, 13695, 13696, 13697, 13699, 13700, 13702, 13703, 13704, 13706, 13707, 13708, 13709, 13710, 13711, 13712, 13713, 13714, 13716, 13717, 13718, 13719, 13720, 13721, 13722, 13723, 13724, 13725, 13726, 13727, 13728, 13729, 13730, 13731, 13732, 13733, 13734, 13735, 13737, 13738, 13739, 13740, 13741, 13742, 13743, 13744, 13745, 13746, 13747, 13748, 13749, 13750, 13751, 13752, 13754, 13755, 13756, 13757, 13758, 13759, 13760, 13762, 13763, 13764, 13765, 13766, 13767, 13768, 13769, 13770, 13771, 13772, 13774, 13775, 13776, 13777, 13778, 13779, 13780, 13781, 13782, 13783, 13784, 13785, 13786, 13787, 13788, 13789, 13790, 13792, 13793, 13794, 13795, 13796, 13799, 13801, 13802, 13803, 13804, 13806, 13807, 13808, 13809, 13810, 13811, 13812, 13813, 13815, 13816, 13817, 13818, 13819, 13820, 13821, 13822, 13823, 13824, 13825, 13826, 13827, 13828, 13829, 13830, 13831, 13833, 13834, 13835, 13836, 13837, 13838, 13839, 13841, 13842, 13843, 13844, 13845, 13846, 13849, 13850, 13851, 13852, 13853, 13854, 13855, 13856, 13857, 13858, 13859, 13860, 13861, 13862, 13863, 13864, 13865, 13866, 13868, 13869, 13870, 13871, 13873, 13874, 13875, 13876, 13878, 13879, 13880, 13881, 13882, 13884, 13885, 13887, 13888, 13889, 13890, 13893, 13895, 13896, 13897, 13898, 13899, 13900, 13901, 13902, 13903, 13904, 13905, 13906, 13908, 13909, 13910, 13911, 13912, 13914, 13915, 13916, 13917, 13919, 13920, 13921, 13922, 13923, 13924, 13925, 13926, 13928, 13929, 13930, 13931, 13932, 13933, 13934, 13935, 13936, 13937, 13938, 13939, 13940, 13941, 13942, 13944, 13945, 13946, 13948, 13950, 13952, 13953, 13954, 13955, 13956, 13960, 13961, 13962, 13963, 13964, 13965, 13966, 13967, 13968, 13970, 13971, 13972, 13973, 13974, 13975, 13976, 13977, 13978, 13979, 13980, 13982, 13983, 13984, 13985, 13986, 13987, 13988, 13989, 13990, 13991, 13992, 13993, 13994, 13995, 13996, 13997, 13998, 13999, 14000, 14001, 14002, 14003, 14004, 14005, 14006, 14007, 14008, 14010, 14011, 14012, 14013, 14014, 14015, 14016, 14017, 14018, 14019, 14020, 14021, 14022, 14023, 14024, 14025, 14026, 14027, 14028, 14030, 14031, 14032, 14034, 14035, 14037, 14038, 14039, 14040, 14041, 14042, 14043, 14044, 14045, 14046, 14047, 14048, 14049, 14050, 14051, 14052, 14053, 14055, 14059, 14060, 14061, 14062, 14064, 14065, 14067, 14068, 14069, 14070, 14071, 14072, 14073, 14074, 14075, 14076, 14077, 14078, 14079, 14080, 14082, 14084, 14085, 14086, 14088, 14089, 14090, 14092, 14093, 14095, 14096, 14097, 14098, 14099, 14100, 14103, 14104, 14105, 14108, 14109, 14110, 14111, 14112, 14113, 14116, 14117, 14118, 14119, 14121, 14122, 14123, 14124, 14125, 14126, 14127, 14128, 14129, 14130, 14131, 14132, 14133, 14135, 14136, 14137, 14139, 14141, 14142, 14143, 14144, 14145, 14146, 14147, 14148, 14151, 14152, 14153, 14154, 14155, 14156, 14157, 14158, 14159, 14160, 14161, 14162, 14163, 14166, 14167, 14168, 14169, 14170, 14171, 14172, 14173, 14175, 14176, 14177, 14178, 14179, 14180, 14181, 14182, 14183, 14185, 14186, 14187, 14188, 14190, 14191, 14192, 14193, 14194, 14195, 14197, 14198, 14199, 14201, 14204, 14205, 14207, 14208, 14212, 14213, 14215, 14216, 14217, 14218, 14219, 14222, 14223, 14224, 14225, 14226, 14227, 14228, 14229, 14230, 14231, 14232, 14233, 14234, 14235, 14236, 14237, 14238, 14239, 14240, 14241, 14242, 14243, 14244, 14245, 14246, 14247, 14248, 14249, 14250, 14251, 14252, 14253, 14254, 14255, 14256, 14257, 14258, 14259, 14260, 14261, 14262, 14263, 14265, 14266, 14267, 14268, 14271, 14273, 14274, 14276, 14280, 14281, 14282, 14283, 14284, 14285, 14287, 14288, 14290, 14292, 14293, 14294, 14295, 14296, 14297, 14298, 14299, 14300, 14301, 14302, 14303, 14304, 14305, 14306, 14307, 14308, 14309, 14310, 14311, 14313, 14314, 14315, 14316, 14317, 14320, 14321, 14322, 14323, 14324, 14325, 14326, 14328, 14329, 14330, 14331, 14332, 14333, 14334, 14335, 14336, 14338, 14339, 14340, 14342, 14343, 14344, 14346, 14347, 14348, 14349, 14350, 14351, 14353, 14354, 14355, 14356, 14357, 14358, 14359, 14360, 14361, 14363, 14365, 14366, 14367, 14368, 14369, 14370, 14371, 14372, 14373, 14374, 14375, 14376, 14377, 14378, 14379, 14380, 14382, 14383, 14384, 14385, 14386, 14389, 14390, 14391, 14392, 14393, 14394, 14395, 14396, 14397, 14399, 14400, 14401, 14402, 14403, 14404, 14405, 14406, 14407, 14408, 14409, 14410, 14411, 14412, 14413, 14415, 14416, 14417, 14418, 14419, 14420, 14421, 14422, 14424, 14427, 14428, 14429, 14430, 14432, 14434, 14435, 14436, 14437, 14438, 14440, 14441, 14442, 14443, 14444, 14445, 14446, 14447, 14448, 14450, 14451, 14452, 14453, 14454, 14455, 14456, 14457, 14458, 14459, 14460, 14461, 14463, 14465, 14467, 14469, 14470, 14471, 14473, 14475 to distinguish chronic lymphocytic leukemia from normal B-cells.

19. The method of claim 1, wherein the signature comprises at least one sequence with identifiers selected from the group consisting of SEQ ID NOs: 24995-24996, 25025, 25031, 25033, 25087-25091, 25093-25094, 25128, 25150, 25161-25162, 25165, 25182, 25219-25220, 25230, 25277-25278, 25284, 25316, 25356-25357, 25359-25360, 25363-25364, 25397-25398, 25415, 25424, 25432, 25480, 25484-25486, 25498-25499, 25505, 25524, 25550-25552, 25570, 25580, 25583, 25609-25610, 25619, 25646-25647, 25685-25687, 25691, 25714, 25720, 25727-25728, 25731, 25741, 25746-25747, 25846-25847, 25868, 25882, 25904, 25908-25912, and 25914-25915 to distinguish B-cells from breast cells.

20. The method of claim 1, wherein the signature comprises at least one sequence with identifiers selected from the group consisting of SEQ ID NOs: 24880-24883, 24896-24897, 24959-24963, 24965, 24973, 25006, 25027, 25052, 25054, 25102-25103, 25110-25111, 25118, 25123, 25150, 25152-25153, 25183-25184, 25188, 25198, 25202, 25204-25206, 25210, 25212-25214, 25224-25225, 25245, 25252-25254, 25257, 25259-25261, 25270, 25273, 25286, 25294, 25296, 25313-25314, 25334, 25416, 25425, 25449-25450, 25454, 25476-25478, 25583, 25609-25612, 25665, 25667, 25705, 25714, 25786, 25894, and 25896-25897 to distinguish B-cells from white people from B-cells from black people.

21. The method of claim 1, wherein the signature comprises at least one sequence with identifiers selected from the group consisting of SEQ ID NO: 24881, 24926, 24952, 24981, 24990, 24995, 24998, 25010, 25047, 25051, 25075, 25101-25102, 2511 25111, 25118, 25121, 25149, 25211, 25218, 25238, 25309, 25359, 25373, 25376, 25386-25387, 25402, 25410, 25415-25416, 25420-25421, 25468, 25474, 25476, 25484-25487, 25493, 25524, 25536, 25560, 25596, 25604, 25620, 25631, 25651, 25662, 25664, 25714, 25723, 25803, 25829, 25850-25851, 25886-25887, 25898, 25902-25903, 25905, 25914, 25921, 25923, 25937 to distinguish B-cells from men from B-cells from women.

22. The method of claim 1, wherein the signature comprises at least one sequence with identifiers selected from the group consisting of SEQ ID NOs: 36100, 36101, 36105, 36107, 36111, 36112, 36114, 36115, 36116, 36119, 36120, 36121, 36122, 36123, 36139, 36143, 36146, 36147, 36148, 36149, 36155, 36156, 36157, 36163, 36171, 36173, 36176, 36177, 36178, 36179, 36180, 36181, 36182, 36183, 36188, 36189, 36194, 36197, 36200, 36203, 36204, 36215, 36217, 36218, 36219, 36222, 36223, 36227, 36228, 36230, 36231, 36234, 36238, 36239, 36240, 36241, 36242, 36243, 36246, 36248, 36252, 36254, 36262, 36265, 36266, 36269, 36270, 36271, 36272, 36273, 36276, 36278, 36279, 36282, 36285, 36287, 36288, 36289, 36293, 36294, 36295, 36296, 36297, 36298, 36299, 36303, 36304, 36305, 36306, 36307, 36308, 36313, 36319, 36320, 36322, 36323, 36326, 36327, 36331, 36332, 36333, 36335, 36336, 36338, 36339, 36341, 36342, 36344, 36347, 36355, 36356, 36357, 36372, 36373, 36374, 36375, 36376, 36378, 36381, 36384, 36387, 36391, 36392, 36395, 36397, 36399, 36400, 36401, 36405, 36406, 36408, 36409, 36428, 36429, 36430, 36431, 36432, 36433, 36435, 36436, 36437, 36444, 36450, 36451, 36452, 36453, 36455, 36456, 36457, 36460, 36461, 36462, 36463, 36464, 36465, 36466, 36467, 36468, 36469, 36470, 36471, 36472, 36478, 36485, 36490, 36491, 36498, 36499, 36504, 36505, 36506, 36507, 36508, 36509, 36510, 36511, 36512, 36513, 36517, 36520, 36521, 36523, 36524, 36529, 36530, 36533, 36534, 36535, 36538, 36539, 36541, 36542, 36543, 36544, 36545, 36546, 36547, 36550, 36553, 36554, 36561, 36562, 36572, 36573, 36574, 36575, 36578, 36579, 36580, 36581, 36582, 36584, 36586, 36589, 36590, 36591, 36593, 36594, 36597, 36599, 36600, 36601, 36607, 36608, 36609, 36610, 36611, 36612, 36614, 36615, 36616, 36617, 36618, 36619, 36620, 36621, 36627, 36628, 36629, 36637, 36638, 36639, 36640, 36641, 36642, 36643, 36644, 36645, 36646, 36647, 36649, 36650, 36658, 36665, 36669, 36670, 36671, 36673, 36674, 36675, 36676, 36677, 36678, 36679, 36680, 36682, 36683, 36684, 36689, 36690, 36691, 36692, 36693, 36694, 36695, 36696, 36697, 36698, 36701, 36702, 36703, 36705, 36706, 36707, 36708, 36709, 36710, 36711, 36712, 36714, 36715, 36716, 36718, 36719, 36720, 36721, 36722, 36726, 36727, 36728, 36729, 36730, 36731, 36732, 36733, 36734, 36735, 36738, 36739, 36741, 36742, 36744, 36745, 36746, 36747, 36749, 36751, 36754, 36755, 36756, 36757, 36759, 36760, 36761, 36762, 36763, 36764, 36765, 36768, 36769, 36770, 36771, 36772, 36775, 36776, 36777, 36778, 36788, 36789, 36793, 36794, 36796, 36797, 36798, 36799, 36800, 36803, 36805, 36806, 36809, 36810, 36812, 36814, 36817, 36825, 36826, 36827, 36829, 36830, 36831, 36832, 36834, 36835, 36838, 36839, 36841, 36844, 36846, 36848, 36849, 36851, 36854, 36855, 36857, 36859, 36860, 36861, 36862, 36863, 36864, 36868, 36869, 36871, 36872, 36877, 36878, 36879, 36880, 36881, 36883, 36884, 36885, 36886, 36887, 36889, 36890, 36891, 36892, 36895, 36897, 36901, 36902, 36903, 36904, 36905, 36907, 36909, 36910, 36911, 36913, 36914, 36915, 36916, 36917, 36918, 36919, 36925, 36931, 36938, 36939, 36941, 36942, 36945, 36946, 36948, 36952, 36953, 36955, 36956, 36957, 36958, 36961, 36963, 36964, 36965, 36967, 36968, 36973, 36976, 36977, 36978, 36979, 36980, 36981, 36982, 36983, 36985, 36988, 36989, 36990, 36991, 36992, 36997, 36998, 36999, 37001, 37004, 37005, 37008, 37009, 37012, 37013, 37014, 37021, 37022, 37023, 37024, 37025, 37026, 37029, 37032, 37033, 37036, 37039, 37044, 37046, 37048, 37049, 37050, 37051, 37054, 37055, 37056, 37057, 37058, 37059, 37060, 37063, 37065, 37066, 37075, 37077, 37078, 37079, 37080, 37081, 37083, 37087, 37088, 37089, 37090, 37091, 37094, 37095, 37099, 37100, 37101, 37110, 37115, 37116, 37117, 37119, 37120, 37121, 37123, 37124, 37125, 37127, 37132, 37133, 37134, 37135, 37137, 37138, 37139, 37141, 37142, 37143, 37144, 37145, 37146, 37149, 37150, 37151, 37152, 37155, 37157, 37160, 37161, 37162, 37163, 37164, 37165, 37166, 37167, 37168, 37169, 37171, 37174, 37175, 37177, 37178, 37181, 37182, 37183, 37184, 37185, 37187, 37193, 37194, 37195, 37196, 37197, 37198, 37199, 37201, 37202, 37203, 37206, 37207, 37208, 37209, 37211, 37213, 37214, 37216, 37217, 37226, 37227, 37228, 37229, 37230, 37231, 37234, 37235, 37237, 37244, 37245, 37247, 37248, 37249, 37251, 37253, 37254, 37255, 37261, 37262, 37265, 37271, 37272, 37273, 37274, 37278, 37279, 37283, 37303, 37304, 37305, 37306, 37307, 37308, 37312, 37316, 37319, 37321, 37323, 37324, 37325, 37326, 37327, 37334, 37335, 37336, 37337, 37338, 37339, 37340, 37341, 37342, 37348, 37356, 37363, 37365, 37368, 37369, 37370, 37372, 37374, 37375, 37376, 37382, 37383, 37385, 37386, 37388, 37391, 37394, 37395, 37398, 37400, 37401, 37402, 37403, 37404, 37405, 37407, 37408, 37410, 37419, 37420, 37422, 37423, 37424, 37425, 37426, 37429, 37430, 37431, 37432, 37433, 37445, 37446, 37448, 37449, 37453, 37454, 37456, 37461, 37462, 37463, 37464, and 37466 to distinguish normal pancreas from pancreatic cancer.

23. The method of claim 1, wherein the signature comprises at least one sequence with identifiers selected from the group consisting of SEQ ID NOs: 51377-51378, 51406, 51438, 51496, 51565, 51691, 51699, 51736-51737, 51745, and 51759 to distinguish platelets from people with a propensity to clot vs. platelets from people with a propensity to hemorrhage.

24. The method of claim 1, wherein the signature comprises at least one sequence with identifiers selected from the group consisting of SEQ ID NOs: 42434, 42520, 42537, 42577, 42751, 42979, 43019, 43090, 43128, 43156, 43310, 43352, 43398, 43426, 43437 to distinguish normal prostate from prostate cancer.

25. The method of claim 1, wherein characterizing the tRNA fragments comprises at least one assessment selected from the group consisting of sequencing the tRNA fragments, measuring overall abundance of one of the tRNA fragments mapped to the genome, measuring a relative abundance of the one tRNA fragment to a reference, assessing a length of the one tRNA fragment, identifying starting and ending points of the one tRNA fragment, identifying genomic origin of the one tRNA fragment, and identifying a terminal modification of the one tRNA fragment.

26. The method of claim 1, wherein the disease or condition, disease recurrence, or disease progression is selected from the group consisting of a cancer, and genetically predisposed disease or condition.

27. A method of diagnosing, identifying or monitoring breast cancer in a subject in need thereof, the method comprising:
isolating tRNA fragments from a cell obtained from the subject;
hybridizing the tRNA fragments to a panel of oligonucleotides engineered to detect tRNA fragments;
analyzing levels of the tRNA fragments present in the cell; wherein a differential in the measured tRNA fragments' levels to the reference is indicative of a diagnosis or identification of breast cancer in the subject; and
providing a treatment regimen to the subject dependent on the differential in measured tRNA fragments' levels to the reference.

28. A panel of engineered oligonucleotides comprising a mixture of oligonucleotides that are about 15 to about 40 nucleotides in length and capable of hybridizing tRNA fragments, wherein the tRNAs are less than 80 nucleotides in length.

29. A kit for high-throughput analysis of tRNAs fragments in a sample comprising:
the panel of engineered oligonucleotides of claim 28;
hybridization reagents; and
tRNA isolation reagents.

30. A method of identifying a cell's tissue of origin to treat a disease or condition, disease recurrence, or disease progression in a subject in need thereof comprising:
isolating fragments of tRNAs from a cell obtained from the subject;
characterizing the identity of the tRNA fragments and their relative abundance in the cell to identify a signature, wherein the signature is indicative of the cell's tissue of origin; and
providing a treatment regimen to the subject dependent on the cell's tissue of origin.

31. A method for identifying tRNA fragments comprising:
defining tRNA loci;
sequencing a population of RNA fragments;
mapping the sequenced RNA fragments to at least one tRNA genomic loci comprising:
disregarding mapped RNA fragments that differ in sequence from the tRNA genomic loci by at least an insertion, deletion, or replacement of a nucleotide;
adding back mapped RNA fragments that are post-transcriptionally modified that differ in sequence from the tRNA genomic loci only at the post-transcriptional modification;
excluding mapped RNA fragments that map to locations in the genome outside of the tRNA genomic loci; and
disregarding mapped RNA fragments with tRNA intron sequences; and
characterizing the mapped RNA fragments.

32. The method of claim 31, wherein the tRNA genomic loci comprise mitochondrial tRNA sequences from the mitochondrial genome, nuclear tRNA sequences from the nuclear genome, and mitochondria tRNA sequences from the nuclear genome.

33. The method of claim 31, wherein the mapped RNA fragments post-transcriptionally modified comprises at least one modified with a CCA trinucleotide at a 3′ end.

34. The method of claim 31, wherein characterizing the mapped RNA fragments comprises at least one assessment selected from the group consisting of identifying one or more of the mapped RNA fragments in a population, measuring an overall abundance of one or more of the mapped RNA fragments, measuring a relative abundance of one or more of the mapped RNA fragments to a reference, assessing a length of one or more of the mapped RNA fragments, identifying starting and ending points of one or more of the mapped RNA fragments, identifying genomic origin of one or more of the mapped RNA fragments, and identifying a terminal modification of one or more of the mapped RNA fragments.

35. A system for identifying tRNA fragments according to the method of claim 31 comprising a processor capable of analyzing the tRNA fragments.
